(19) United States
(12) Patent Application Publication
(10) Pub. No.: US 2022/0228120 A1
(43) Pub. Date: Jul. 21, 2022

(54) Cardiomyocyte Compositions and Use Thereof

(71) Applicants: UNIVERSITY HEALTH NETWORK, Toronto (CA); BlueRock Therapeutics LP, Cambridge, MA (US)

(72) Inventors: Gordon M. Keller, Toronto (CA); Shunsuke Funakoshi, Toronto (CA); lan Fernandes, Toronto (CA); Donghe Yang, Toronto (CA); Dan Charles Wilkinson, Jr., New York, NY (US)

(73) Assignees: UNIVERSITY HEALTH NETWORK, Toronto, ON (CA); BlueRock Therapeutics LP, Cambridge, MA (US)

(21) Appl. No.: 17/608,701

(22) PCT Filed: May 4, 2020
(86) PCT No.: PCT/US2020/031356

§ 371 Date: Nov. 3, 2021

Related U.S. Application Data
(60) Provisional application No. 62/843,118, filed on May 3, 2019.

(51) Int. Cl. C12N 5/077; A61K 35/34

(52) U.S. Cl. CPC C12N 5/0657; A61K 35/34; C12N 2506/45; C12N 2501/155; C12N 2501/415; C12N 2501/105

(57) ABSTRACT

Provided herein are enriched populations of ventricular compact cardiomyocytes and enriched populations of mature ventricular or atrial cardiomyocytes, as well as methods of generating the enriched cell populations and methods of using the enriched cell populations in regenerative cardiac cell therapies.

### CROSS REFERENCE TO RELATED APPLICATION

The present application claims priority from U.S. Provisional Application 62/843,118, filed May 3, 2019. The content of the aforementioned priority application is incorporated herein by reference in its entirety.

### BACKGROUND OF THE INVENTION

During fetal heart development, distinct subtypes of ventricular cardiomyocytes known as compact and trabecular, as well as atrial cardiomyocytes, are generated and contribute to different structures in the forming heart. Compact cardiomyocytes form the compact myocardium, the thick outer layer of the ventricular wall that provides the contractile force for pumping blood. Trabecular cardiomyocytes generate trabeculae myocardium that forms projections on the inner surface of the ventricle. Atrial cardiomyocytes refill ventricles with blood and are essential for fluid homeostasis. During development, factors secreted from the epicardium induce the specification and proliferation of the compact myocardium. At birth, the heart undergoes dramatic changes to mature into an organ capable of pumping blood throughout life. One of the most notable changes is in the production of energy within the cardiomyocytes that involves a switch from glycolysis to fatty acid oxidation (FAO). This switch is essential for the development of mature, functional cardiomyocytes.

Cardiomyocytes derived from human pluripotent stem cells can be used to establish new models to study cardiac diseases and to develop new cell therapies to treat the diseases. For these purposes, it is important to generate in tissue culture the cell type that corresponds to the target cells for a particular disease. In many cases, the desired cell type is mature ventricular cardiomyocytes of the compact lineage or subtype. Current differentiation protocols, however, often generate cell populations that contain a substantial portion of other cell types, such as cardiomyocytes of the trabecular lineage. Additionally, most of the current protocols promote the development of metabolically immature cells that depend on glycolysis as an energy source. Recent studies using cardiomyocytes derived from pluripotent stem cells for the treatment of cardiomyopathy in large animal models of myocardial infarction resulted in frequent ventricular tachyarrhythmias that were not observed prior to the cell transplantation. This presents a major preclinical hurdle to the successful use of in vitro derived cardiomyocytes in treating cardiac diseases. Thus, there remains a need for safe and efficacious cardiac regenerative therapy.

### SUMMARY OF THE INVENTION

The present disclosure provides a method of promoting differentiation of a ventricular cardiomyocyte progenitor cell into a ventricular compact cardiomyocyte, comprising contacting the progenitor cell with a Wnt signaling agonist and a cell proliferation stimulator, thereby obtaining a ventricular compact cardiomyocyte characterized by being Hairy/enhancer-of-split related with YRPW motif protein 2 (HEY2⁺), N-myc proto-oncogene protein (MYCN⁺), atrial natriuretic factor (ANF⁻) (e.g., cTNT⁺HEY2⁺MYCN⁺ANF⁻ or cTNT⁺HEY2^{high}MYCN⁺ANF⁻). In some embodiments, the cardiomyocyte progenitor cell is derived from a human pluripotent stem cell (PSC) such as an induced human PSC or a human embryonic stem cell. In some embodiments, the Wnt signaling agonist is an inhibitor of glycogen synthase kinase-3 β (GSK-3β) such as CHIR-99021, TWS119, BIO, SB 216763, SB 415286, and CHIR-98014. In particular embodiments, the inhibitor of GSK-3β is CHIR-99021. In some embodiments, the cell proliferation stimulator is insulin-like growth factor 2 (IGF2). In certain embodiments, the ventricular cardiomyocyte progenitor cell is contacted with CHIR-99021 at 0.1-10 μM and IGF2 at 1-50 ng/ml for 1-7 days. In further embodiments, the progenitor cell is contacted with CHIR-99021 at about 1 μM and IGF2 at about 25 ng/ml for about six days. The present disclosure also provides a plurality of ventricular compact cardiomyocytes obtainable by this method, as well as a pharmaceutical composition consisting of a cellular component and a carrier component, wherein the cellular component is a cell population in which more than 80% of the cells are ventricular compact cardiomyocytes characterized by being HEY2⁺ANF⁻BMP10⁻, and wherein the carrier component comprises a pharmaceutically acceptably carrier, optionally wherein the ventricular compact cardiomyocytes are characterized by being MYCN⁺.

In another aspect, the present disclosure provides a method of promoting metabolic maturation of a ventricular or atrial cardiomyocyte, comprising contacting an immature ventricular (e.g., ventricular compact) or atrial cardiomyocyte with a PPARα signaling agonist (e.g., GW7647, CP775146, fenofibrate, oleylethanolamide, palmitoylethanolamide, and WY14643), a hydrocortisone (e.g., dexamethasone), and a thyroid hormone (e.g., T3), thereby obtaining a mature ventricular (e.g., ventricular compact) or atrial cardiomyocyte, respectively. The method may further comprise culturing the immature ventricular or atrial cardiomyocyte in the presence of a fatty acid containing 16 or more carbons (e.g., palmitate or a derivative thereof), and/or in glucose. In particular embodiments, the method comprises culturing the immature ventricular or atrial cardiomyocyte in a culture medium containing GW7647, dexamethasone, T3, palmitate, and glucose for a period of about one to three weeks. In certain embodiments, the method comprising culturing the immature ventricular or atrial cardiomyocyte in a culture medium containing about 1 μM GW7647, about 100 ng/ml dexamethasone, about 4 nM T3, about 200 μM palmitate, and about 2 mg/ml glucose for a period of about one to two weeks (e.g., 5, 6, 7, 8, 9, 10, 11, 12, 13, or 14 days), wherein the culture medium is optionally agitated during the culturing step.

In another aspect, the present disclosure provides a method of generating a cell population enriched for mature ventricular compact cardiomyocytes, comprising contacting a population of a ventricular cardiomyocyte progenitor cell with a Wnt signaling agonist and a cell proliferation stimulator, thereby obtaining a first cell population comprising immature ventricular compact cardiomyocytes, contacting the first cell population with a Wnt signaling antagonist (Xav-939), and culturing the contacted first cell population in the presence of a PPARα signaling agonist, a hydrocortisone, and a thyroid hormone, thereby obtaining a second cell population enriched for mature ventricular compact cardiomyocytes, as described above. In particular embodiments, the method comprises contacting the population of the ventricular cardiomyocyte progenitor cells with CHIR-99021 at about 1 μM and IGF2 at about 25 ng/ml for about six days to obtain the first cell population, contacting the first cell population with about 4 μM Xav-939 for about two days, and culturing the contacted first cell population in a culture medium containing about 1 μM GW7647, about 100 ng/ml dexamethasone, about 4 nM thyroid hormone T3, about 200 μM palmitate, and about 2 g/L glucose for a period of about two weeks, wherein the cell culture is agitated during the culturing step. In some embodiments, the method comprises isolating the mature cardiomyocytes from the cell culture (e.g., through fluorescence- or magnetic-activated cell sorting) by using binding agents (e.g., antibodies or antigen-binding fragments thereof) that bind cell surface markers LDLR and CD36.

In some embodiments, an immature ventricular cardiomyocyte may be characterized by being HEY2⁺MYCN⁺ANF⁻. In some embodiments, a mature ventricular compact cardiomyocyte is characterized by being MLC2V⁺HEY2⁺ANF⁻BMP10⁻ and/or further characterized by one or both of the following features: i) expressing (e.g., at a high level) one or more markers selected from CD36, LDLR, FABP3, ACSL1, COX6A2, ATP5A1, COX7A1, CKMT2, SOD2, ASB2, FGF12, CPT1B, MLYCD, PDK4, TCAP, KCNJ2, ATP2A2, ADRB1, UCP2, UCP3, TP53INP2, NMRK2, NPPB, HSPB6, KLF9, CEBPB, MASP1, HRC, ACSL1, ESRRA, and SCD (e.g., one or more markers selected from CD36, LDLR, NMRK2, NPPB, HSPB6, KLF9, CEBPB, and ESRRA, such as CD36/LDLR/NMRK2, CD36/NMRK2, or CD36/LDLR); and ii) increased (a) mitochondria mass, (b) sarcomere length, (c) conduction velocity, and/or (d) contractile force, compared to an immature ventricular compact cardiomyocyte.

In some embodiments, an immature atrial cardiomyocyte is characterized by being cTNT⁺MLC2V⁻. In some embodiments, a mature atrial cardiomyocyte is characterized by being KCNA5⁺KCNJ3⁺GJA5⁺NR2F2⁺MLC2V⁻ and/or further characterized by one or both of the following features: i) expressing (e.g., at a high level) one or more of markers selected from FABP3, MLYCD, ATP5A1, COX7A1, CKMT2, KCNJ2, TCAP, and CD36; and ii) increased (a) mitochondria mass, (b) sarcomere length, and/or (c) maximal respiration, compared to immature atrial cardiomyocytes.

The present disclosure also provides a plurality of mature cardiomyocytes obtained by these methods. Also provided are pharmaceutical compositions consisting of a cellular component and a carrier component, wherein the cellular component is a cell population in which more than 80% of the cells are mature ventricular compact cardiomyocytes or mature atrial cardiomyocytes, and wherein the carrier component comprises a pharmaceutically acceptable carrier. Further provided are an aggregate of cells (e.g., three-dimensional organoids) in cell culture comprising the cell populations provided herein.

In yet another aspect, the present disclosure provides a method of treating a cardiomyopathy condition, comprising administering to a subject (e.g., a human patient) in need thereof the cells or pharmaceutical compositions provided herein. The cardiomyopathy may be, for example, myocardial infarction or heart failure, optionally wherein the heart failure is left-sided heart failure, a right-sided heart failure, a diastolic heart failure, a systolic heart failure, or congestive heart failure. Also provided are cells and pharmaceutical compositions for use in treating a cardiomyopathy condition, and the use of the present cells in the manufacture of a medicament for treating a cardiomyopathy condition.

In a further aspect, the present disclosure provides a method of detecting the presence of a mature ventricular compact cardiomyocyte in a cell population, comprising detecting a cell that expresses one or more markers selected from CD36, LDLR, FABP3, ACSL1, COX6A2, ATP5A1, COX7A1, CKMT2, SOD2, ASB2, FGF12, CPT1B, MLYCD, PDK4, TCAP, KCNJ2, ATP2A2, ADRB1, UCP2, UCP3, TP53INP2, NMRK2, NPPB, HSPB6, KLF9, CEBPB, MASP1, HRC, ACSL1, ESRRA, and SCD (e.g., one or more markers selected from CD36, LDLR, NMRK2, NPPB, HSPB6, KLF9, CEBPB, and ESRRA, such as CD36/LDLR/NMRK2, CD36/NMRK2, or CD36/LDLR), wherein the detected cell is a mature ventricular compact cardiomyocyte.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIGS. 1A-H are a panel of graphs and photographs showing the generation of compact cardiomyocytes.

FIG. 1A is a t-Distributed Stochastic Neighbor Embedding (t-SNE) plot of day 20 ventricular cardiomyocytes in tissue culture, showing 9 different cell clusters.

FIG. 1B is a t-SNE plot displaying HEY2⁺ and ANF⁺ populations.

FIG. 1C is a diagram showing the signaling pathways upregulated in the HEY2^{high} cells as compared to the ANF^{high} cells.

FIG. 1D is a diagram illustrating a protocol for generating compact cardiomyocytes in vitro. CHIR: CHIR-99021.

FIG. 1E is a graph comparing the relative number of cardiomyocytes in the day 16 cell populations treated with CHIR, IGF2, CHIR+IGF2, or Xav-939 (XAV) as compared to the number of cardiomyocytes in the cell population treated with DMSO (negative control). *p<0.05, **p<0.01, ***p<0.001 by one-way ANOVA with Tukey's multiple comparisons. CM: cardiomyocytes. All error bars represent standard error of the mean (SEM). CHIR: 1 μM. IGF2: 25 ng/ml. XAV: 4 μM.

FIG. 1F is a panel of four graphs showing RT-qPCR expression analyses of compact (HEY2 and MYCN) and trabecular (ANF and BMP10) markers in the indicated cell populations. *p<0.05, **p<0.01, ***p<0.001 by one-way ANOVA with Tukey's multiple comparisons. All error bars represent SEM. NRG: neuregulin 1. Fetal LV: human fetal left ventricular tissue. Fetal RA: human fetal right atrial tissue.

FIG. 1G is a panel of three photographs showing representative immunostaining of compact (HEY2 and MYCN; CHIR+IGF2 treated), trabecular (ANF and BMP10; NRG treated), and control (negative; DMSO treated) cardiomyocytes. Red fluorescence: HEY2. Green fluorescence: ANF. Blue fluorescence: DAPI. Scale bar: 100 μm.

FIG. 1H is a graph quantifying the percentage of HEY2^{low}ANF⁺ cardiomyocytes in compact, trabecular, and non-treated (control) ventricular cardiomyocytes. **p<0.01, ***p<0.001 by one-way ANOVA with Tukey's multiple comparisons. All error bars represent standard error of the mean (SEM).

FIGS. 2A-K are a panel of graphs and photographs showing that PPARα agonist, dexamethasone, thyroid hormone (T3), and palmitate induced a FAO program in compact cardiomyocytes.

FIG. 2A is a graph showing the proportion of cardiac troponin T (cTNT) positive cardiomyocytes were detected at day 18 in compact ventricular populations cultured for 2 days in the presence or absence of Xav-939 (XAV). “Ventricular”: control cell population that was not specified to a compact fate with CHIR and IGF2. **p<0.01, ***p<0.001 by one-way ANOVA with Tukey's multiple comparisons. All error bars represent SEM.

FIG. 2B is a schematic overview of energy metabolism in cardiomyocytes. FFA: free fatty acids. FAO: fatty acid oxidation.

FIG. 2C is a panel of graphs showing representative flow cytometric analyses of CD36 and SIRPA expression in the day 18 population and the derivative day 32 populations cultured in (i) DMSO-control (negative), (ii) GW7647 (PPARα agonist), (iii) dexamethasone (Dex) and thyroid hormone (T3), or (iv) the combination of GW7647, Dex, and T3.

FIG. 2D is a panel of cell graphs showing representative flow cytometric analyses of CD36 and SIRPA expression in the day 18 population and the derivative day 32 populations cultured in either DMSO-control (highG), GW7647 (PPARα agonist), GW7647 and palmitate (Pal) (200 μM), the combination of GW7647, palmitate, and dexamethasone (Dex), the combination of GW7647, palmitate, and thyroid hormone (T3) or the combination of GW7647, palmitate, Dex, and T3.

FIG. 2E is a graph quantifying the CD36⁺SIRPA⁺ populations in day 32 populations cultured in the indicated conditions from day 18 to day 32. ***p<0.001 by one-way ANOVA with Tukey's multiple comparisons. All error bars represent SEM. Nega: negative control. GW: GW7647.

FIG. 2F is a panel of graphs showing representative flow cytometric analyses of CD36 and SIRPA expression in day 32 populations treated with GW7647+Dex+T3 in media containing high glucose (4.5 g/L), low glucose (2 g/L) or no glucose (0 g/L) in the presence or absence of palmitate (200 μM). Cells were cultured in 24-well culture dishes.

FIG. 2G is a panel of two photomicrographs showing cardiomyocyte aggregates in a 24-well culture dish without rotation (upper image) or in a 10-cm dish with rotation (70 rpm) (lower image).

FIG. 2H is a panel of graphs showing representative flow cytometric analyses of CD36 and SIRPA expression on day 32 cells treated with GW+Dex+T3 in media containing high glucose (4.5 g/L), low glucose (2 g/L) or no glucose (0 g/L) in the presence or absence of palmitate (200 μM). Cells were cultured in a 10-cm dish with constant rotation.

FIG. 2I is a graph quantifying by flow cytometry the mean fluorescent intensity (MFI) of CD36 expression on cells cultured under the indicated conditions. *p<0.05, **p<0.01, ***p<0.001 by one-way ANOVA with Tukey's multiple comparisons. All error bars represent SEM.

FIG. 2J is a panel of graphs showing RT-qPCR expression analyses of FAO-related genes in day 32 cells cultured in high glucose media without factors (control) and with GW+Dex+T3 in low glucose media with palmitate (day 32 mature). *p<0.05, **p<0.01 by unpaired t-test. All error bars represent SEM.

FIG. 2K is a panel of graphs showing RT-qPCR expression analyses of FAO-related genes in day 32 cells cultured in high glucose media without factors (control), low glucose media with palmitate, high glucose media with Dex and T3, or in the presence of GW+palmitate+Dex+T3 in low glucose media. *p<0.05, ***p<0.001 by one-way ANOVA with Tukey's multiple comparisons. All error bars represent SEM. LV: left ventricular tissue.

FIGS. 3A-E are a panel of graphs and photographs showing the metabolic profiles of mature compact cardiomyocytes.

FIG. 3A is a panel of representative kinetic graphs of oxygen consumption rate (OCR) as assayed by FAO Cell Mito stress test assay in day 16 immature cardiomyocytes (day 16 immature), day 32 cardiomyocytes cultured in high glucose media (day 32 highGlucose), and day 32 mature cardiomyocytes treated with GW+Dex+T3 in low glucose media supplemented by palmitate (day 32 mature). Blue line: cells treated with palmitate. Green line: cells treated with bovine serum albumin (BSA) (control). Red line: cells treated with etomoxir (ETO, 40 μM)+palmitate.

FIG. 3B is a panel of graphs showing the comparison of each parameter in FAO Cell Mito stress assay in day 16 immature, day 32 high Glucose, and day 32 mature cardiomyocytes. All error bars represent SEM.

FIG. 3C is a panel of graphs showing RT-qPCR expression analyses of UCP2 and UCP3 (top two panels) in day 32 high Glucose cardiomyocytes and day 32 mature cardiomyocytes, and RT-qPCR expression analyses of SOD2 and UCP2 (bottom panel) in the cells cultured under the indicated conditions. All error bars represent SEM. Control: CMs cultured in high glucose media. Pal: CMs cultured with Palmitate in low glucose media. DT: CMs cultured with the combination of Dex and T3 in high glucose media. PPDT: CMs cultured with PPARA agonist, Palmitate, Dex and T3 in low glucose media. In the top two panels, ***p<0.001 by unpaired t-test. In the bottom panel, *p<0.05, **p<0.01, ***p<0.001 by one-way ANOVA with Tukey's multiple comparisons.

FIG. 3D is a panel of photographs showing representative transmission electron microscope image of lipid droplets in day 32 high Glucose cardiomyocytes and day 32 mature cardiomyocytes. Black round shaped lipid droplets are more frequently observed in day 32 mature cardiomyocytes.

FIG. 3E is a graph quantifying by Nile Red staining lipid storage in day 32 high Glucose cardiomyocytes and day 32 mature cardiomyocytes. ***p<0.001 by unpaired t-test. All error bars represent SEM.

FIGS. 4A-D are a panel of graphs showing that transient activation of the FAO pathway improves metabolic profiles in mature compact cardiomyocytes.

FIG. 4A is a schematic diagram comparing oxygen consumption rates (OCR) in the following four conditions: 1) cardiomyocytes treated with GW+Dex+T3 in low glucose media with palmitate from days 18 to 32; 2) cardiomyocytes treated with GW+Dex+T3 in low glucose media with palmitate from days 18 to 27, followed by treatment of GW in low glucose media with palmitate from days 28 to 32; 3) cardiomyocytes treated with GW+Dex+T3 in low glucose media with palmitate from days 18 to 27, followed by culture in low glucose media with palmitate from days 28 to 32; and 4) cardiomyocytes treated with GW+Dex+T3 in low glucose media with palmitate from days 18 to 27, followed by culture in low glucose media from days 28 to 32. OCR measurement was performed at day 32.

FIG. 4B is a panel of four representative kinetic graphs of OCRs measured for the four conditions in FIG. 4A. Blue line: palmitate. Green line: BSA control. Red line: etomoxir (ETO, 40 μM)+palmitate.

FIG. 4C is a panel of graphs comparing each parameter in FAO Cell Mito stress assay in day 32 cardiomyocytes cultured in high glucose media, and day 32 cardiomyocytes cultured in the four conditions in FIG. 4A. All error bars represent SEM.

FIG. 4D is a diagram illustrating a protocol for generating mature ventricular compact cardiomyocytes.

FIGS. 5A-F are a panel of graphs and photographs showing single cell RNA sequencing analysis of mature compact CMs.

FIG. 5A shows UMAP (Uniform Manifold Approximation and Projection) projections of day 32 immature and mature cardiomyocytes (revealing 5 different clusters) and UMAP plot displaying TNNT2 and representative genes in cluster 2, 3, and 4.

FIG. 5B shows a UMAP projection revealing 5 different cell populations and UMAP plot displaying representative FAO, mitochondrial genes.

FIG. 5C is a panel of violin plots of the significantly differentially enriched Gene Ontology (GO) terms in mature CM cluster (cluster 0, red) than in immature CM cluster (cluster 1, green).

FIG. 5D is a panel of violin plots showing Gene Ontology (GO) terms that are more significantly enriched expressed in clusters A and B than in other clusters.

FIG. 5E shows a UMAP plot displaying highly expressed genes in cluster A, including CD36, LDLR, ASB2, and FGF12.

FIG. 5F is a representative flow cytometric analyses of CD36 and LDLR expression in the day 18 population and the derivative day 25 and day 32 populations cultured in high glucose media (immature CM) and PPDT/Pal protocol (mature CM).

FIGS. 6A-Q are a panel of graphs and photographs showing the characteristics of metabolically mature compact cardiomyocytes. All error bars represent SEM. Control: CMs cultured in high glucose media. Pal: CMs cultured with Palmitate in low glucose media. DT: CMs cultured with the combination of Dex and T3 in high glucose media. PPDT/PAL (mature): CMs cultured with PPARA agonist, Palmitate, Dex and T3 in low glucose media followed by Palmitate in low glucose media.

FIG. 6A is a panel of graphs showing RT-qPCR expression analyses of sarcomere genes (MYL2, TCAP), ion channel genes (KCNJ2, HCN4), calcium handling gene (ATP2A2), mitochondrial genes (COX3, COX7A1), and ADRB1 in day 32 populations cultured in high glucose media without factors (day 32 high Glucose) and day 32 mature cardiomyocytes (day 32 mature). *p<0.05, **p<0.01 by unpaired t-test. All error bars represent SEM.

FIG. 6B is a panel of photographs showing representative cTNT immunostaining of the cells under the indicated conditions. Green fluorescent: cTNT. Blue fluorescent: DAPI. Scale bar: 100 μm.

FIGS. 6C and 6D compare cell size (FIG. 6C) and percentage of binucleated CMs in the cells (FIG. 6D) under the indicated conditions. *p<0.05, **p<0.01, ***p<0.001 by one-way ANOVA with Tukey's multiple comparisons. All error bars represent SEM.

FIG. 6E is a panel of photographs showing representative mitochondria imaging in transmission electron microscope (TEM) in day 32 high Glucose cardiomyocytes and day 32 mature cardiomyocytes.

FIG. 6F is a graph showing the comparison of mitochondria size in day 32 highGlucose cardiomyocytes and day 32 mature cardiomyocytes. ***p<0.001 by unpaired t-test.

FIG. 6G is a graph showing the comparison of mitotracker fluorescent intensity between day 32 high Glucose cardiomyocytes and day 32 mature cardiomyocytes. *p<0.05 by unpaired by flow cytometry t-test. All error bars represent SEM.

FIG. 6H compares mitochondria size based on TEM in the cells under the indicated conditions. ***p<0.001 by one-way ANOVA with Tukey's multiple comparisons. All error bars represent SEM.

FIG. 6I is a panel of photographs showing representative images of sarcomere structure in day 32 high Glucose cardiomyocytes and day 32 mature cardiomyocytes (TEM). Left: low magnification. Right: high magnification. Scale bar: 1 μm. ***p<0.001 by one-way ANOVA with Tukey's multiple comparisons. All error bars represent SEM.

FIG. 6J is a graph comparing sarcomere lengths (based on TEM analyses) in day 32 high Glucose cardiomyocytes and day 32 mature cardiomyocytes. ***p<0.001 by unpaired t-test.

FIG. 6K compares sarcomere length (based on TEM analyses) in the cells under the indicated conditions. ***p<0.001 by one-way ANOVA with Tukey's multiple comparisons. ***p<0.001 by one-way ANOVA with Tukey's multiple comparisons. All error bars represent SEM.

FIG. 6L is a panel of images showing representative optical mapping measurements of conduction velocity (CV) in day 32 high Glucose cardiomyocytes and day 32 mature cardiomyocytes.

FIG. 6M is a graph comparing the CV values between day 32 high Glucose cardiomyocytes and day 32 mature cardiomyocytes. All error bars represent SEM.

FIGS. 6N and 6O represent Ca²⁺ transient analyses of the cells under the indicated conditions. FIG. 6N is a representative Ca²⁺ transient in day 32 CMs cultured in high glucose media (control) and mature CMs (mature). FIG. 6O is a comparison of the parameters in Ca²⁺ transient in the cells under the indicated conditions. **p<0.01, ***p<0.001 by one-way ANOVA with Tukey's multiple comparisons. ns: not significant. All error bars represent SEM.

FIG. 6P shows a comparison of the percentage of Ki67(+) CMs among cTNT(+) cells in the cells under the indicated conditions. *p<0.05 by student t-test. ns: not significant. All error bars represent SEM.

FIG. 6Q shows a comparison of the contraction force measured by biowire cardiac tissues under the indicated conditions. *p<0.05, **p<0.01 by one-way ANOVA with Tukey's multiple comparisons. All error bars represent SEM.

FIGS. 7A-K illustrate the induction of metabolic maturation in hPSC-derived atrial CMs. All error bars represent SEM. Fetal RA: fetal right atrial tissue. Fetal LV: fetal left ventricular tissue.

FIG. 7A is a representative transmission electron microscope (TEM) image showing sarcomere structure in the atrial CMs cultured in high glucose media (control) and mature atrial CMs. Scale bar: 1 μm.

FIG. 7B shows a comparison of sarcomere length (based on TEM analyses) in the cells under the indicated conditions. **p<0.01, ***p<0.001 by one-way ANOVA with Tukey's multiple comparisons. ns: not significant. All error bars represent SEM.

FIG. 7C shows a representative TEM image of mitochondria in the cells under the indicated conditions. Scale bar: 1 μm.

FIG. 7D shows a comparison of mitochondria size based on TEM in the cells under the indicated conditions. **p<0.01, ***p<0.001 by one-way ANOVA with Tukey's multiple comparisons. All error bars represent SEM.

FIG. 7E shows a representative flow cytometric analyses of CD36 and SIRPA expression in the atrial CMs cultured in high glucose media (control) and mature atrial CMs.

FIG. 7F shows the quantification of CD36⁺/SIRPA⁺ populations in the cells under the indicated conditions. ***p<0.001 by one-way ANOVA with Tukey's multiple comparisons. All error bars represent SEM.

FIG. 7G shows an RT-qPCR expression analyses of FAO-related genes in the cells cultured under the indicated conditions. *p<0.05, **p<0.01, ***p<0.001 by one-way ANOVA with Tukey's multiple comparisons. All error bars represent SEM.

FIG. 7H shows an RT-qPCR expression analyses of mitochondrial genes in the cells under the indicated conditions. RT-qPCR expression analyses of mitochondrial genes in the cells under the indicated conditions. *p<0.05, ***p<0.001 by one-way ANOVA with Tukey's multiple comparisons. All error bars represent SEM.

FIG. 7I shows an RT-qPCR expression analyses of TCAP, KCNJ2, and HCN4 in the cells under the indicated conditions. Fetal RA: Fetal right atrial tissue, Fetal LV: Fetal left ventricular tissue. *p<0.05, **p<0.01, *** p<0.001 by student-t test. All error bars represent SEM. ns: not significant.

FIG. 7J shows a representative kinetic graph of OCR by FAO Cell Mito stress test assay in control-atrial CMs and mature atrial CMs. Blue: palmitate. Green: BSA control. Red: etomoxir (ETO, 40 μM)+palmitate.

FIG. 7K shows a comparison of each parameter in FAO Cell Mito stress assay in the cells cultured under the indicated conditions. *p<0.05, **p<0.01, ***p<0.001 by one-way ANOVA with Tukey's multiple comparisons. All error bars represent SEM.

FIGS. 8A-O illustrate an in vitro model of pathological adaptation and in vivo transplantation of mature cells into infarcted rat hearts.

FIG. 8A shows an RT-qPCR expression analyses of ADRB1 in immature and mature cells. *p<0.05 by student-t test. All error bars represent SEM.

FIG. 8B shows a protocol for the model under the pathological stimuli in vitro.

FIG. 8C shows an RT-qPCR expression analyses of the glycolysis-related genes and TAG synthesis-related genes in immature and mature cells with and without the pathological stimuli (hypoxia+ISO). *p<0.05, **p<0.01, ***p<0.001 by student-t test. All error bars represent SEM. ns: not significant.

FIG. 8D shows (left) a representative kinetic graph of ECAR by Seahorse™ XF assay in immature and mature cells with and without the pathological stimuli (hypoxia+ISO), and (right) a comparison of glycolysis based on ECAR measurement by Seahorse™ XF assay. *p<0.05, **p<0.01 by student-t test. All error bars represent SEM.

FIG. 8E shows the quantification of lipid storage in the cells by Nile Red staining in immature and mature cells with and without the pathological stimuli (hypoxia+ISO). **p<0.01 by student-t test.

FIG. 8F shows an RT-qPCR expression analyses of PLIN2 and HSL in immature and mature cells with and without the pathological stimuli (hypoxia+ISO). *p<0.05 by student-t test. All error bars represent SEM. ns: not significant.

FIG. 8G shows an RT-qPCR expression analyses of CASP9 in immature and mature cells with and without the pathological stimuli (hypoxia+ISO). *p<0.05, **p<0.01 by student-t test. All error bars represent SEM. ns: not significant.

FIG. 8H shows a comparison of Annexin V(+) cells based on flow cytometric analyses. **p<0.01 by student-t test. All error bars represent SEM. ns: not significant.

FIG. 8I shows a representative flow cytometric analyses of Annexin V in immature and mature cells with and without the pathological stimuli (hypoxia+ISO).

FIG. 8J shows a summary of the activated pathways in mature cells with the pathological stimuli (hypoxia+ISO). Black arrows show the activated (or downregulated) genes and parameters in mature cells with pathological stimuli.

FIG. 8K shows a comparison of graft size between immature cell transplantation and mature cell transplantation. All error bars represent SEM.

FIG. 8L shows a comparison of sarcomere length between the grafted immature cells and the grafted mature cells. **p<0.01 by student-t test. All error bars represent SEM.

FIG. 8M shows a comparison of the percentage of Ki67(+) CMs between the grafted immature cells and the grafted mature cells. ***p<0.001 by student-t test. All error bars represent SEM.

FIG. 8N shows an RT-qPCR expression analyses of CX43 in immature and mature cells. *p<0.05, ***p<0.001 by student-t test. All error bars represent SEM.

FIG. 8O shows a comparison of CX43 expression between the grafted immature cells and the grafted mature cells. ***p<0.001 by student-t test. All error bars represent SEM.

### DETAILED DESCRIPTION OF THE INVENTION

An important goal in cardiovascular regenerative medicine is to develop cell-based therapies to remuscularize the left ventricle wall of a patient's heart following a myocardial infarction (a heart attack). The cells most often damaged in myocardial infarct are mature ventricular compact cardiomyocytes, making these cells an ideal target population for remuscularization of the damaged ventricle. The present invention provides methods of generating mature ventricular cardiomyocytes (e.g., mature ventricular compact cardiomyocytes) or mature atrial cardiomyocytes from human pluripotent stem cells (hPSCs) or immature cardiomyocytes by manipulating key regulatory pathways that promote cardiomyocyte specification (e.g., compact specification), proliferation, and metabolic maturation.

The present disclosure provides methods that generate cardiomyocyte cell populations enriched for ventricular compact cardiomyocytes (e.g., more than 80% (such as more than 85%, more than 90%, more than 95%, or more than 99%) of the ventricular cardiomyocytes are ventricular compact cardiomyocytes).

The present disclosure also provides methods that generate ventricular cardiomyocyte cell populations enriched for mature ventricular compact cardiomyocytes (e.g., more than 50% (such as more than 55%, more than 60%, more than 65%, more than 75%, more than 80%, more than 90%, more than 95%, or more than 99%) of the cells in the ventricular cardiomyocyte cell population are mature ventricular compact cardiomyocytes).

The present disclosure further provides methods that generate atrial cardiomyocyte cell populations enriched for mature atrial cardiomyocytes (e.g., more than 50% (such as more than 55%, more than 60%, more than 65%, more than 75%, more than 80%, more than 90%, more than 95%, or more than 99%) of the cells in the atrial cardiomyocyte cell population are mature atrial compact cardiomyocytes).

The enriched populations of mature ventricular compact cardiomyocytes provided herein are expected to be more efficacious in regenerative medicine for repairing damaged or underdeveloped ventricle walls. These cells will engraft an infarcted heart more efficiently, integrate with the host myocardium more rapidly, improve ventricular wall thickening, and/or improve ejection fraction as compared to pharmaceutical compositions containing higher percentages of immature cells or other cell types. The present mature cells also will replicate specific disease states in vitro better than immature cells. Compared to current therapies (including those in clinical and pre-clinical development), cell therapies using the present enriched cell populations will induce fewer ventricular arrhythmias.

Access to enriched ventricular or atrial cardiomyocyte populations is of value for in vitro studies of human cardiac physiology, as well as for patient-specific disease modelling. These disease modelling studies will enable identification of potential drug targets and development of novel drug treatments. In addition, hPSC-derived cardiomyocytes could also be used to assess potentially dangerous off-target effects of novel drugs on heart tissue in safety pharmacology screens.

### Generation of Ventricular and Atrial Cardiomyocytes in vitro

Cardiomyocytes are cells characterized by the expression of one or more of cardiac troponins (e.g., cardiac troponin I or cardiac troponin T (“cTNT”)). Ventricular mesodermal cells (e.g., those seen in the ventricular mesoderm) are cells that are on a developmental path to become ventricular cardiomyocytes (as opposed to becoming other types of cardiomyocytes such as atrial or pacemaker cells). Ventricular mesodermal cells are characterized by being CD235a⁺ALDH⁻ (ALDH: aldehyde dehydrogenase). Ventricular cardiomyocyte progenitor cells are cells that are further along the developmental path to become ventricular cardiomyocytes, relative to ventricular mesodermal cells. Ventricular cardiomyocyte progenitor cells are characterized by being cTNT⁺NKX2-5⁺. Ventricular cardiomyocytes are cardiomyocytes having ventricular properties, including expression of ventricular-specific markers such as myosin light chain 2v (MLC2V), myosin light chain 2 (MYL2), Iroquois homeobox protein 4 (IRX4), and NK2 homeobox 5 (NKX2-5), and/or displaying electrophysical properties of a ventricular cell. In some embodiments, ventricular cardiomyocytes are characterized by being cTNT⁺MLC2V⁺. Ventricular cardiomyocytes include cells of the compact lineage and cells of the trabecular lineage.

Atrial mesodermal cells (e.g., those seen in the atrial mesoderm) are cells that are on a developmental path to become atrial cardiomyocytes. Atrial mesodermal cells are characterized by being RALDH2⁺CD235a⁻ (RALDH2: retinaldehyde dehydrogenase 2). Atrial cardiomyocyte progenitor cells are cells that are further along the developmental path to become atrial cardiomyocytes, relative to atrial mesodermal cells. Immature atrial cardiomyocytes are characterized by being cTNT⁺MLC2V⁻. Atrial cardiomyocytes are cardiomyocytes having atrial properties, including expression of atrial-specific markers such as potassium voltage-gated channel subfamily A member 5 (KCNA5), KCNJ3, gap junction protein alpha 5 (GJA5) (aka CX40), and nuclear receptor subfamily 2 group F member 2 (NR2F2).

A variety of cell types may be used as a source of cells for the in vitro (including ex vivo) generation of ventricular or atrial cardiomyocytes. The source cells may be, for example, pluripotent stem cells (PSCs). In other embodiments, the source cells may be mesodermal cells. As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, embryonic stem cells, PSCs derived by somatic cell nuclear transfer, and induced PSCs (iPSCs). As used herein, the term “embryonic stem cells,” “ES cells,” or “ESCs” refers to pluripotent stem cells obtained from early embryos; in some embodiments, this term refers to ES cells obtained from a previously established ES cell line and excludes stem cells obtained by destruction of a human embryo.

One convenient source of cells for generating ventricular and atrial cardiomyocytes is iPSCs. iPSCs are a type of pluripotent stem cell artificially generated from a non-pluripotent cell, such as an adult somatic cell or a partially differentiated cell or terminally differentiated cell (e.g., a fibroblast, a cell of hematopoietic lineage, a myocyte, a neuron, an epidermal cell, or the like), by introducing to the cell or contacting the cell with one or more reprogramming factors. Methods of producing iPSCs are known in the art and include, for example, inducing expression of one or more genes (e.g., POU5F1/OCT4 (Gene ID: 5460) in combination with, but not restricted to, SOX2 (Gene ID: 6657), KLF4 (Gene ID: 9314), c-MYC (Gene ID: 4609, NANOG (Gene ID: 79923), and/or LIN28/LIN28A (Gene ID: 79727)). Reprogramming factors may be delivered by various means (e.g., viral, non-viral, RNA, DNA, or protein delivery); alternatively, endogenous genes may be activated by using, e.g., CRISPR and other gene editing tools, to reprogram non-pluripotent cells into PSCs.

Methods of isolating and maintaining PSCs, including ESCs and iPSCs, are well known in the art. See, e.g., Thomson et al., Science (1998) 282(5391):1145-7; Hovatta et al., Human Reprod. (2003) 18(7):1404-09; Ludwig et al., Nat Methods (2006) 3:637-46; Kennedy et al., Blood (2007) 109:2679-87; Chen et al., Nat Methods (2011) 8:424-9; and Wang et al., Stem Cell Res. (2013) 11(3):1103-16.

Methods for inducing differentiation of PSCs into cells of various lineages are well known in the art. For example, numerous methods exist for differentiating PSCs into cardiomyocytes, as shown in, e.g., Kattman et al., Cell Stem Cell (2011) 8(2):228-40; Burridge et al., Nat Protocols (2014) 11(8):855-60; Burridge et al., PLoS ONE (2011) 6:e18293; Lian et al., PNAS. (2012) 109:e1848-57; WO 2016/131137; WO 2018/098597; and U.S. Pat. No. 9,453,201. See also Lee et al., Cell Stem Cell (2017) 21:179-94, which describes methods for differentiating human ESCs and human iPSCs into ventricular and atrial cardiomyocytes.

Multipotent cells such as human mesodermal cells and cardiac progenitor cells may also be used. As used herein, a “multipotent” cell refers to a cell that is capable of giving rise to more than one cell type upon differentiation. Multipotent cells have more limited differentiation potential than pluripotent cells.

In some other embodiments, the source of cells is differentiated somatic cells that may be reprogrammed into cardiomyocyte cells. For example, the source of cells may be fibroblasts (see, e.g., Engel and Ardehali, Stem Cells Int. (2018) 2018:1-10). Direct reprogramming of fibroblasts into cardiomyocyte-like cells by overexpressing the cardiac developmental transcription factors Gata4, Mef2c, and Tbx5 (GMT) has been reported (Ieda et al., Cell. (2010) 142(3):375-86).

Developmentally, cardiomyocyte progenitor cells or cardiac progenitor cells are derived from cardiac mesodermal cells, and are characterized by being cTNT⁺. One method for generating human cardiac progenitors from hPSCs (e.g., hESCs and human iPSCs) involves (i) inducing hPSCS to differentiate into mesoderm by contacting the PSCs with a medium comprising an activator of the activin signaling pathway (e.g., an activin) and an activator of a bone morphogenetic protein 4 (BMP4) receptor (e.g., BMP4); and (ii) inducing the mesoderm to differentiate into cardiac progenitors by contacting the mesodermal cells with a Wnt signaling antagonist.

Activins are members of the transforming growth factor beta (TGF-β) family of proteins produced by many cell types throughout development. activin A is a disulfide-linked homodimer (two beta-A chains) that binds to heteromeric complexes of a type I (Act RI-A and Act RI-B) and a type II (Act RII-A and Act RII-B) serine-threonine kinase receptor. activins primarily signal through SMAD2/3 proteins when the activated activin receptor complex phosphorylates the receptor-associated SMAD. The resulting SMAD complex regulates a variety of functions, including cell proliferation and differentiation.

BMPs are part of the transforming growth factor beta superfamily. BMP4 binds to two different types of serine-threonine kinase receptors known as BMPR1 and BMPR2. Signal transduction via these receptors occurs via SMAD and MAP kinase pathways to effect transcription of BMP4's target genes. Various BMPs are suitable for use in generating the cells provided herein, including BMP4 and BMP2.

Wnt signaling antagonists are molecules (e.g., a chemical compound; a nucleic acid, e.g., a non-coding RNA; a polypeptide; and a nucleic acid encoding a polypeptide) that antagonize the Wnt signaling pathway, thus resulting in decreased pathway output (i.e., decreased target gene expression). For example, a Wnt signaling antagonist can function by destabilizing, decreasing the expression of, or inhibiting the function of a positive regulatory component of the pathway, or by stabilizing, enhancing the expression or function of a negative regulatory component of the pathway. Thus, a Wnt signaling antagonist can be a nucleic acid encoding one or more negative regulatory components of the pathway. A Wnt signaling antagonist can also be a small molecule or nucleic acid that stabilizes a negative regulatory component of the pathway at either the mRNA or the protein level. Likewise, a subject Wnt signaling antagonist can be a small molecule or nucleic acid inhibitor (e.g., microRNA, shRNA, etc.) of a positive regulatory component of the pathway that inhibits the component at the mRNA or protein level. In some embodiments, the Wnt signaling antagonist is a small molecule chemical compound (e.g., Xav-939, C59, ICG-001, IWR1, IWP2, IWP4, IWP-L6, pyrvinium, PKF115-584, and the like). In particular embodiments, Wnt antagonism may be achieved by the combined use of Xav-939 and C59 or the combined use of Xav-939 and IWP-L6. See also US20180251734.

For example, to generate cardiac progenitor cells, the PSCs may first be induced to aggregate to form embryoid bodies (EBs). To do so, the PSCs (e.g., hPSCs) may be cultured in an EB medium comprising a BMP component (e.g., BMP4), optionally further comprising a Rho-associated protein kinase (ROCK) inhibitor, for a period of time (e.g., 8-24 hours) to generate embryoid bodies. The EB medium may be made with a Roswell Park Memorial Institute (RPMI) base medium (optionally with B27 supplement), a Dulbecco's Modified Eagle Medium (DMEM) base medium, an Iscove's Modified Dulbecco's Media (IMDM) base medium, or StemPro®-34, with the BMP component and/or ROCK inhibitor added to it. In some embodiments, the concentration of BMP4 in the EB medium is between about 0.1 and 10 ng/ml (e.g., about 0.5-5 ng/ml, or about 0.1, 0.5, 1, 2, 3, 4, 5, 6, 7, 8, 9, or 10 ng/ml). In some embodiments, a ROCK inhibitor (e.g., Y-27632; Biotechne-Tocris #1254) in the EB medium may range in 1-20 μM (e.g., 5-15 μM such as 10 μM). For example, the EB medium may contain 1 ng/ml BMP4 and 10 μM Y-27632.

The EBs may then be cultured in a first differentiation medium (mesoderm induction medium) comprising activin A, BMP4, and optionally fibroblast growth factor-basic (bFGF; also known as basic FGF, FGF-basic, FGF-beta, FGF2, heparin binding growth factor, or FGF family members bind heparin). The mesoderm induction medium may be made with an RPMI base medium (optionally with B27 supplement), a DMEM base medium, an IMDM base medium, or StemPro®-34, with the indicated factors added to it. The selection of activin A, BMP4, and bFGF concentrations may be based on identification of (i) a ventricular mesoderm population that contains a high proportion of ALDH⁻CD235a^{high}) cells or (ii) an atrial mesoderm population that contains a high proportion of ALDH^{high}CD235a^{low}) cells; and generates a high proportion of cTNT cells at day 20. In some embodiments, the concentration of BMP4 in the mesoderm induction media is between about 0.1 and 30 ng/ml (e.g., about 5-10 or 5-15 ng/ml; or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In some embodiments, the mesoderm induction media includes activin A at a concentration of about 0.1 and 30 ng/ml (e.g., about 5-10 or 5-15 ng/ml; or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In some embodiments, the mesoderm induction medium additionally contains 0.1-30 ng/ml bFGF (e.g., about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In particular embodiments, the mesoderm induction medium for generating ventricular cardiomyocytes contains about 8 ng/ml BMP4, about 12 ng/ml activin A, and about 5 ng/ml bFGF. In other particular embodiments, the mesoderm induction medium for generating atrial cardiomyocytes contains about 3 ng/ml BMP4, about 1 ng/ml activin A, and about 5 ng/ml bFGF. The cells may be cultured in the mesoderm induction medium for about 1-3 days (e.g., 1, 1.5, 2, 2.5, or 3 days).

After this culturing step, the cells may be further cultured for at least 1-3 days (e.g., 1, 1.5, 2, 2.5, or 3 days) in a second differentiation medium (cardiac induction medium) comprising a Wnt signaling antagonist, such as IWP2, and optionally comprising VEGF, to generate cardiac progenitor cells. The cardiac induction medium can be made with an RPMI base medium (optionally with B27 supplement), a DMEM base medium, an IMDM base medium, or StemPro®-34, with the indicated factors added to it. In some embodiments, the cardiac induction medium may contain IWP2 at 0.1-10 μM such as 0.5, 1, 2, 3, 4, 5, 6, 7, 8, 9, or 10 μM.

After incubation in the cardiac induction medium, the resultant cardiac progenitor cells may be further cultured for another one to three weeks (e.g., 7-15 days) in a base cardiac medium to obtain a cell population comprising cardiomyocytes. The base cardiac medium may be, for example, an RPMI base medium (optionally with B27 supplement), a DMEM base medium, an IMDM base medium, or StemPro®-34 that is optionally supplemented with VEGF (e.g., at 0.1-30 ng/ml as listed above).

In some embodiments, the EBs are induced to differentiate into cardiac progenitor cells (and eventually cardiomyocytes) in an EB differentiation media, commonly known as EB20 (see, e.g., Lee et al., Circ Res. (2012) 110(12):1556-63). The cardiac progenitors may then be further cultured in a base cardiac medium, such as an RPMI base medium (optionally with B27 supplement), a DMEM base medium, an IMDM base medium, or StemPro®-34, to obtain a cell population comprising human cardiomyocytes (e.g., human cardiac troponinT (cTnT⁺ cells). The base cardiac medium may contain VEGF as described above.

An alternative method for generating human cardiac progenitors from hPSCs (e.g., hESCs and human iPSCs) involves (i) activating Wnt/β-catenin signaling in hPSCs to obtain a first cell population; and (ii) inhibiting Wnt/β-catenin signaling in the first cell population to obtain a second cell population comprising cardiomyocyte progenitors. In some embodiments, small molecules may be used to sequentially activate and inhibit Wnt/β-catenin signaling. Activation of Wnt/β-catenin signaling in hPSCs may be achieved by contacting the hPSCs with a Wnt signaling agonist. In some embodiments, a Wnt signaling agonist functions by stabilizing β-catenin, thus allowing nuclear levels of β-catenin to rise. β-catenin can be stabilized in multiple ways. As multiple negative regulatory components of the Wnt signaling pathway function by facilitating the degradation of β-catenin, a subject Wnt signaling agonist can be a small molecule or nucleic acid inhibitor (e.g., microRNA, shRNA, etc.) of a negative regulatory component of the pathway that inhibits the component at the mRNA or protein level. For example, the Wnt signaling agonist is an inhibitor of glycogen synthase kinase-3β (GSK-3β). In some such embodiments, the inhibitor of GSK-3β is a small molecule chemical compound (e.g., CHIR-99021, TWS119, BIO, SB 216763, SB 415286, CHIR-98014, and the like). Inhibition of Wnt/β-catenin signaling may be achieved by contacting the cells that were previously contacted with the Wnt signaling agonist, with a Wnt signaling antagonist, such as those described above. In general, after ending the inhibition of Wnt/β-catenin signaling, cardiac progenitors may be further cultured in a base cardiac medium, such as an RPMI base medium (optionally with B27 supplement), a DMEM base medium, an IMDM base medium, or StemPro®-34, to obtain a cell population comprising human cardiomyocytes (e.g., human cardiac troponinT (cTnT⁺ cells).

In some embodiments, the starting population is cardiovascular mesoderm cells. Such cells, express surface markers PDGRF-alpha(high) and KDR(low) (U.S. Pat. No. 10,561,687). In addition, these cells express surface marker CD56 and express MESP1 and T(Brachyury) by Q-RT-PCR, and can give rise to cTNT+ cardiomyocytes. Addition of FGF inhibitor to cardiovascular mesoderm cells dramatically increases the proportion of sinoatrial node-like cardiomyocytes when assessed at day 20 of culture (ibid). Accordingly, in some embodiments, the cardiovascular mesoderm cells are also treated with an FGF inhibitor for all or part of the cardiac induction phase.

The medium used at the various differentiation stages as described above can be made with any suitable base medium, which includes, without limitation, an RPMI base medium (optionally with B27 supplement), a DMEM base medium, an IMDM base medium, or StemPro®-34, with the indicated factors (e.g., cytokines and small molecules) added to the base medium.

In some embodiments, for optimal ventricular or atrial inductive conditions, the PSCs may be incubated in a cardiac differentiation media containing activin A and bone morphogenic protein 4 (BMP4). See, e.g., Lee, supra. To generate ventricular cardiomyocytes, the selection of activin A and BMP4 concentrations may be based on identification of a mesoderm population that contains a high proportion of CD235a⁺ cells, no ALDH⁺ cells and generates a high proportion of cTNT⁺MLC2V⁺ at day 20. To generate atrial cardiomyocytes, the selection of activin A and BMP4 concentrations may be based on identification of a mesoderm population that contains a high proportion of ALDH⁺ cells, no CD235a⁺ cells at day 4 and generates a high proportion of cTNT⁺MLC2V⁻ at day 20. In some embodiments, the concentration of BMP4 in the differentiation media is between about 1 and 30 ng/ml (e.g., about 3-10 or 3-15 ng/ml; or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In some embodiments, the differentiation media includes activin A at a concentration of about 1 and 30 ng/ml (e.g., about 5-10 or 5-15 ng/ml; or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In particular embodiments, the differentiation media contains about 10 ng/ml BMP4 and about 6 ng/ml activin A. In other particular embodiments, the differentiation medium contains about 8 ng/ml BMP4 and about 12 ng/ml activin A. In some other particular embodiments, the differentiation medium contains about 3 ng/ml BMP4 and about 1 ng/ml activinA. During the ventricular or atrial cardiomyocyte differentiation process, the PSCs may be incubated with a differentiation media containing BMP4 and activin A for about 1-3 days (e.g., 1, 1.5, 2, 2.5, or 3 days). During the atrial cardiomyocyte differentiation process, the PSCs may be further incubated with a differentiation medium containing about 0.1-2 μM (e.g., 0.5 μM) all-trans Retinoic Acid (ATRA) or about 0.5-4 μM (e.g., 2 μM Retinol) from about day 3 to day 5 as previously described in Lee, supra. See also Examples 1-4 below for exemplary, nonlimiting cardiac differentiation protocols that may be used herein.

The above protocols may be adapted for scaled up production of large quantities of cardiac progenitors and/or cardiomyocytes. For example, bioreactors, large roller bottles, and other culturing devices may be used in lieu of multi-well tissue culture plates.

### Specification of Ventricular Cardiomyocytes to the Compact Lineage

The present disclosure provides methods of promoting specification of ventricular cardiomyocyte progenitor cells to the compact lineage during the differentiation of PSCs to ventricular cardiomyocytes. As used herein, ventricular cardiomyocyte progenitor cells, or ventricular cardiomyocyte precursor cells, are cells that have committed to a ventricular cardiomyocyte fate but have not yet committed fully to a particular ventricular subtype (e.g., the compact or trabecular subtype). Ventricular cardiomyocytes of the compact lineage, or “ventricular compact cardiomyocytes,” are characterized by being HEY2⁺MYCN⁺ANF⁻ (HEY2: Hes-related family BHLH transcription factor with YRPW motif 2; MYCN: n-myc proto-oncogene protein; ANF: atrial natriuretic factor). In some embodiments, ventricular compact cardiomyocytes are characterized by HEY2^{high}ANF⁻, wherein “high” means that the expression level of HEY2 is three or more times higher than the expression level of reference protein TBP in the cells.

To promote the specification of ventricular cardiomyocyte progenitor cells to the compact lineage, so as to obtain a cell population enriched for ventricular compact cardiomyocytes, factors that activate the Wnt/β-catenin pathway (a “Wnt signaling agonist”) and a cell proliferation stimulator (e.g., IGF2, IGF1, or insulin) may be used.

As used herein, a Wnt signaling agonist is any molecule (e.g., a chemical compound; a non-coding nucleic acid, e.g., a non-coding RNA; a polypeptide; and a nucleic acid encoding a polypeptide) that results in increased output (i.e., increased target gene expression) from the Wnt signaling pathway. For example, a Wnt signaling agonist can function by stabilizing or enhancing the expression or function of a positive regulatory component of the pathway, or by destabilizing, decreasing the expression of, or inhibiting the function of a negative regulatory component of the pathway. Thus, a Wnt signaling agonist can be a nucleic acid encoding one or more positive regulatory components of the pathway. A Wnt signaling agonist can also be a small molecule or nucleic acid that stabilizes a positive regulatory component of the pathway at either the mRNA or the protein level.

In some embodiments, a Wnt signaling agonist functions by stabilizing β-catenin, thus allowing nuclear levels of β-catenin to rise. β-catenin can be stabilized in multiple ways. As multiple negative regulatory components of the Wnt signaling pathway function by facilitating the degradation of β-catenin, a subject Wnt signaling agonist can be a small molecule or nucleic acid inhibitor (e.g., microRNA, shRNA, etc.) of a negative regulatory component of the pathway that inhibits the component at the mRNA or protein level. For example, the Wnt signaling agonist is an inhibitor of glycogen synthase kinase-3β (GSK-3β). In some such embodiments, the inhibitor of GSK-3β is a small molecule chemical compound (e.g., CHIR-99021, TWS119, BIO, SB 216763, SB 415286, CHIR-98014, and the like).

In some embodiments, the ventricular cardiomyocyte progenitor cells, such as those cells being differentiated from PSCs to ventricular cells as described above, are treated with CHIR-99021 (a Wnt signaling agonist) and IGF2. CHIR-99021 (“CHIR” herein) is 6-((2-((4-(2,4-Dichlorophenyl)-5-(4-methyl-1H-imidazol-2-yl)pyrimidin-2-yl)amino)ethyl)amino)nicotinonitrile, with the following chemical structure:

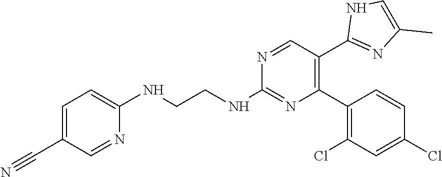 [SMILES: Cc1c[nH]c(-c2cnc(NCCNc3ccc(C#N)cn3)nc2-c2ccc(Cl)cc2Cl)n1]

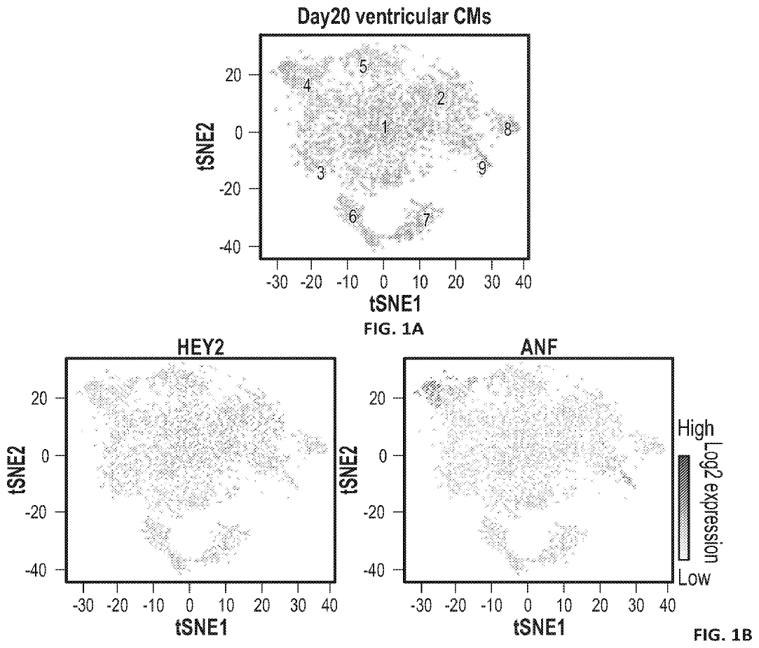

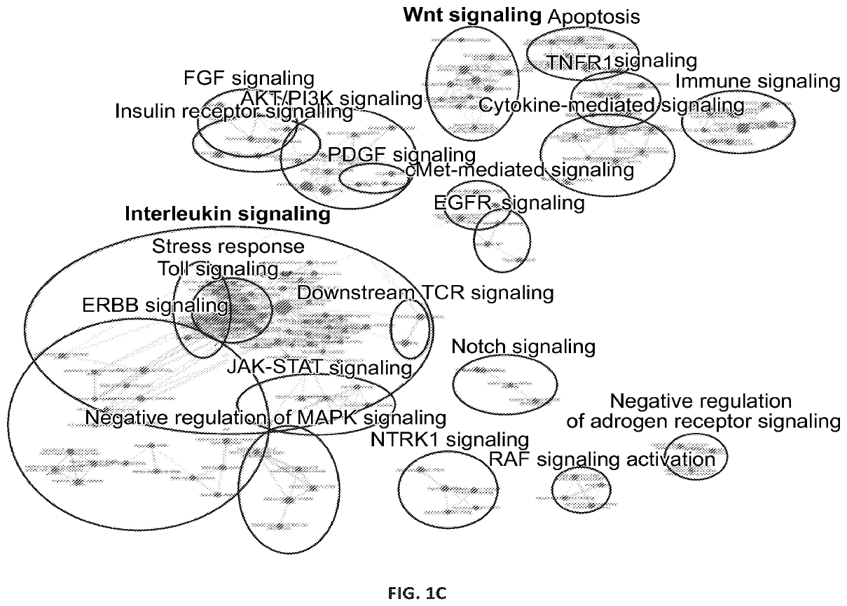

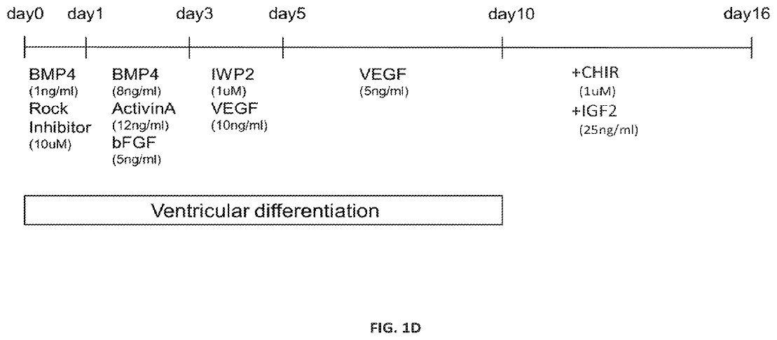

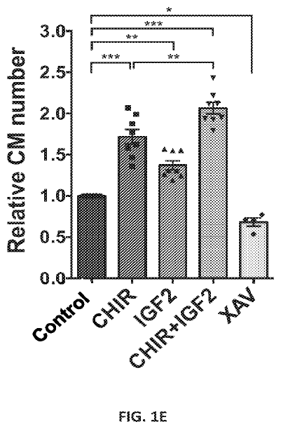

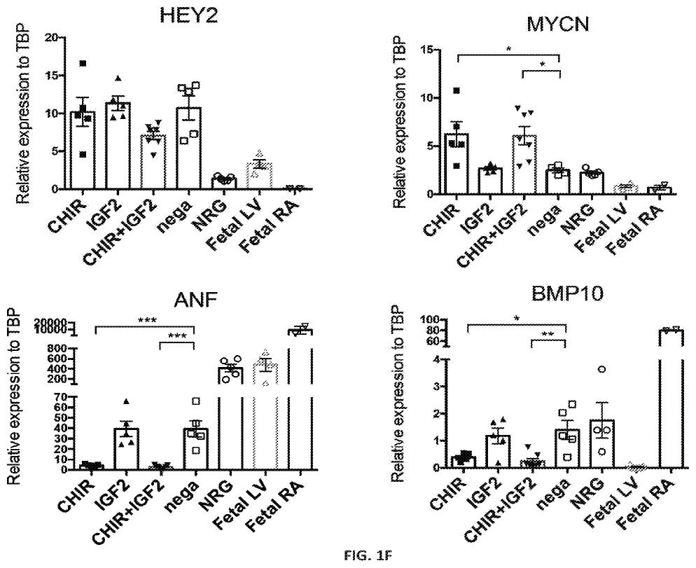

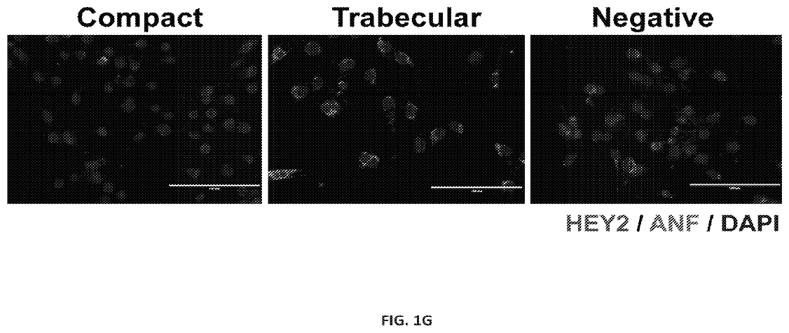

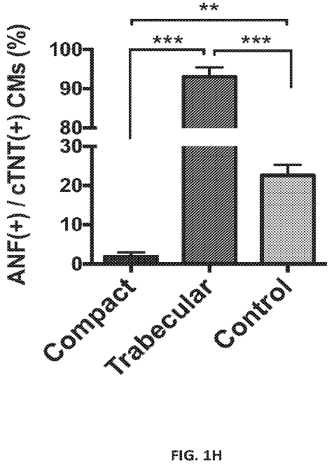

The selection of CHIR and IGF2 concentrations may depend on factors such as the differentiation status of the cells and cell culture conditions. Their concentrations may be optimized to achieve the highest number of ventricular cells of the compact lineage as characterized by being HEY2⁺MYCN⁺ANF⁻. In some embodiments, the concentration of CHIR in the differentiation media is between about 0.1-10 μM (e.g., 0.5, 1, 2, 3, 4, or 5 μM). In some embodiments, the differentiation media includes IGF2 at a concentration of about 1 and 50 ng/ml (e.g., 1, 5, 10, 15, 20, 25, 30, 35, 40, 45, or 50 ng/ml). In particular embodiments, the differentiation medium contains about 1 μM CHIR and about 25 ng/ML IGF2. During the compact specification process, the cells may be incubated with the differentiation medium 1 day to one week, e.g., for 6 or 7 days. An exemplary, nonlimiting protocol is shown in FIG. 1D.

### Generation of Mature Cardiomyocytes

The present disclosure further provides methods of promoting the metabolic maturation of the in vitro derived ventricular (e.g., compact) or atrial cardiomyocytes. Molecular and metabolic analyses provided herein have revealed that the FAO pathway, rather than glycolysis, is active in mature ventricular (e.g., compact) or atrial cardiomyocytes and that these cells can efficiently use exogenous fatty acids as well as stored lipid reserves as an energy source. These cells contain structurally mature mitochondria, and display organized sarcomere structures and mature electrophysiological properties such as increased conduction velocity. Metabolically mature cells represent ideal target cells for cell-based therapy and disease modeling.

To promote the metabolic maturation of the in vitro derived ventricular or atrial cardiomyocytes, the cultured cells may be treated with a cell proliferation inhibitor to slow down cell proliferation. The cell proliferation inhibitor may be, for example, an inhibitor of the Wnt/β-catenin pathway (a “Wnt signaling antagonist”).

As used herein, a Wnt signaling antagonist is any molecule (e.g., a chemical compound; a nucleic acid, e.g., a non-coding RNA; a polypeptide; and a nucleic acid encoding a polypeptide) that antagonizes the Wnt signaling pathway, thus resulting in decreased pathway output (i.e., decreased target gene expression). For example, a Wnt signaling antagonist can function by destabilizing, decreasing the expression of, or inhibiting the function of a positive regulatory component of the pathway, or by stabilizing, enhancing the expression or function of a negative regulatory component of the pathway.

Thus, a Wnt signaling antagonist can be a nucleic acid encoding one or more negative regulatory components of the pathway. A Wnt signaling antagonist can also be a small molecule or nucleic acid that stabilizes a negative regulatory component of the pathway at either the mRNA or the protein level. Likewise, a subject Wnt signaling antagonist can be a small molecule or nucleic acid inhibitor (e.g., microRNA, shRNA, etc.) of a positive regulatory component of the pathway that inhibits the component at the mRNA or protein level. In some embodiments, the Wnt signaling antagonist is a small molecule chemical compound (e.g., Xav-939, C59, ICG-001, IWR-1, IWP-2, IWP-4, pyrvinium, PKF115-584, and the like).

In some embodiments, the cell proliferation inhibitor is the Wnt signaling antagonist Xav-939 (“XAV” herein) is 2-(4-(trifluoromethyl)phenyl)-7,8-dihydro-5H-thiopyrano[4,3-d]pyrimidin-4-ol, with the following chemical structure.

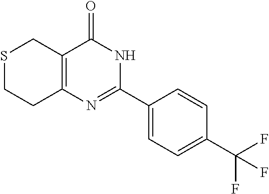 [SMILES: O=c1[nH]c(-c2ccc(C(F)(F)F)cc2)nc2c1CSCC2]

The cultured cells, such as those that have been specified or in the process of being specified to the compact lineage as described above, may be treated with XAV for 1-5 days (e.g., one or two days). In some embodiments, the culture medium contains about 0.1-10 μM (e.g., 1, 2, 3, 4, or 5 μM) XAV. In particular embodiments, the cells to be matured are cultured with 4 μM XAV for two days.

Concurrent with or subsequent to the treatment with the cell proliferation inhibitor, the ventricular or atrial cardiomyocytes can be treated with a combination of factors that promote the cells' metabolic switch from glycolysis to FAO. In some embodiments, the culture medium contains a peroxisome proliferator-activated receptor alpha (PPARα) agonist, a hydrocortisone (e.g., dexamethasone), and a thyroid hormone (e.g., triiodothyronine (T3), 3, 5-diiodothyropropionic acid (DITPA), or sobetirome).

As used herein, a PPARα agonist is any molecule (e.g., a chemical compound; a nucleic acid, e.g., a non-coding RNA; a polypeptide; and a nucleic acid encoding a polypeptide) that activates PPARα. In some embodiments, the PPARα agonist is a small molecule chemical compound (e.g., GW7647, CP775146, fenofibrate, oleylethanolamide, palmitoylethanolamide, WY14643, and the like). In some embodiments, the PPARα agonist is GW7647. GW7647 is 2-(4-(2-(1-cyclohexanebutyl)-3-cyclohexylureido)ethyl)phenylthio)-2-methylpropionic acid, with the following chemical structure.

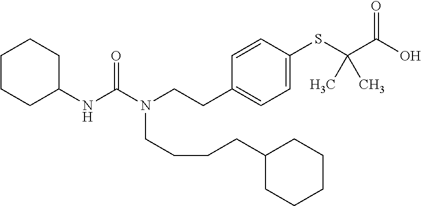 [SMILES: CC(C)(Sc1ccc(CCN(CCCCC2CCCCC2)C(=O)NC2CCCCC2)cc1)C(=O)O]

In some embodiments, the maturation culture medium contains GW7647, dexamethasone, and thyroid hormone T3. In further embodiments, the culture medium contains 0.1-10 μM (e.g., 0.5, 1, 1.5, or 2 μM) GW7647, 10-200 ng/ml (e.g., 50, 100, or 150 ng/ml) dexamethasone, and 1-10 nM (e.g., 1, 2, 4, or 8 nM) thyroid hormone T3. The ventricular (e.g., compact) or atrial cardiomyocytes to be matured are cultured in the maturation medium for 1-3 weeks (e.g., 7 days, 14 days, or 21 days). In particular embodiments, the cells are cultured for 14 days in a culture medium containing 1 μM GW7647, 100 ng/ml dexamethasone, and 4 nM thyroid hormone T3.

In some embodiments, the maturation medium may also contain fatty acids (e.g., long-chain fatty acids such as those with 16 or more carbons) and/or glucose. For example, the culture medium may contain 50-500 μM (e.g., 100, 150, 200, or 250 μM) palmitate and optionally a low concentration of glucose (e.g., 1-2 mg/ml). 4 mg/ml glucose is considered a high glucose concentration.

It may be advantageous that during the culturing process, the cell culture container is agitated, for example by stirring, rotating, rocking, and/or shaking, so as to, e.g., reduce clumping of cells.

In particular embodiments, the cells to be matured are cultured for 14 days in a culture medium containing 1 μM GW7647, 100 ng/ml dexamethasone, 4 nM thyroid hormone T3, 200 μM palmitate, and 2 mg/ml glucose, while the cell culture container is rotated at a speed of 50-100 rpm (e.g., 50, 60, 70, 80, 90, or 100 rpm).

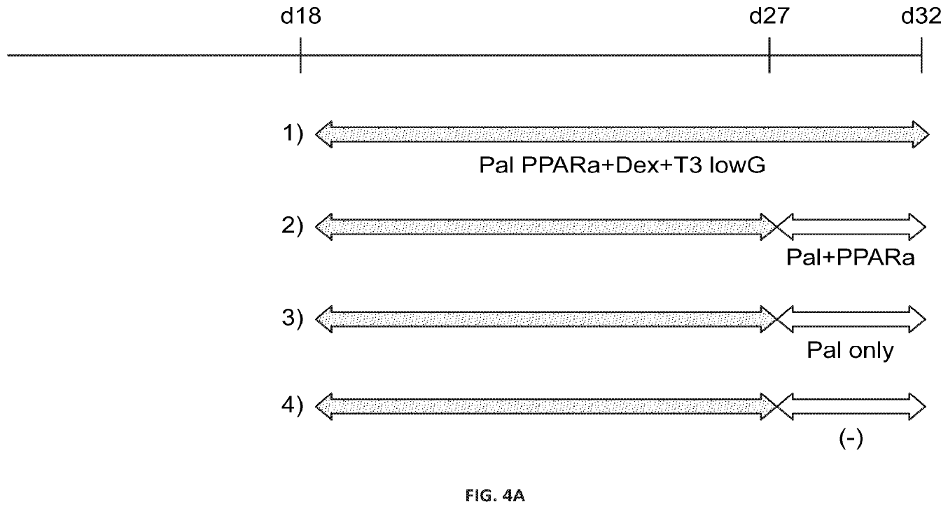

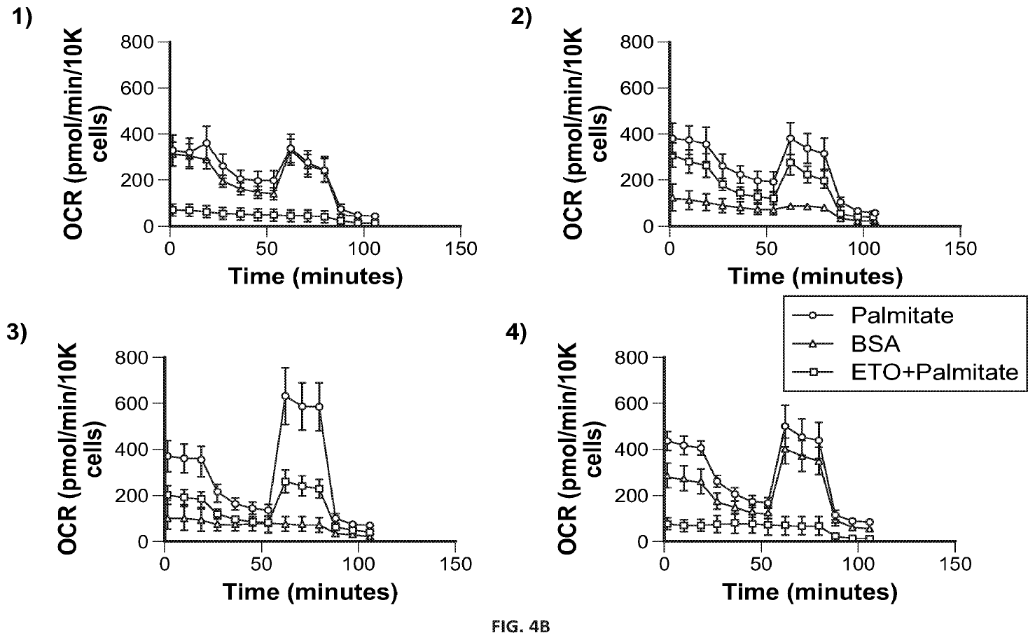

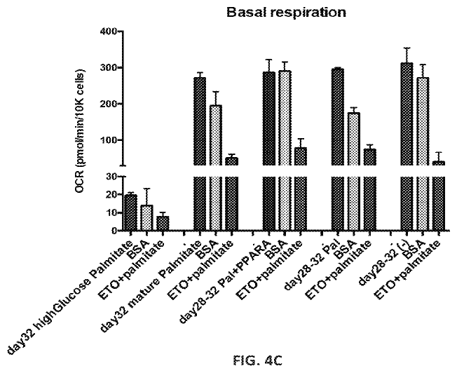

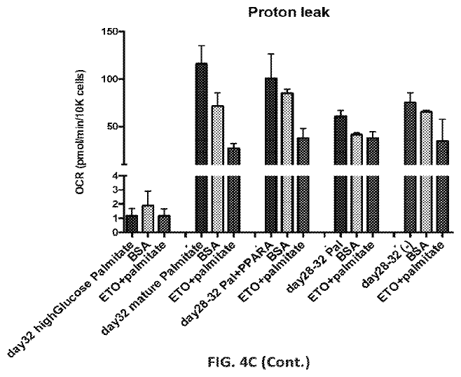

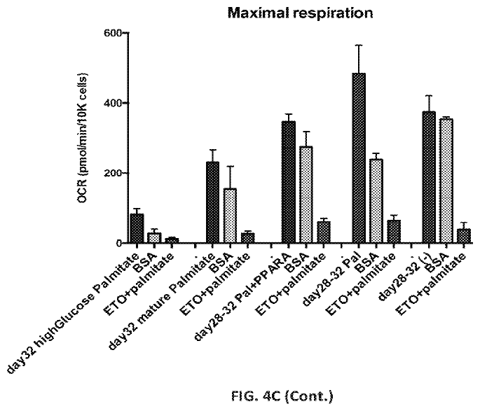

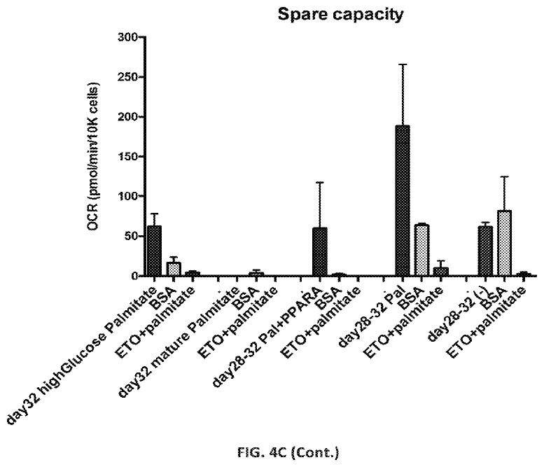

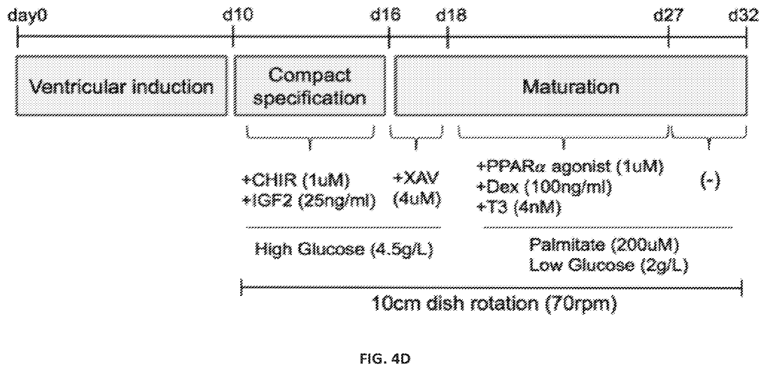

In an exemplary, nonlimiting embodiment, human PSCs are cultured in the presence of BMP4 and activin A for about 2 days, then cultured in the presence of a Wnt inhibitor (e.g., IWP2 at, e.g., 1 mM) and VEGF (e.g., 10 ng/mL) for about 2 days, then cultured in the presence of VEGF (e.g., 5 ng/mL) for about 5 days, then cultured in the presence of CHIR (e.g., 1 μM) and IGF2 (e.g., 25 ng/ml) for about 6 days, then cultured in the presence of XAV (e.g., 4 μM) for 2 days, and then cultured in the presence of 1 μM GW7647, 100 ng/ml dexamethasone, 4 nM thyroid hormone T3, 200 μM palmitate, and 2 mg/ml glucose for 14 days, where the cell culture container is rotated at a speed of 50-100 rpm (e.g., 50, 60, 70, 80, 90, or 100 rpm). See also FIG. 4D.

### Further Enrichment of Mature Cardiomyocytes

The above-described culture methods may lead to a ventricular or atrial cardiomyocyte population enriched for (e.g., more than 50%, more than 60%, or more than 70%) metabolically mature ventricular or atrial, respectively, cardiomyocytes that display the capacity to use fatty acids as an energy source. As exemplified below, these mature cells display gene expression patterns and structural and electrophysiological properties of human neonatal cardiomyocytes. To further enrich for mature cardiomyocytes (to achieve a purity more than 80%, and up to 99%), an important requirement for cardiac cell therapy, the present inventors have unexpectedly discovered unique gene signatures (including gene expression analysis at the transcript or protein level) that help identify mature ventricular compact cardiomyocytes. Mature ventricular compact cardiomyocytes may be characterized by MLC2V⁺HEY2⁺ANF⁻BMP10⁻. Mature ventricular compact cardiomyocytes also may express high levels of one or more (e.g., two or more, three or more, four or more, five or more, six or more, seven or more, eight or more, nine or more, or ten or more) of the following genes: CD36; low density lipoprotein receptor (LDLR); fatty acid binding protein 3 (FABP3); cytochrome C oxidase subunit 6A2 (COX6A2); ATP synthase F1 subunit alpha (ATP5A1); cytochrome C oxidase subunit 7A1 (COX7A1); creatine kinase, mitochondrial 2 (CKMT2); superoxide dismutase 2 (SOD2); ankyrin repeat and SOCS box containing 2 (ASB2); fibroblast growth factor 12 (FGF12); carnitine palmitoyltransferase 1B (CPT1B); malonyl-CoA decarboxylase (MLYCD); pyruvate dehydrogenase kinase 4 (PDK4); titin-cap (TCAP); potassium inwardly rectifying channel subfamily J member 2 (KCNJ2); ATPase sarcoplasmic/endoplasmic reticulum Ca²⁺ transporting 2 (ATP2A2); adrenoceptor beta 1 (ADRB1); uncoupling protein 2 (UCP2); uncoupling protein 3 (UCP3); tumor protein p53 inducible nuclear protein 2 (TP53INP2); nicotinamide riboside kinase 2 (NMRK2); natriuretic peptide B (NPPB); heat shock protein family B (small) member 6 (HSPB6); Kruppel like factor 9 (KLF9); CCAAT enhancer binding protein beta (CEBPB); mannan binding lectin serine peptidase 1 (MASP1); histidine rich calcium binding protein (HRC); long-chain acyl-CoA synthetase 1 (ACSL1); estrogen related receptor alpha (ESRRA); and stearoyl-CoA desaturase (SCD). Expression of these genes may be assessed at the mRNA level (e.g., by quantitative RT-PCR), or at the protein level (e.g., by cell surface staining or staining of a permeabilized cell by an antibody). A high expression level refers to a level higher (e.g., 1.5, 2, 3, 4, 5, or 10 fold higher) than a control level (e.g., a level in an immature counterpart).

In some embodiments, mature ventricular compact cardiomyocytes can be characterized by one, more, or all of CD36, CPT1B, MLYCD, PDK4, TCAP, KCNJ2, ATP2A2, ADRB1, UCP2, and UCP3.

In some embodiments, mature ventricular compact cardiomyocytes can be characterized by one, more, or all of CD36, LDLR, FABP3, ACSL1, COX6A2, ATP5A1, COX7A1, CKMT2, SOD2, ASB2, FGF12, CPT1B, MLYCD, PDK4, TCAP, KCNJ2, ATP2A2, ADRB1, UCP2, and UCP3.

In some embodiments, mature ventricular compact cardiomyocytes can be characterized by one, more, or all of CD36, NMRK2, NPPB, HSPB6, KLF9, CEBPB, MASP1, HRC, ACSL1, ESRRA, and SCD.

In some embodiments, mature ventricular compact cardiomyocytes can be characterized by one, more, or all of CD36, NMRK2, NPPB, HSPB6, KLF9, CEBPB, and ESRRA.

In some embodiments, mature ventricular compact cardiomyocytes can be characterized by one, more, or all of CD36, NMRK2, NPPB, HSPB6, MASP1, HRC, ACSL1, and SCD.

In some embodiments, mature ventricular compact cardiomyocytes can be characterized by CD36, LDLR, and NMRK2; CD36 and NMRK2; or CD36 and LDLR.

Since CD36 and LDLR are cell surface markers, they are particularly useful for quantifying the proportion of CD36⁺LDLR⁺ mature ventricular compact cardiomyocytes in a cell culture and isolating these cells from the cell culture. CD36/LDLR expression can thus be used as a basis for evaluating the purity of a mature ventricular compact cardiomyocyte composition, for example, as part of quality control for cardiac cell therapy products.

Phenotypically, mature ventricular compact cardiomyocytes may also be characterized by increased (a) mitochondria mass, (b) sarcomere length, (c) conduction velocity, and/or (d) contractile force, as compared to immature ventricular compact cardiomyocyte.

Isolation of mature ventricular or atrial cardiomyocytes from cell cultures may be accomplished using sorting methods available in the art that include, but are not limited to, fluorescence-activated cell sorting (FACS), magnetic-activated cell sorting (MACS) (e.g., immunomagnetic cell sorting), buoyancy-activated cell sorting (BACS™) (see, e.g., Lio et al., PLoS One (2015) 10(5):e0125036), and the like. Combinations of cell sorting methods may also be employed, as necessary, to reduce sort time and improve purity and recovery. These methods may be further combined with methods that measure the temporal expression of CD36 or LDLR during the cardiomyocyte cell culture differentiation process to determine the optimal time for selection of the mature ventricular compact and/or atrial cardiomyocyte. Antibodies or antigen-binding fragment thereof (e.g., scFv, scFv-Fc fusion, Fab, or F(ab′)2), or other binding moieties (e.g., fusion proteins) that bind the target cell markers may be used.

### Pharmaceutical Compositions and Use

The present highly enriched populations of ventricular compact and/or atrial cardiomyocytes and the present enriched populations of mature ventricular compact and/or mature atrial cardiomyocytes can be used in cell therapy to treat a subject (e.g., a human subject) with cardiomyopathy or at risk of having cardiomyopathy. Cardiomyopathy is a group of conditions including, without limitation, ischemic heart disease, myocardial infarction (acute and chronic), left ventricular heart failure, right ventricular heart failure, myocarditis (e.g., myocarditis caused by bacterial or viral infection), dilated cardiomyopathy, and congenital heart disease. In some embodiments, the heart failure is left-sided heart failure, a right-sided heart failure, a diastolic heart failure, or a systolic heart failure. In some embodiments, the heart failure is congestive heart failure. In some embodiments, the subject is suffering from one or more previous myocardial infarctions. In further embodiments, the one or more myocardial infarctions are in the ventricle (e.g., left ventricle) of the subject. The cell therapy provided herein results in repair of cardiac muscle and restoration of cardiac function in the subject, thus treating the cardiomyopathy.

The present cell preparations can treat cardiomyopathy by: (1) repopulating diseased (e.g., scarred) myocardium with contractile myocytes; (2) providing a scaffolding to diminish further abnormal remodeling of the thinned, injured ventricle; and/or (3) serving as a vehicle for the release of salutary paracrine factors such as pro-angiogenic, cardioprotective, matrix-remodeling or anti-inflammatory signals. Due to the purity of the present cell preparations, the present cell therapy will result in fewer side effects, including less frequent ventricular arrhythmias as compared to prior cell therapy.

The cell preparations of the present disclosure may be administered systemically or transplanted locally into a subject in need thereof. Various methods are known in the art for administering cells into a patient's heart, for example, intracoronary administration, intramyocardial administration, or transendocardial administration. By way of example, the cells can be introduced to the heart by using a catheter inserted via the femoral, subclavian, jugular or axillary vein, or by endocardial transplantation into the ventricle or atrium region. The cells also can be transplanted into the ventricle or atrium region by an epicardial approach, using a needle inserted through the chest. Fluoroscopy (X-ray based method) or 3D mapping can be used to guide the catheter/needle to the intended injection site.

The enriched or highly enriched cell populations described herein may be provided in a pharmaceutical composition containing the cells and a pharmaceutically acceptable carrier. In some embodiments, the pharmaceutical composition comprises a population of PSC-derived ventricular compact cardiomyocytes as described herein and a pharmaceutically acceptable carrier and/or additives. In some embodiments, the pharmaceutical composition comprises a population of PSC-derived mature ventricular compact or mature atrial cardiomyocytes as described herein and a pharmaceutically acceptable carrier and/or additives. For example, a cell culture medium (e.g., one that optionally does not contain any animal-derived component), sterilized water, physiological saline, general buffers (e.g., phosphoric acid, citric acid, other organic acids, etc.), stabilizers, salts, anti-oxidants, surfactants, suspensions, isotonic agents, and/or preservatives may be included in the pharmaceutical composition. In some embodiments, the pharmaceutical composition is formulated into a dosage form suitable for administration to a subject in need of treatment. In some embodiments, the pharmaceutical composition is formulated into a dosage form suitable for intramyocardial administration, transendocardial administration, or intracoronary administration. For storage and transportation, the cells optionally may be cryopreserved. Prior to use, the cells may be thawed and diluted in a sterile carrier that is supportive of the cell type of interest.

A therapeutically effective number of ventricular compact or atrial cardiomyocytes and/or mature ventricular compact or atrial cardiomyocytes are administered to the patient. As used herein, the term “therapeutically effective” refers to a number of cells or amount of pharmaceutical composition that is sufficient, when administered to a human subject suffering from or susceptible to a disease, disorder, and/or condition, to treat, prevent, and/or delay the onset or progression of the symptom(s) of the disease, disorder, and/or condition. It will be appreciated by those of ordinary skill in the art that a therapeutically effective amount is typically administered via a dosing regimen comprising at least one unit dose.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

### Exemplary Embodiments

Exemplary, nonlimiting embodiments of some aspects of the present disclosure are described as follows. These embodiments are intended to illustrate the compositions and methods described in the present disclosure and are not intended to limit the scope of the present disclosure.

1. A method of promoting differentiation of a ventricular cardiomyocyte progenitor cell into a ventricular compact cardiomyocyte, comprising contacting the progenitor cell with a Wnt signaling agonist and a cell proliferation stimulator, thereby obtaining a ventricular compact cardiomyocyte characterized by being HEY2⁺ ANF⁻.
2. The method of embodiment 1, wherein the cardiomyocyte progenitor cell is derived from a human pluripotent stem cell (PSC).
3. The method of embodiment 2, wherein the human PSC is an induced human PSC or a human embryonic stem cell.
4. The method of any one of the preceding embodiments, wherein the Wnt signaling agonist is an inhibitor of glycogen synthase kinase-3β (GSK-3β).
5. The method of embodiment 4, wherein the inhibitor of GSK-3β is selected from CHIR-99021, TWS119, BIO, SB 216763, SB 415286, and CHIR-98014.
6. The method of embodiment 5, wherein the inhibitor of GSK-3β is CHIR-99021.
7. The method of any one of the preceding embodiments, wherein the cell proliferation stimulator is insulin-like growth factor 2 (IGF2).
8. The method of embodiment 7, wherein the progenitor cell is contacted with CHIR-99021 at 0.1-10 μM and IGF2 at 1-50 ng/ml for 1-7 days.
9. The method of embodiment 8, wherein the progenitor cell is contacted with CHIR-99021 at about 1 μM and IGF2 at about 25 ng/ml for about six days.
10. A plurality of ventricular compact cardiomyocytes obtained by the method of any one of the preceding embodiments.
11. A pharmaceutical composition consisting of a cellular component and a carrier component, wherein the cellular component is a cell population in which more than 80% of the cells are ventricular compact cardiomyocytes characterized by being HEY2⁺MYCN⁺ANF⁻, and wherein the carrier component comprises a pharmaceutically acceptably carrier.
12. A method of promoting metabolic maturation of a ventricular compact cardiomyocyte, comprising contacting a ventricular compact cardiomyocyte with a PPARα signaling agonist, a hydrocortisone, and a thyroid hormone, thereby obtaining a mature ventricular compact cardiomyocyte characterized by being MLC2V⁺HEY2⁺ ANF⁻ and further characterized by one or more of the following features: i) expressing, optionally at a high level, one or more of cellular markers selected from CD36, CPT1B, MLYCD, PDK4, TCAP, KCNJ2, ATP2A2, ADRB1, UCP2, and UCP3; and ii) increased (a) mitochondria mass, (b) sarcomere length, and/or (c) conduction velocity, compared to immature ventricular compact cardiomyocyte.
13. The method of embodiment 12, wherein the PPARα signaling agonist is selected from GW7647, CP775146, fenofibrate, oleylethanolamide, palmitoylethanolamide, and WY14643.
14. The method of embodiment 13, wherein the PPARα signaling agonist is GW7647.
15. The method of any one of embodiments 12-14, wherein the hydrocortisone is dexamethasone.
16. The method of any one of embodiments 12-15, wherein the thyroid hormone is T3.
17. The method of any one of embodiments 12-16, further comprising contacting the ventricular compact cardiomyocyte with a fatty acid containing 16 or more carbons.
18. The method of embodiment 17, wherein the fatty acid is palmitate or a derivative thereof.
19. The method of any one of embodiments 12-18, further comprising culturing the ventricular compact cardiomyocyte in a culture medium containing glucose.
20. The method of embodiment 19, comprising culturing the ventricular compact cardiomyocyte in a culture medium containing GW7647, dexamethasone, thyroid hormone T3, palmitate, and glucose for a period of about one to three weeks.
21. The method of embodiment 20, comprising culturing the ventricular compact cardiomyocyte in a culture medium containing about 1 μM GW7647, about 100 ng/ml dexamethasone, about 4 nM thyroid hormone T3, about 200 μM palmitate, and about 2 mg/ml glucose for a period of about two weeks, wherein the culture medium is agitated during the culturing step.
22. A plurality of mature ventricular compact cardiomyocytes obtained by the method of any one of embodiments 12-21.
23. A pharmaceutical composition consisting of a cellular component and a carrier component, wherein the cellular component is a cell population in which more than 50% of the cells are mature ventricular compact cardiomyocytes, and wherein the carrier component comprises a pharmaceutically acceptable carrier, wherein the mature ventricular compact cardiomyocytes are characterized by being MLC2V⁺HEY2⁺ANF⁻ and further characterized by one or more of the following features: i) expressing, optionally at a high level, one or more of cellular markers selected from CD36, CPT1B, MLYCD, PDK4, TCAP, KCNJ2, ATP2A2, ADRB1, UCP2, and UCP3; and ii) increased (a) mitochondria mass, (b) sarcomere length, and/or (c) conduction velocity, compared to immature ventricular compact cardiomyocyte.
24. A method of generating a cell population enriched for mature ventricular compact cardiomyocytes, comprising

contacting a ventricular cardiomyocyte progenitor cell with a Wnt signaling agonist and a cell proliferation stimulator, thereby obtaining a first cell population enriched for ventricular compact cardiomyocytes characterized by being HEY2⁺MYCN⁺ANF⁻, wherein the Wnt signaling agonist is optionally CHIR-99021 and the cell proliferation stimulator is optionally insulin-like growth factor 2 (IGF2),

contacting the first cell population with a Wnt signaling antagonist, and

culturing the contacted first cell population in the presence of a PPARα signaling agonist, dexamethasone, and thyroid hormone T3, thereby obtaining a second cell population enriched for mature ventricular compact cardiomyocyte characterized by being MLC2V⁺HEY2⁺MYCN⁺ANF⁻ and further characterized by one or more of the following features: i) expressing, optionally at a high level, one or more of cellular markers selected from CD36, CPT1B, MLYCD, PDK4, TCAP, KCNJ2, ATP2A2, ADRB1, UCP2, and UCP3; and ii) increased (a) mitochondria mass, (b) sarcomere length, and/or (c) conduction velocity, compared to immature ventricular compact cardiomyocyte.

25. The method of embodiment 24, wherein the Wnt signaling antagonist is Xav-939.
26. The method of embodiment 24, comprising

contacting the cardiomyocyte progenitor cell with CHIR-99021 at about 1 μM and IGF2 at about 25 ng/ml for about six days to obtain the first cell population,

contacting the first cell population with about 4 μM Xav-939 for about two days, and

culturing the contacted first cell population in a culture medium containing about 1 μM GW7647, about 100 ng/ml dexamethasone, about 4 nM thyroid hormone T3, about 200 μM palmitate, and about 2 g/L glucose for a period of about two weeks, wherein the cell culture is agitated during the culturing step.

27. A plurality of mature ventricular compact cardiomyocytes obtained by the method of any one of embodiments 24-26.
28. A method of treating a cardiomyopathy condition in a subject in need thereof, comprising administering to the subject the plurality of cells of embodiment 10, 22, or 27, or the pharmaceutical composition of embodiment 11 or 23.
29. A plurality of cells of embodiment 10, 22, or 27, or the pharmaceutical composition of embodiment 11 or 23 for use in treating a cardiomyopathy condition.
30. Use of the plurality of cells of embodiment 10, 22, or 27 in the manufacture of a medicament for treating a cardiomyopathy condition.
31. The method of embodiment 28, the cells or composition for use of embodiment 29, or the use of embodiment 30, wherein the cardiomyopathy is myocardial infarct.

In order that this invention may be better understood, the following examples are set forth. These examples are for purposes of illustration only and are not to be construed as limiting the scope of the invention in any manner.

### EXAMPLES

The materials and methods for the experiments described in the following working examples are set forth below.

### Flowcytometry

The EBs were dissociated by incubation in Collagenase type 2 (0.5 mg/ml, Worthington) in HANKs buffer overnight at room temperature followed by TrypLE™ for 5 mins at 37° C. The following antibodies were used for staining: anti-SIRPa-PeCy7 (Biolegend, 1:1000), anti-CD36-FITC (BD PharMingen™, 1:200), anti-LDLR-BV421 (BD PharMingen™, 1:100), anti-cardiac isoform of cTNT (ThermoFisher Scientific, 1:2000), or anti-myosin light chain 2 (Abcam,1:1000). For unconjugated primary antibodies, the following secondary antibodies were used for detection: goat anti-mouse IgG-APC (BD PharMingen™, 1:500), or donkey anti-rabbit IgG-PE (Jackson ImmunoResearch, 1:500). Detailed antibody information is described in the Key Resources Table. For cell-surface marker analyses, cells were stained for 30 min at 4° C. in FACS buffer consisting of PBS with 5% fetal calf serum (FCS) (Wisent) and 0.02% sodium azide. For intracellular staining, cells were fixed for 20 mins at 4° C. with 4% PFA in PBS followed by permeabilization using 90% methanol for 20 mins at 4° C. Cells were washed with PBS containing 0.5% BSA (Sigma) and stained with unconjugated primary antibodies in FACS buffer overnight at 4° C. Stained cells were washed with PBS with 0.5% BSA and stained with secondary antibodies in FACS buffer for 30 mins at 4° C. Stained cells were analyzed using the LSR II Flow cytometer (BD PharMingen™). Data were analyzed using FlowJo™ software (Tree Star).

### Immunohistochemistry

The EBs were dissociated as described above and the cells were plated onto 24 well culture dishes pre-coated with Matrigel™ (25% v/v, BD PharMingen™). Cells were cultured for 2-3 days and were fixed with 4% PFA in PBS for 15 min at room temperature. Cells were permeabilized and blocked with PBS containing 5% donkey serum, 0.1% TritonX™. The following antibodies were used for staining: mouse anti-cardiac isoform of cTNT (ThermoFisher Scientific, 1:200), rabbit anti-human HEY2 (Proteintech, 1:100), mouse anti-human ANF (Abcam, 1:100), rabbit anti-human cTNT (Abcam, 1:200), or rabbit anti-human CD90 (Abcam, 1:200). For detecting unconjugated primary antibodies, the following secondary antibodies were used: donkey anti-mouse IgG-Alexa Fluor™ 488 (ThermoFisher, 1:500), donkey anti-rabbit IgG-Alexa Fluor™ 555 (ThermoFisher, 1:500), donkey anti-rabbit IgG-Alexa488 (ThermoFisher, 1:500), donkey anti-mouse IgG-Alexa Fluor™ 555 (ThermoFisher, 1:500). Detailed antibody information is described in the Key Resources Table. Cells were stained with primary antibodies in staining buffer consisting of PBS with 0.1% TritonX, and 5% donkey serum overnight at 4° C. The stained cells were washed with PBS. The cells were then stained with secondary antibodies in PBS containing 0.1% BSA for 1 h at room temperature followed by DAPI staining. For the paraffin section, tissues were fixed using 4% PFA and then embedded. After the deparaffinization and rehydration, heat-induced epitope retrieval was performed followed by immunostaining. Stained cells were analyzed using an EVOS Microscope (ThermoFisher) and Zeiss LSM700 confocal microscope (Zeiss).

### Quantitative Real-Time PCR

Total RNA from samples was isolated using RNAqueous™-micro Kit including RNase-free DNase treatment (invitrogen). RNA from dissected ventricular and atrial tissue of human fetal hearts was isolated using the TRIzol® method (ThermoFisher) and treated with DNase (Ambion®). Isolated RNA was reverse transcribed into cDNA using oligo (dT) primers and random hexamers and iScript™ Reverse Transcriptase (ThermoFisher). QRT-PCR was performed on an EP Real-Plex MasterCycler® (Eppendorf®) using QuantiFast® SYBR Green PCR kit (QIAGEN). The copy number of each gene relative to the house keeping gene TBP.

### TEM

The samples were fixed in 2.5% glutaraldehyde in PBS, rinsed and post-fixed in 1% OsO4 (Electron Microscopy Sciences) for one hour. The tissue was again rinsed with 0.1 M Sorenson's Phosphate buffer, dehydrated through an ascending ethanol series, then infiltrated with and embedded in modified Spurr's resin. From the area of interest, identified by thick sectioning, ultrathin sections (90-100 nm) were cut with a Leica UC6 ultramictrotome (Leica). Thin sections were stained with Uranyless and Lead Citrate then examined under the Hitachi HT7700 transmission electron microscope (Hitachi). Analysis were performed from 3-5 independent experiments.

### Seahorse OCR/ECAR Measurement

For the Seahorse™ XF FAO assay, a few EBs were plated onto an XFe24 cell culture microplate coated by Matrigel™ 48 hours prior to the assay. 24 hours prior to the assay, replace the culture medium with substrate-limited medium containing 0.5 mM glucose (Sigma), 1.0 mM Glutamine (Life technology), 0.5 mM Carnitine (Sigma), and 1% Fetal Bovine Serum (Wisent) in DMEM no glucose medium (ThermoFisher). 45 mins prior to the assay, wash the cells two times with FAO assay medium, add 375 μl/well FAO assay medium to the cells and incubate for 45 mins at 37 C. Load the assay cartridge with Seahorse™ XF Cell Mito Stress Test compounds (2 μM oligomycin, 5 μM FCCP, 0.5 μM rotenone/0.5 μM antimycin A). 15 mins prior to starting the assay, add 37.5 μl etomoxir (Sigma, 40 μM) or vehicle to each well. Incubate for 15 mins at 37 C in a non-CO₂ incubator and just prior to starting the assay, add 87.5 μl XF Palmitate-BSA FAO Substrate or BSA to the appropriate wells. Immediately insert the Seahorse™ XF cell Culture Microplate into the Seahorse™ XFe Analyzer and run the Seahorse™ XF Cell Mito Stress Test. After the measurement of OCR, EBs are dissociated and counted the cell number in each well. OCR was normalized per 10,000 cells. For the glycolysis assay, a few EBs were similarly prepared onto an XFe24 cell culture microplate. Prepare Glucose (Sigma) and 2-DG (Sigma) and load the assay cartridge (final concentration; glucose 10 mM, 2-DG 100 mM). Change the culture medium to glycolysis assay medium and insert the XF cell Culture Microplate into the Seahorse™ XF24 Analyzer and run the assay. After the measurement of ECAR, EBs are dissociated and counted the cell number in each well. ECAR was also normalized per 10,000 cells.

### Ca²⁺ Transient Measurement

For the Ca²⁺ transient measurement, the EBs were dissociated into single cell at day 30 and replated 2M cells onto 3.5 cm culture dish coated by Matrigel™. Culture cells in the monolayer format for a few days with culture media in each condition and load with Fluo-4 (Invitrogen, final concentration; 4 μM) for 30 minutes at 37° C. at the day of the measurement. Cells are washed by culture media and incubated by culture media for additional 30 mins. Media are switched into Tyrode buffer at 37° C. and place the plate into the system. For the imaging, the Zoom microscope body MVX10 with the objective MVPLAPO 0.63× (NA 0.15, WD 87 mm, FN 22, Olympus) for an overall FOV of 10 mm×10 mm was used with the wavelength 490-535 nm for the excitation and 532-588 nm for bandpass-filter Fluo-4. Cells are stimulated (1 Hz, pulse duration 5 ms, voltage 10V) via electrodes using PowerLab system. Ca²⁺ transients are then measured using MetaMorph software after selecting a ROI per monolayer and manually determining the start and end of each Ca²⁺ depolarisation/repolarisation through time. Data were collected from 8 to 20 samples in each condition.

### scRNA Sequencing and Analysis

The EBs were dissociated as described above and stained cells by DAPI. Live cells were then sorted using FACSAriaRITT (BD PharMingen™) at the Sickkids/UHN flow cytometry facility. After the live cell sorting, scRNA sequencing was performed and analyzed as follows.

Single-cell RNA sequencing of day 20 ventricular cardiomyocytes were first filtered to remove lowly expressed genes (defined as those found in less than 3 cells) and damaged cells with high mitochondrial genome transcript content (defined as 12 median absolute deviations above the median to account for the typically high mitochondrial content in cardiomyocytes). The data set was then normalized using the deconvolution method implemented in the scran R package (Lun et al., Genome Biology (2016) 17:75), which pools cells with similar gene expression profiles and library sizes together to normalize. We then performed principal component analysis on normalized data to reduce the number of dimensions in the data. The number of principle components to use in clustering and t-SNE was determined to be 17 by plotting the standard deviations of the first 30 components in a scree plot and selecting the point after which standard deviations are similarly minimal and thus would not contribute significantly to resolving variances between cells in downstream analyses. All cells were then iteratively clustered in Seurat 2.0 (Butler et al., Nat Biotechnol. (2018) 36, 411-420) at increasing resolutions until the number of differentially expressed genes between two neighboring clusters reached 0. We then chose the optimal clustering resolution, defined as the point where the number of clusters was maximized while the number of differentially expressed (DE) genes between neighboring clusters remained larger than 0, and annotated all clusters by examining expression of known marker genes. The chosen clustering resolution for the data set was 0.6. Finally, all cells that do not express TNNT2 were eliminated in the process of creating a cardiomyocyte-only map. Downstream differential expression analysis between clusters were done using the FindMarker function in Seurat. Results were visualized using base graphics in R

Pathway enrichment analysis: Gene Set Variation Analysis (GSVA) (Hanzelmann et al., BMC Bioinformatics (2013) 14:7) was used to identify the signaling pathways that are differentially regulated in the HEY2-high versus HEY2-low populations. Cells belonging to clusters 1 and 2 (colored as pink and yellow, respectively, in FIG. 1A) were characterized as HEY2-high, while cells belonging to cluster 4 (light green, upper left) were characterized as HEY2-low. GSVA was run on cells in these three clusters using the Enrichment Map gene sets for biological processes without electronic annotation. P-values and false discovery rates (FDR) for enriched pathways were subsequently determined using a simple linear model as implemented in the limma R package (Ritchie et al., Nucleic Acids Res. (2015) 43:e47). Pathways with FDR less than 0.05 were determined as differentially enriched between HEY2-high and HEY2-low populations. GSVA results were then visualized using EnrichmentMap in Cytoscape (Reimand et al., Nat Protoc. (2019) 14:482-517).

For the analysis of day 32 immature and mature cells, we performed the analysis as follows.

Software Tools: Scanpy (v1.4.4) (Wolf et al., Genome Biol. (2018) 19:15) and GOATOOLS (v0.9.7) (Klopfenstein et al., Sci Rep. (2018) 8:10872), and their necessary dependencies, were used for these single cell RNA sequencing analyses.

Data Preprocessing: Raw data consisted of CellRanger-processed, filtered feature matrices. Datasets were read in and concatenated using scanpy.read_10×_mtx. The combined dataset was then filtered to only contain cells with a minimum of 1500 genes and to remove genes not present in at least 10 cells using scanpy.preprocessing.filter_cells and scanpy.preprocessing.filter_cells functions. The data set was then normalized and log(x+1) transformed using the default settings in the scanpy.preprocessing.normalize_per_cell and scanpy.preprocessing.log1p functions. Principal component analysis was then performed on the transformed data using the “arpack” solver with otherwise default settings using the scanpy.tools.pca function. Finally, a neighborhood graph was computed using the scanpy.preprocessing.neighbors function with 10 neighbors specified, otherwise default settings were used.

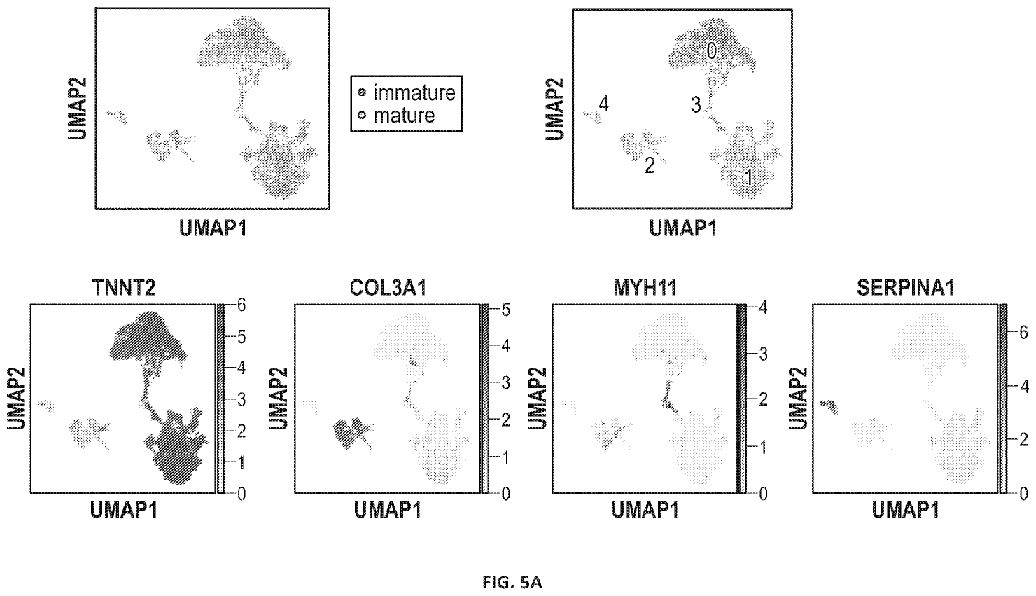

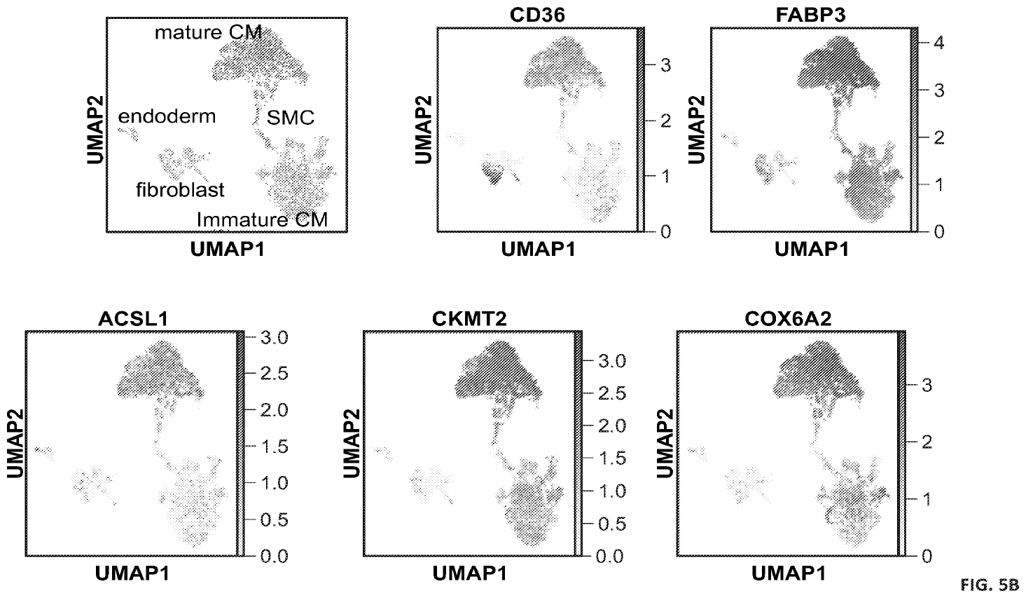

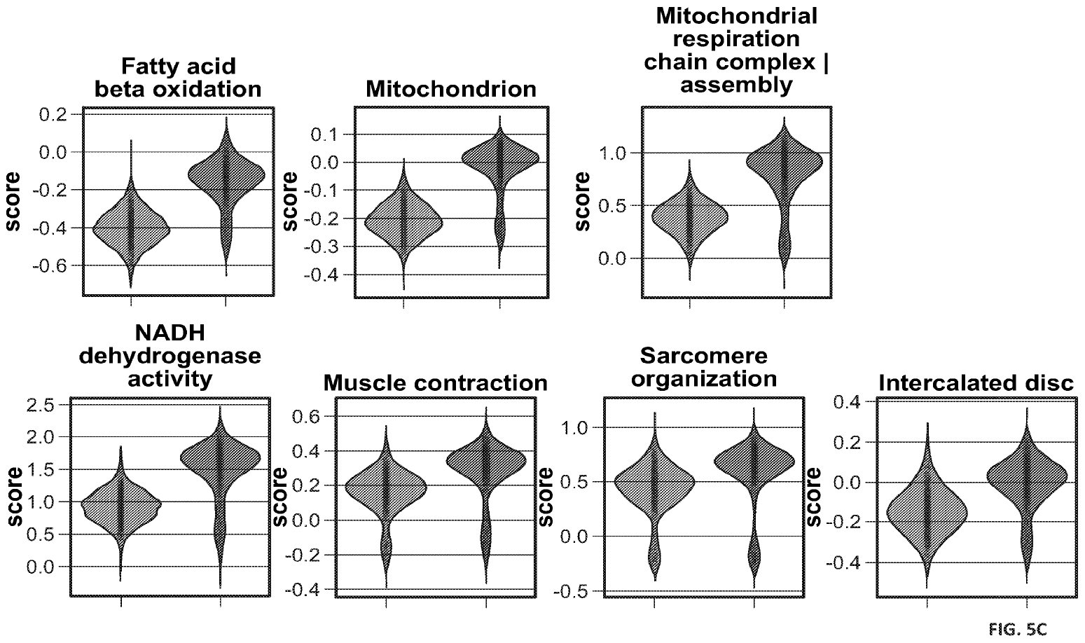

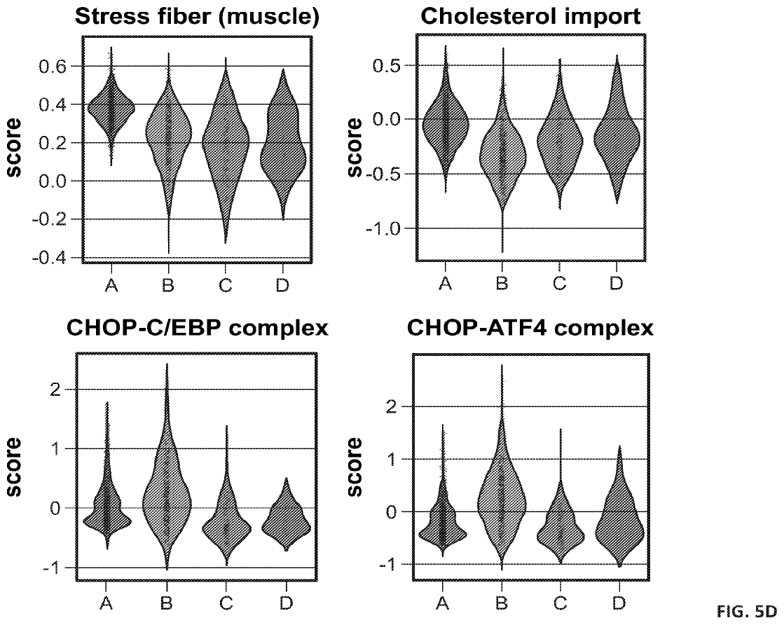

Dimensionality Reduction, Clustering and Differential Expression: μMAP coordinates were calculated using the scanpy.tools.umap function under default settings. Whole dataset clustering (Traag et al., Sci Rep. (2019) 9:5233) was performed using scanpy.tools.leiden function with a resolution of 0.2 and otherwise default settings. The dataset was subsequently subset, selecting only cluster 0 from FIG. 5A. This subset data, representing mature cardiomyocytes, was then reprocessed in isolation. PCA analysis was performed again using default settings on the subset data using only highly variable genes detected using the scanpy.preprocessing.highly_variable_genes using default settings. A neighborhood graph was then computed using the scanpy.preprocessing.neighbors function with 5 neighbors and 25 principal components specified, otherwise default settings were used. Leiden clustering was performed on the reprocessed data with a resolution of 0.2. Differentially expressed genes were discovered using the scanpy.tl.rank_genes_groups function between the previously defined Leiden groups using default settings. All genes considered for further analysis had a positive absolute z-score (underlying the computation of the p-value for each gene for each group) of greater than 1.96.

Gene Ontology Analysis: Gene Ontology Enrichment Analysis (GOEA) was performed using GOATOOLS. GO ontologies and annotations were downloaded using the goatools.base.download_go_basic_obo and goatools.base.download_ncbi_associations functions respectively. Ontologies were then loaded in using the goatools.obo_parser.GODag function. Human GO associations were then selected and stored as a list of named tuples using the call ‘taxids=[9606]’ in the goatools.anno.genetogo_reader.Gene2GoReader function. Finally the background gene set, all human protein-coding genes, were loaded in using goatools.test_data.genes_NCBI_9606_ProteinCoding.GENEID2NT function. The human ontologies, associations, and background gene set were then used for GOEA. GOEA analysis was performed on the previously discovered differentially expressed gene sets using the goatools.goea.go_enrichment_ns.GOEnrichmentStudyNS.run_study function using default settings. Enriched ontologies with a Benjamini-Hochberg-corrected p-value less than 0.05 were retained for further analysis.

Enriched gene ontology gene lists were used to generate an enrichment score using the scanpy.tl.score_genes function with default settings. Violin plots were used to highlight the difference in gene set enrichment between the previously defined Leiden clusters.

### Ki67 Flowcytometry

The EBs were dissociated as described above. The following antibodies were used for staining: mouse anti-Ki67 (DAKO, 1:100) and rabbit anti-cardiac isoform of cTNT (ThermoFisher Scientific, 1:500). The following secondary antibodies were used for detection: donkey anti-mouse IgG-APC (BD Pharmigen™, 1:500), or donkey anti-rabbit IgG-PE (Jackson ImmunoResearch, 1:500). Detailed antibody information is described in the Key Resources Table. Cells were fixed for 20 mins at 4 C with 4% PFA in PBS followed by permeabilization using 90% methanol for 20 mins at 4° C. Cells were washed with PBS containing 0.5% BSA (Sigma) and stained with unconjugated primary antibodies in FACS buffer overnight at 4° C. Stained cells were washed with PBS with 0.5% BSA and stained with secondary antibodies in FACS buffer for 30 mins at 4° C. Stained cells were analyzed using the LSR II Flow cytometer (BD PharMingen™). Data were analyzed using FlowJo™ software (Tree Star).

### Nile Red Staining by Flowcytometry

To quantify the lipid droplets, Cayman's Lipid Droplets Fluorescence Assay Kit (Cayman) was used. After the EBs were dissociated as described above, cells were fixed by Fixative Solution for 10 mins at room temperature. Cells were washed with Assay Buffer and stained with the Nile Red Staining Solution at room temperature for 15 mins. Cells were washed with Assay Buffer and analyzed with filter sets to detect FITC using the LSR II Flow cytometer (BD PharMingen™). Data were analyzed using FlowJo™ software (Tree Star).

### Contraction Force Measurement

At day 18 of differentiation, the EBs were dissociated as described above. The biowire cardiac tissues were generated and analyzed as previously described (Mastikhina et al., 2020). In brief, CMs and human cardiac fibroblasts (Lonza) were mixed in a ratio of 4:1. Cells were seeded into the microwells in fibrin gels. 2 days after the generation of the tissues, media was changed into 4 conditions followed by changing media every 3 days. At day 32, the force measurement was performed in each condition. For the force assessment in the tissues, video recordings of the rod deflection were performed under electrical stimulation (1 Hz) using a Leica EC3 camera. Image analysis was performed with ImageJ. Tissue widths were measured at the middle of the tissue and at the PDMS rods. Rod deflection for passive force was measured as the distance between the PDMS rod in the tissue's relaxed state, and the PDMS rod at non-deflected position. For force of contraction (active force), peak rod deflection under electrical stimulation was measured. To measure active force, it's total force (when a tissue is in an active contraction) minus the passive force (when it's relaxed). The measurement of tissue widths and rod deflection measurements were performed by a person blinded to the conditions. Data were collected from 7-9 independent experiments. Force calculations were performed using the following formula. f(x, y)=1.55 x+0.00256 x2+0.002156 xy. The function f represents force (N), while xis the PDMS rod deflection from its non-deflected position origin, and y is the tissue width at the midspan of the PDMS rod. After the force measurement, tissues were fixed for immunohistochemistry or TEM analysis.

### Annexin V Apoptosis Assay

After the culture in the pathological condition for 6 days, the EBs were dissociated as described above. To detect the apoptosis following the pathological stimuli, TACS® Annexin V assay (TREVIGEN) was performed by flowcytometry. Cells were washed with PBS and stained with Annexin V-FITC for 15 mins at room temperature. Add Binding Buffer to samples and process by flowcytometry. Stained cells were analyzed using the LSR II Flow cytometer (BD). Data were analyzed using FlowJo™ software (Tree Star).

### Cell Transplantation into Rat MI Models

The EBs were dissociated as described above and cryopreserved using Cryostor® (STEMCELL Technologies) prior to the cell transplantation.

Rat myocardial infarction model: A permanent coronary ligation technique was used to generate myocardial infarction in athymic nude rat hearts. All rats were intubated and positive pressure ventilation was maintained with a Harvard ventilator under the anesthesia with inhalational isoflurane 2-3%. The rat heart was exposed through a left anterolateral thoracotomy incision. A 7-0 suture was used to permanently ligate the left anterior descending artery.

Thoracotomy and cell transplantation: The nude rats undergo cell transplantation 3-4 days after the induction of myocardial infarction. Rats were anesthetized and ventilated as described above. The heart was exposed and 10×10⁶ cells were injected with 75 μl Matrigel™ (100%, BD PharMingen™) using a 30G needle into the infarcted region of the heart.

Sacrifice and analysis: 2 weeks following the transplantation, rats were sacrificed and their hearts were harvested. The hearts were fixed by 10% formaldehyde and were processed to immunostaining. The hearts were sectioned horizontally into 12 levels to cover all the LV area. The following antibodies were used for imunostaining: mouse anti-cardiac isoform of cTNT (ThermoFisher Scientific, 1:200), rabbit anti-human cTNT (Abcam, 1:200), rabbit anti-GFP (ROCKLAND, 1:200), mouse anti-Ki67 (DAKO, 1:100), rabbit anti-CX43 (Abcam, 1:800). For detecting unconjugated primary antibodies, the following secondary antibodies were used: donkey anti-mouse IgG-Alexa™ 488 (ThermoFisher, 1:500), donkey anti-rabbit IgG-Alexa488 (ThermoFisher, 1:500), donkey anti-mouse IgG-Alexa555 (ThermoFisher, 1:500), or donkey anti-rabbit IgG-Alexan Fluor™ 555 (ThermoFisher, 1:500). Sarcomere length was measured in cTNT(+) grafted CMs randomly picked up from 5-10 areas in grafted CMs. CX43 expression was measured by counting the number of CX43(+) staining in one field of view (×40 magnification) in cTNT(+) grafted CMs randomly picked up from 5-10 areas in grafted CMs. Graft size was measured by calculating the ratio GFP(+) graft area divided by all LV area. All those imaging were taken by Zeiss LSM700 confocal microscope and analyzed by ImageJ.

### Quantification and Statistical Analysis

All data are represented as mean±standard error of mean (SEM). Indicated sample sizes (n) represent biological replicates including independent cell culture replicates and individual tissue samples. For single cell data, samples size represents the number of cells analyzed from at least three independent experiments. No statistical method was used to predetermine the samples size. Statistical significance was determined by using Student's t test (unpaired, two-tailed) or one-way ANOVA with Tukey's multiple comparisons in GraphPad Prism 6 software. All statistical parameters are reported in the respective figures and figure legends.

### Example 1: Cardiac Differentiation Protocol 1

In an exemplary, nonlimiting protocol described in Lee, supra, the human MSC-iPSC1 line (karyotype: 46, XY; from Harvard Medical School), the human HES2 ESC line (karyotype: 46, XX; from WiCell), or the human HES3 ESC line (karyotype: 46, XX; from Monash University) is used. The hPSC cells are maintained on irradiated mouse embryonic fibroblasts in hPSC culture media consisting of DMEM/F12 (Cellgro) supplemented with penicillin/streptomycin (1%, ThermoFisher), L-glutamine (2 mM, ThermoFisher), nonessential amino acids (1×, ThermoFisher), β-Mercaptoethanol (55 mM, ThermoFisher), and KnockOut™ serum replacement (20%, ThermoFisher) as described previously (Kennedy et al., supra). For cardiac differentiation, the hPSC populations at 80%-90% confluence are dissociated into single cells (TrypLE™, ThermoFisher) and re-aggregated to form embryoid bodies in StemPro™-34 media (ThermoFisher) containing penicillin/streptomycin (1%, ThermoFisher), L-glutamine (2 mM, ThermoFisher), transferrin (150 mg/ml, ROCHE), ascorbic acid (50 mg/ml, Sigma), and monothioglycerol (50 mg/ml, Sigma), ROCK inhibitor Y-27632 (10 mM, TOCRIS) and rhBMP4 (1 ng/ml, R&D) for 18 hours on an orbital shaker.

At day 1, the embryoid bodies (EBs) are transferred to mesoderm induction media consisting of StemPro™-34 with above supplements (-ROCK inhibitor Y-27632) and rhBMP4, rhActivinA (R&D) and rhbFGF (5 ng/ml, R&D).

At day 3, the EBs are harvested, washed with IMDM and transferred to cardiac mesoderm specification media consisting of StemPro™-34, the Wnt inhibitor IWP2 (1 mM, TOCRIS) and rhVEGF (10 ng/ml, R&D).

At day 5, the EBs are transferred to StemPro™-34 with rhVEGF (5 ng/ml) for another 7 days and then to StemPro™-34 media without additional cytokines for further 8 days.

At day 20, HES2-derived cardiomyocytes are analyzed and isolated based on the expression of SIRPA and a lack of CD90. Cardiomyocytes generated from non-transgenic hPSC lines are analyzed and isolated as SIRPA⁺CD90⁻ populations.

Media are changed every 3 days. Cultures are incubated in a low oxygen environment (5% CO₂, 5% O₂, 90% N₂) for first 12 days and a normoxic environment (5% CO₂) for the following 8 days in total of 20 days. The EBs are cultured in ultra-low attachment 6-well dishes (Corning) throughout the differentiation for maintaining suspension cultures.

### Example 2: Cardiac Differentiation Protocol 2

In an exemplary, nonlimiting protocol based on the protocol described in Lian et al., supra, cardiomyocytes (including ventricular progenitor cells) may be generated from human PSCs via cardiac induction using CHIR as follows.

At day −1, 6E6 hPSCs are plated and cultured on Vitronectin-coated six-well plates in E8 medium and allowed to attach to the plates overnight.

At day 0, cell culture medium is prepared by adding CHIR-99201 (“CHIR”; Tocris 4423/10) to basal cardiomyocyte (CM) medium (RPMI (with L-Glutamine)/B-27 without insulin, plus 213 μg/ml L-ascorbic acid 2-phosphate (Sigma)) to reach a CHIR concentration of 2, 4, 6, 8, 10, or 12 μM. The old medium in the plates is replaced with 4 ml per well of CHIR-supplemented basal CM medium. Optimization of CHIR concentration may be desirable (e.g., a range of 2-12 μM CHIR may be tested).

At day 1, the culture medium is removed by aspiration. The wells are washed once with DMEM to remove debris. Then room-temperature RPMI/B-27/without insulin medium is added at a volume of 4 ml per well. The plates are incubated at 37° C., 5% CO₂.

At days 2 to 3, the culture medium is removed by aspiration. The wells are washed once with DMEM to remove debris. Then IWR1 is added to 4 ml of fresh RPMI/B-27/without insulin medium, to reach a final IWR1 concentration of 2.5 μM.

At day 5, the culture medium is replaced with room-temperature RPMI/B-27/without insulin medium at a volume of 4 ml per well. The plates are incubated at 37° C., 5% CO₂. At this point, the cell culture comprises cardiac progenitor cells.

From day 7 and on, the culture medium is replaced with room-temperature RPMI/B-27 medium at a volume of 4 ml per well. The plates are incubated at 37° C., 5% CO₂.

Cardiomyocytes are counted by flow cytometry (cTNT/NKX2-5). Robust spontaneous contraction should occur by day 12. The cells can be maintained with this spontaneously beating phenotype for more than 6 months.

### Example 3: Cardiac Differentiation Protocol 3

The cell culture method used in the Working Examples below is described as follows. For cardiac differentiation, we used a modified version of the EB-based protocol (Kattman et al., supra). In this version, hPSC populations at 80-90% confluence were dissociated into single cells and re-aggregated to form EBs in StemPro™-34 media (ThermoFisher) containing penicillin/streptomycin (1%, ThermoFisher), L-glutamine (2 mM, ThermoFisher), transferrin (150 mg/ml, ROCHE), ascorbic acid (50 mg/ml, Sigma), and monothioglycerol (50 mg/ml, Sigma), ROCK inhibitor Y-27632 (10 mM, TOCRIS), and rhBMP4 (1 ng/ml, R&D) for 18 h on an orbital shaker.

At day 1, the EBs were transferred to mesoderm induction media consisting of StemPro™-34 with the above supplements (-ROCK inhibitor Y-27632) and rhBMP4, rhActivinA (R&D) and rhbFGF (5 ng/ml, R&D) at the indicated concentrations.

At day 3, the EBs were harvested, washed with IMDM and transferred to cardiac mesoderm specification media consisting of StemPro™-34, the Wnt inhibitor IWP2 (1 mM, TOCRIS) and rhVEGF (10 ng/mL, R&D).

At day 5, the EBs were transferred to StemPro™-34 with rhVEGF (5 ng/ml) for another 5 days and then to DMEM high glucose (Life Technologies) with 0.5 mg/mL human serum albumin (Sigma), ascorbic acid, transferrin, insulin (10 ng/ml, Sigma), CHIR (1 μM, Bio-Techne), and IGF2 (25 ng/ml, Bio-Techne) from day 10 to day 16.

At day 16, cells were transferred to DMEM high glucose with 1%-B27 minus insulin supplement (Life Technologies), 0.5 mg/mL human serum albumin (Sigma), ascorbic acid, transferrin, and XAV (4 μM, Bio-Techne).

From day 18, cells were transferred to DMEM glucose 2 g/L (Life Technologies) with 1%-B27 minus insulin supplement, human serum albumin, ascorbic acid, transferrin, palmitic acid (200 μM, Sigma), GW7647 (1 μM, Sigma), Dexamethasone (100 ng/ml, Bio-Shop), and T3 (4 nM) for 2 weeks. Cultures were incubated in a low oxygen environment (5% CO₂, 5% O₂, 90% N₂) for first 12 days and a normoxic environment (5% CO₂) for the following 20 days in total of 32 days. The EBs were cultured in 6 cm petri dish from day 0 to day 10, and then in polyheme-coated 10 cm dishes (Corning) with 70 rpm rotation.

### Example 4: Cardiac Differentiation Protocol 4

For ventricular differentiation, hPSC populations (HES2) were dissociated into single cells (TrypLE™, ThermoFisher) and re-aggregated to form EBs in StemPro™-34 media (ThermoFisher) containing penicillin/streptomycin (1%, ThermoFisher), L-glutamine (2 mM, ThermoFisher), transferrin (150 mg/ml, ROCHE), ascorbic acid (50 mg/ml, Sigma), and monothioglycerol (50 mg/ml, Sigma), ROCK inhibitor Y-27632 (10 mM, TOCRIS) and rhBMP4 (1 ng/ml, R&D) for 24 h on an orbital shaker (70 rpm).

At day 1, the EBs were transferred to mesoderm induction media consisting of StemPro™-34 with above supplements (-ROCK inhibitor Y-27632) and rhBMP4 (8 ng/ml), rhActivin A (12 ng/ml, R&D) and rhbFGF (5 ng/ml, R&D).

At day 3, the EBs were harvested, dissociated into single cells (TrypLE™), and re-aggregated in cardiac mesoderm specification media consisting of StemPro™-34, the Wnt inhibitor IWP2 (1 mM, TOCRIS) and rhVEGF (10 ng/mL, R&D).

At day 5, the EBs were transferred to StemPro™-34 with rhVEGF (5 ng/ml) for another 5 days and then to DMEM high glucose (4.5 g/l, ThermoFisher) media with compact factors [Wnt signaling agonist step] (CHIR (1 μM, TOCRIS), IGF2 (25 ng/ml, R&D)) at day 10 for another 6 days.

From day 16 to day 18, the EBs were transferred to DMEM high glucose media with XAV (4 μM, TOCRIS) and then transferred to maturation media (DMEM containing 2 g/l glucose with Palmitic acid (200 μM, Sigma), Dexamethazone (100 ng/ml, Bioshop), T3 hormone (4 nM, Sigma) and GW7647 (PPARA agonist, 1 μM, Sigma)) for the following 9 days.

Finally, the EBs were cultured in DMEM containing 2.0 g/l glucose with Palmitate (200 μM) alone for the following 5 days in total 32 days.

Cultures were incubated in a low oxygen environment (5% CO₂, 5% O₂, 90% N₂) for first 12 days and a normoxic environment (5% CO₂, 20% O₂) for the following 20 days. From day 10 to day 32, the EBs were cultured in polyheme-coated low binding 10 cm culture dishes on an orbital shaker (70 rpm).

For atrial differentiation, we used the different concentration of rhBMP4 (3 ng/ml) and rhActivin A (1 ng/ml) from day 1 to day 3, followed by Retinoic Acid (0.5 μM, Sigma) from day 3 to day 5 as previously described (Lee, supra).

For the atrial maturation process, we used the same maturation media as the ventricular maturation from day 18 to day 32.

### Example 5: Specification and Proliferation of Ventricular Cardiomyocyte Cells of the Compact Lineage

This Example describes methods for generating ventricular cardiomyocytes of the compact lineage. Beyond their positions and functions within the developing heart, compact and trabecular ventricular cardiomyocytes can be distinguished based on differential expression of specific markers. For example, HEY2 and MYCN are preferentially expressed in the compact cardiomyocytes, whereas ANF and BMP10 are found at higher levels in the trabecular cells. To determine the type of ventricular cardiomyocyte produced with the protocol described in Lee et al., Cell Stem Cell (2017) 21(2):179-94, we carried out single cell RNA sequence (scRNAseq) analyses of day 20 cell populations generated under these conditions. T-SNE plots of clustered scRNAseq data identified 9 distinct groups within the cTNT(+) population (FIG. 1A). While the majority of cells expressed HEY2, indicative of a compact fate, ANF⁺ trabecular cells were also detected (cluster 4), indicating that the population represented a mixture of both cell types (FIG. 1B). More detailed analyses of clusters 1 and 2 that expressed high levels of HEY2 and cluster 4 that showed high levels of ANF confirmed this lineage assignment. HIF1A and CCND2, genes associated with compact myocardium, were expressed at higher levels in clusters 1 and 2 than in cluster 4 whereas SCN5A and IRX3 known to be preferentially expressed in trabecular myocardium in vivo showed the opposite pattern. The general ventricular markers, cTNT and MYL2, were detected in all 3 clusters.

To gain insights into signaling pathways that may control the development of these cardiomyocyte subpopulations, we carried out pathway analyses of clusters 1, 2, and 4 to identify those pathways that were upregulated in the HEY2⁺ cells compared to the ANF⁺ cells (FIG. 1C). These analyses identified a number of differentially regulated pathways: the netrin 1 signaling pathway, the canonical Wnt signaling pathway, the IL-35-mediated signaling pathway, the JAK-STAT cascade, the Notch signaling pathway, the non-canonical Wnt signaling pathway, the toll-like receptor 2 signaling pathway, downstream TCR signaling pathway, the RET signaling pathway, and the pathway for regulation of TNFR1 signaling. Among these, the largest differentials being observed were associated with the Netrin 1 and Wnt signaling signaling pathways. Given this, we focused our effort on investigating the effects of manipulating the Wnt/β-catenin signaling on the generation and proliferation of hPSC-derived compact cardiomyocytes. Additionally, it has been reported that IGF2 secretion from epicardium promotes compact layer proliferation in the developing heart. Based on these observations, we also investigated the role of IGF2 signaling in the generation of hPSC-derived compact cardiomyocytes.

For these studies, we used our previously published protocol to generate cardiomyocytes (Lee, supra). Using Ki67 as a measure of proliferation, we found that the highest proportion of Ki67+cTNT+ cardiomyocytes was detected between days 12 and 14 of culture in the absence of any added cytokines. Using this timeframe as an indication of the proliferative stage of ventricular development, between days 10 and 16 of differentiation, we added the small molecule GSK-3 inhibitor CHIR-99021 (“CHIR”; a WNT pathway agonist), or IGF2, or both, to the day 10 cell population (FIG. 1D). As a control, we added neuregulin 1 (NRG) to the cultures as it promotes the development of the trabecular myocardium in the developing heart (see, e.g., Del Monte-Nieto et al., Nature (2018) 557(7705):439-45; and Odiete et al., Circ Res. (2012) 111(10):1376-85). The cells were cultured with these factors for 6 days. The cells were then harvested, counted, and analyzed for expression of compact and trabecular genes by RT-qPCR. The addition of either CHIR (1 μM) or IGF2 (25 ng/ml) led to a significant increase in the proportion of of Ki67⁻cTNT⁺cardiomyocytes detected at day 16 (FIG. 1E). The addition of both factors promoted the greatest expansion in cell number, resulting in a 2-fold increase. Activation of the Wnt pathway led to a significant increase in the expression of MYCN and a decrease in the expression of the trabecular markers ANF and BMP10 (FIG. 1F). It had little effect on HEY2, which was already expressed at reasonable levels within the population. IGF2 signaling had no effect on the expression of these genes. Addition of NRG reduced the expression of HEY2 and increased the expression of ANF, indicative of specification of a trabecular fate.

Expression of markers indicative of ventricular cardiomyocytes (e.g., cTNT and IRX4) were not significantly impacted by the addition of any of these factors. The addition of CHIR increased the expression of the glucose transporter GLUT1 and reduced the expression of the fatty acid transporter, CD36, suggesting that this proliferative stage is largely dependent on glycolysis.

Immunostaining analyses confirmed our scRNAseq findings and showed that the untreated day 16 ventricular population (negative) was made up of HEY+ compact cardiomyocytes and about 20% of ANF+ trabecular cells (FIGS. 1G and 1H). ANF+ cells were not detected in the CHIR/IGF2-treated population, indicating that these manipulations promoted the development of a highly enriched HEY2+ compact population (compact). By contrast, treatment with NRG efficiently promoted a trabecular fate as demonstrated by the presence of ANF+ cells and the lack of HEY+ compact cells (trabecular).

Together, these data show that it is possible to efficiently specify the compact fate in developing cardiomyocytes through manipulation of the Wnt and IGF2 pathways between days 10 and 16 of differentiation.

### Example 6: Identification of Factors that Promote the Metabolic Switch from Glycolysis to Fatty Acid Oxidation in Compact Cardiomyocytes

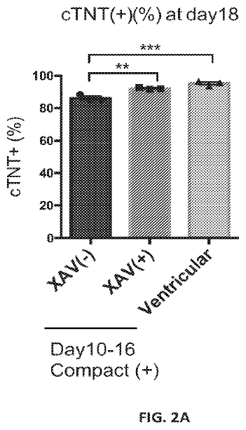

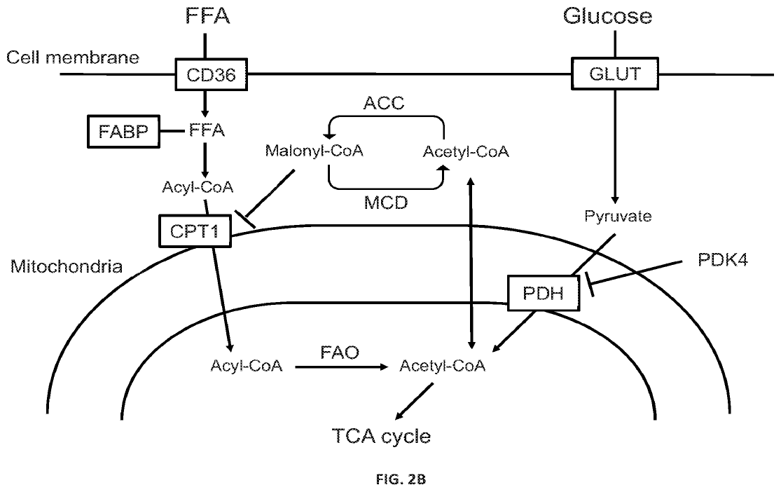

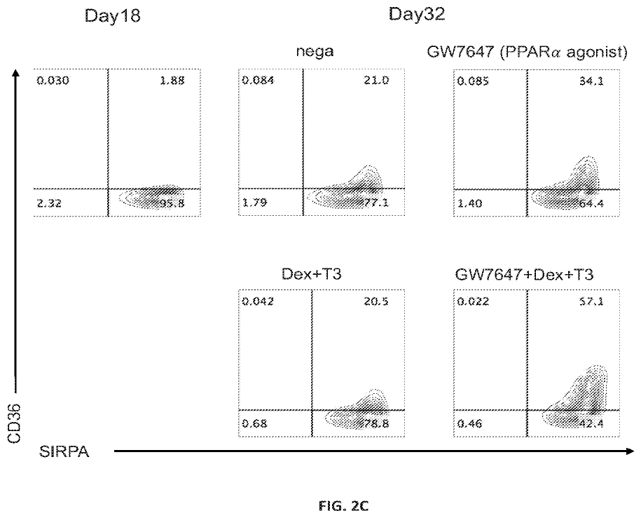

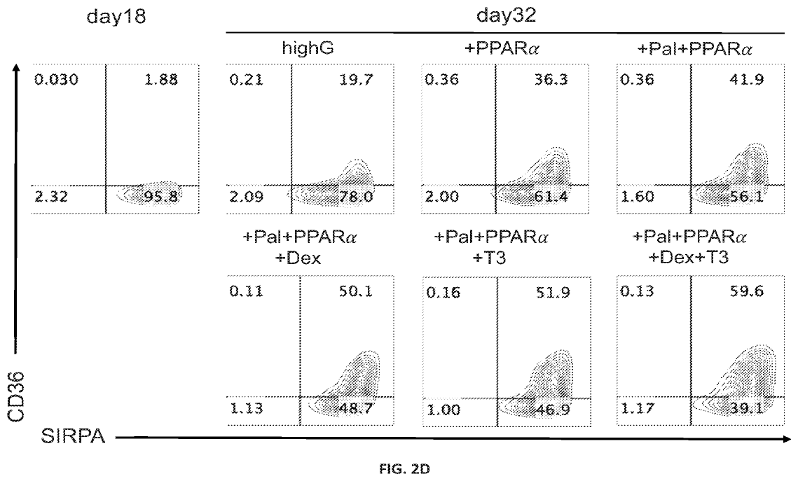

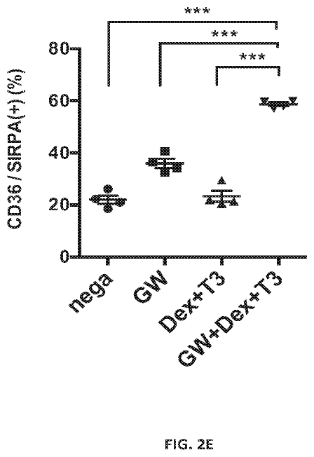

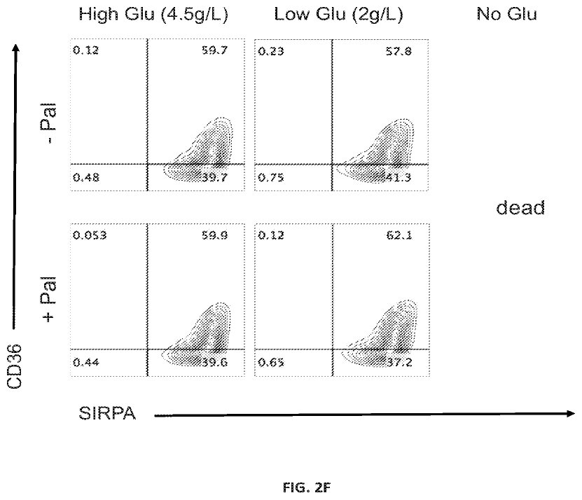

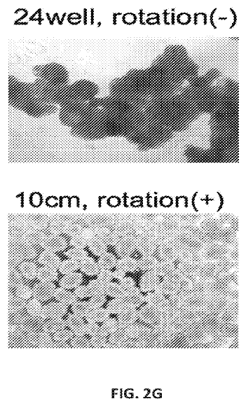

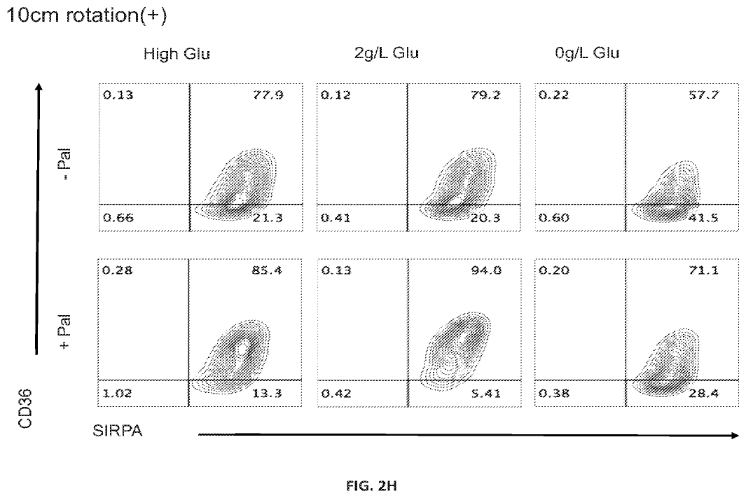

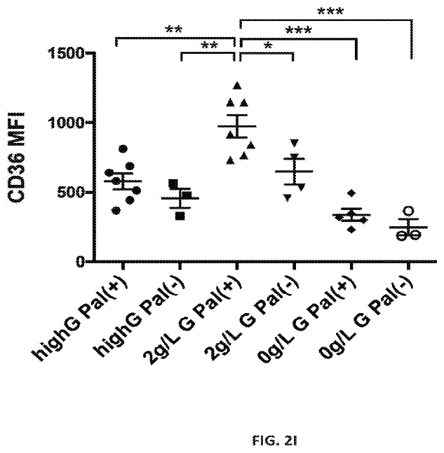

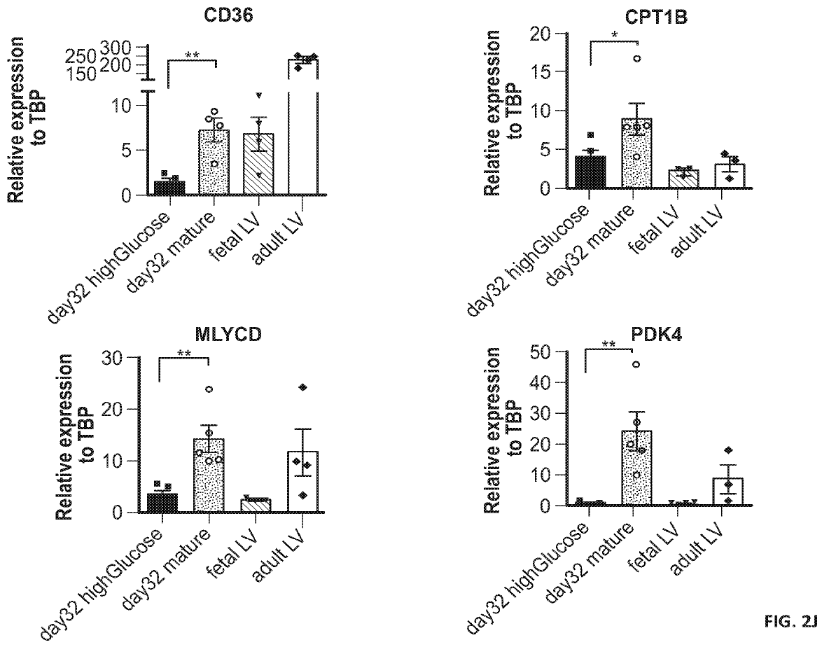

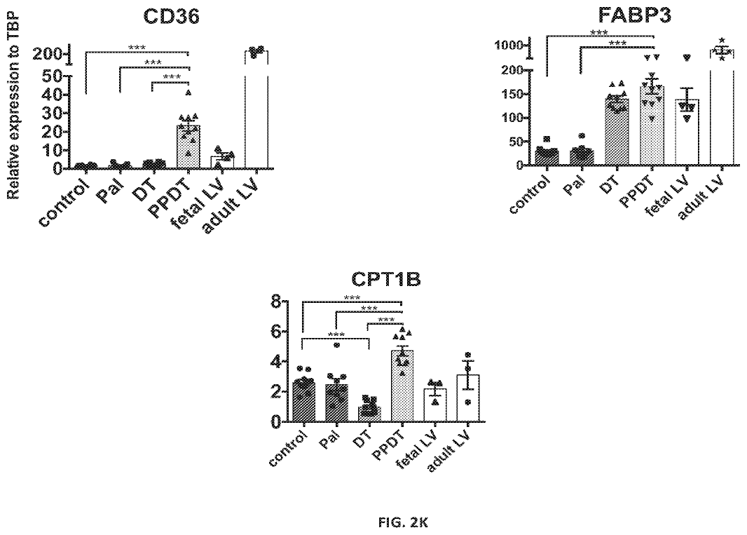

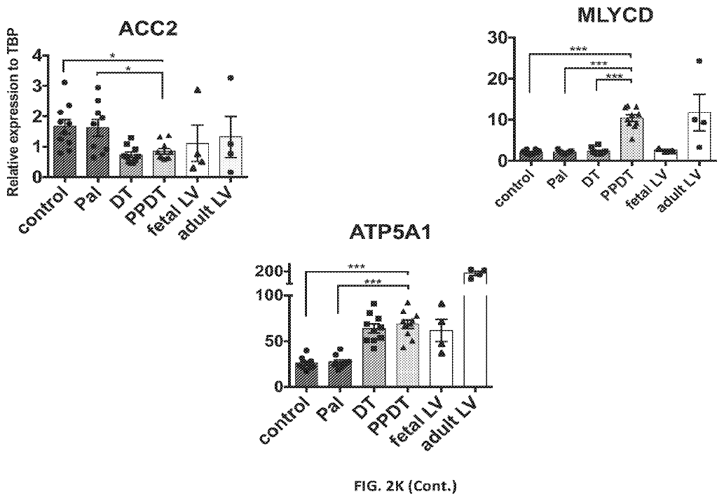

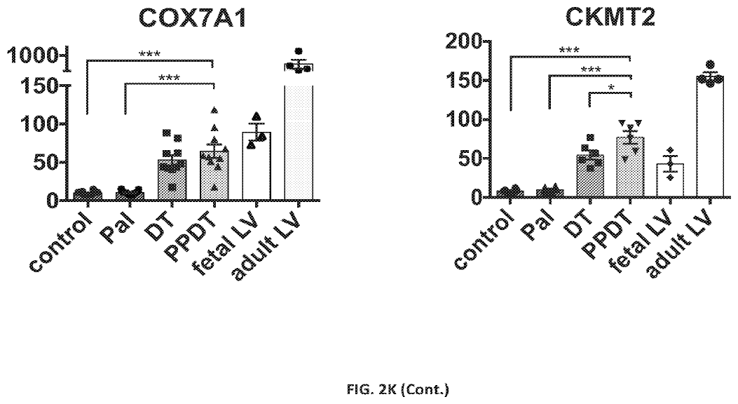

Following the proliferative stage, compact cardiomyocytes undergo a series of maturation steps, one of the most notable being a shift in energy metabolism from glycolysis to fatty acid oxidation (FAO). Cardiomyocyte maturation is associated with a reduction in the proliferative activity of the cells. To mimic this in our cultures and to promote the exit from cell cycle, we treated the developing cardiomyocyte population with the Wnt signaling antagonist Xav-939 (XAV) for 2 days to inhibit the Wnt-induced proliferation. CHIR treatment expanded both the cardiomyocyte and non-cardiomyocyte population as demonstrated by the reduction in the proportion of cTNT⁺ cells in the population (FIG. 2A; XAV(−) vs. ventricular). Addition of XAV for 2 days led to a reduction in the proportion of the non-cardiomyocytes (FIG. 2A), resulting in an enriched cardiomyocyte population relative to populations not treated with XAV. The addition of XAV following the CHIR/IGF2-induced proliferative stage also significantly reduced the proportion of Ki67⁺ (a proliferation marker) cell in the population. The differential effect of XAV on these cells suggests that the non-cardiomyocytes are proliferating faster than the cardiomyocytes.

To evaluate the capacity of the day 18 compact cardiomyocytes to undergo FAO, we analyzed them for expression of the fatty acid transporter CD36, which is essential for transporting fatty acid into cells (FIG. 2B). Given that most mature cardiomyocytes in the human heart express CD36, flow cytometric analyses of this transporter would provide rapid quantitative measure of the maturation status of these cells. Flow cytometric analyses revealed that CD36 was not expressed in the day 18 compact population (FIG. 2C and 2D). This observation indicates that these cells were metabolically immature. Following an additional 14 days of culture, a small subset of the population expressed low levels of CD36, suggesting that the cells are undergoing the initial stages of the metabolic transition to FAO in the absence of additional manipulations.

To promote the metabolic switch in cardiomyocytes, we first focused on PPAR-related signaling, a pathway known to regulate FAO and mitochondrial function. Specifically, we evaluated the effects of PPAR-related signaling, the response to steroid (dexamethasone) and thyroid (T3) hormones and the response to FA (palmitate), all of which have been shown to regulate FAO and mitochondrial function (see, e.g., Finck and Kelly, J Mol Cell Cardiol. (2002) 34(10):1249-57; Fan and Evans, Curr Opin Cell Biol. (2015) 33:49-54; Lopaschuk and Jaswal, J Cardiovasc Pharmacol. (2010) 56(2):130-40; and Finck et al., J Clin Invest. (2002) 109(1):121-30); Hirose et al., Science (2019) 364:184-8; and Rog-Zielinska et al., Cell Death Differ. (2015) 22:1106-16). Palmitate is a common dietary fatty acid that can be incorporated into multiple fatty acid synthesis and oxidation pathways.

Addition of the PPARa agonist (GW7647, 1 μM) to day 18 CMs led to a significant increase in the size of CD36+ population detected at day 32 of culture (FIGS. 2C and 2D). The addition of the PPARα agonist (GW7647) to day 18 cardiomyocytes cultured in high glucose (4.5 g/L) containing media led to an increase in the size of CD36+ population detected at day 32 of culture. In addition to PPARα signaling, we also investigated the effects of a number of other pathways that had been reported to impact mitochondrial function and FAO, including growth hormone, estrogen-related-receptor signaling, insulin like growth factors (IGFs), steroid hormone, and thyroid hormone (see, e.g., Fan and Evans, Curr Opin Cell Biol. (2015) 33:49-54; Montessuit et al., Pflugers Arch. (2006) 452(4):380-6; Rog-Zielinska et al., Cell Death Differ. (2015) 22(7):1106-16; Pucci et al., Int J Obes Relat Metab Disord. (2000) 24 Suppl 2:S109-12; and Moller and Jorgensen, Endocr Rev. (2009) 30(2):152-77). Of these, dexamethasone (Dex) and thyroid hormone (T3) had the largest effect on CD36 expression. This effect, however, was only observed in the presence of PPARα signaling.

Neither dexamethasone (Dex) nor thyroid hormone (T3) added together with PPARa and palmitate impacted the size of the CD36⁺ population. However, the addition of both hormones with the PPARα agonist and palmitate did promote the development of a significantly larger CD36⁺ subpopulation, which represented approximately 50% of the total day 32 SIRPA⁺ cardiomyocyte population (FIG. 2D). Hence, in the presence of GW7647, Dex and T3, more than half of the day 18 cardiomyocyte population expressed CD36 (FIGS. 2C-E).

In the presence of this combination of signaling factors, we next manipulated glucose concentration, culture format, and lipid formulations to optimize the generation of CD36⁺ cells. Specifically, we investigated the effects of reducing or eliminating glucose from the media in an effort to further increase the generation of CD36⁺ cells and promote the use of fatty acids (FAs) such as palmitate. In our standard 24-well culture dish, the elimination of glucose from the media (to promote the use of fatty acids) resulted in embryoid body (EB) clumping and massive cell cardiomyocyte death (FIG. 2F). To overcome this problem, we switched to larger culture dishes (10 cm). In this format, we were able to rotate the cultures, which reduced clumping and maintained the cells in small, uniformly sized aggregates (FIG. 2G). Under these culture conditions, the cells survived well in the presence of both high (4.5 g/L) and low (2 g/L) glucose concentrations (FIG. 2H). Cardiomyocytes also survived in the absence of glucose, although significant cell death (30%) was still observed.

We next investigated the consequences of adding palmitate as a source of fatty acids to the cultures. Palmitate was chosen as it is a common dietary fatty acid and can be incorporated into multiple fatty acid synthesis and oxidation pathways. As shown in FIG. 2I, the addition of palmitate to the rotation cultures led to a significant increase in the proportion of cardiomyocytes that expressed high levels of CD36⁺ (MFI 1000) in the population cultured in media containing 2 g/L glucose. In addition to improved cell survival, the modified culture format supported the efficient induction of CD36⁺ cells (>90%) following treatment with the combination of the PPARα agonist, palmitate, Dex, and T3 (PPDT) in the presence of low glucose. Greater than 90% of the cells cultured under these conditions were CD36⁺. These findings provided the basis for the protocol shown in FIG. 4D.

RT-qPCR analyses of the cell populations revealed that as compared control cells, the palmitate-treated cells expressed significantly higher levels of CD36 as well as CPT1B (a transporter of fatty acids into the mitochondria), MLYCD (an enzyme that converts malonyl COA to acetyl COA), and PDK4 (inhibitor of glycolysis through inhibiting pyruvate dehydrogenase) (FIG. 2J).

With these optimized conditions, we next compared the efficiency of CD36 induction with PPDT to factors previously reported to induce cardiomyocyte maturation, including fatty acids in low glucose media (palmitate) and the combination of Dex and T3 in high glucose media (DT) (FIG. 2K) (Mills et al., Proc Natiol Acad Sci. (2017) 114:E8372-81; Parikh et al., Circ Res (2017) 121:1323-30; Yang et al., Stem Cell Reports (2019) 13:657-68). Under these conditions, Palmitate alone was not effective in promoting the development of CD36⁺ cells. The combination of Dex and T3 did induce a sizeable CD36⁺ population; however, it was not as effective as the combination of PPDT which consistently generated cardiomyocyte populations of greater than 90% CD36+ cells.

Upon entry into the cell, FAs are shuttled through the cytoplasm to the mitochondria by FABPs and then into the mitochondria by the transporter CPT1(FIG. 2B). CPT1 mediated transport is inhibited by malonyl CoA, the levels of which are regulated by MLYCD, an enzyme that converts malonyl CoA to acetyl CoA, and ACC2, that converts acetyl CoA to malonyl CoA. The RT-qPCR analyses revealed that treatment with PPDT led to significant increases in the expression levels of the above components of the FAO pathways, including CD36, FABP3, CPT1B, MLYCD, over those detected in untreated populations. The level of ACC2 by contrast was significantly downregulated (FIG. 2K). With the increased expression of genes associated with FAO, we observed an upregulation of expression of genes that encode components of mitochondrial function and the electron transport chain, including ATP5A1, COX7A1, and CKMT2. Treatment with palmitate alone had no effect on the expression patterns of any of these genes, whereas addition of DT did induce the upregulation of FABP3, CKMT2, COX7A1 and ATP5A, but not CPT1B or MLYCD.

Collectively, these results show that the combination of the PPARα agonist, dexamethasone, thyroid hormone T3, and palmitate in low glucose media induces hPSC-derived compact ventricular cardiomyocytes to undergo metabolic changes that were indicative of a switch from glycolosis to FAO. In the following Examples, these cardiomyocytes will be referred to as the “mature” cells. Day 32 cardiomyocytes cultured in high glucose media in the absence of any of these factors will be used as control or referred to as “high glucose” cells.

### Example 7: Metabolic Analyses of Mature Compact Cardiomyocytes Revealed a High Respiration Rate and a High Level of Lipid Storage within the Cells

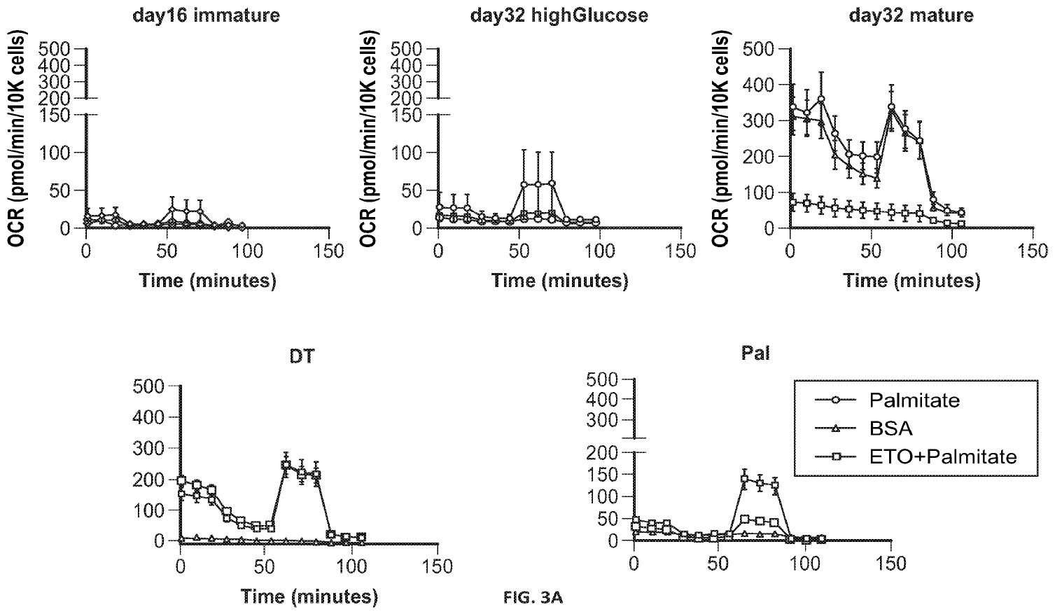

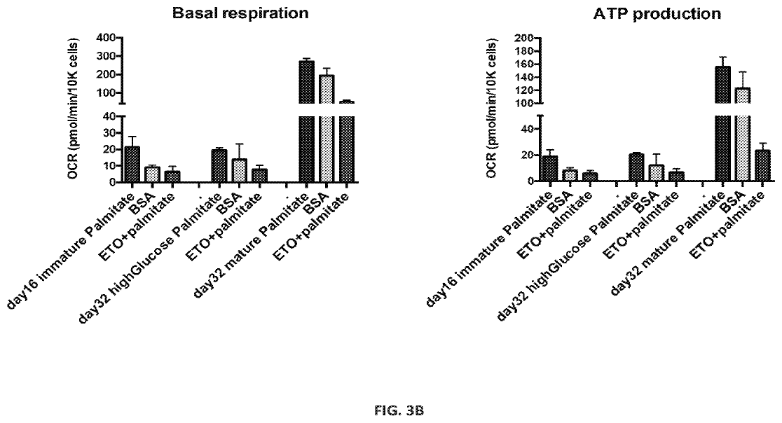

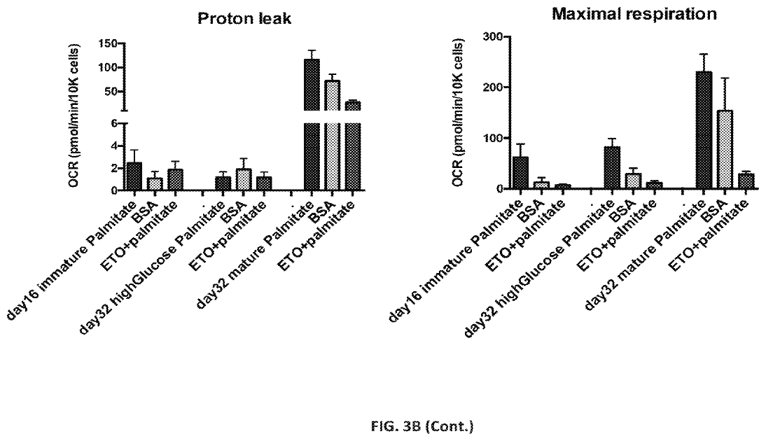

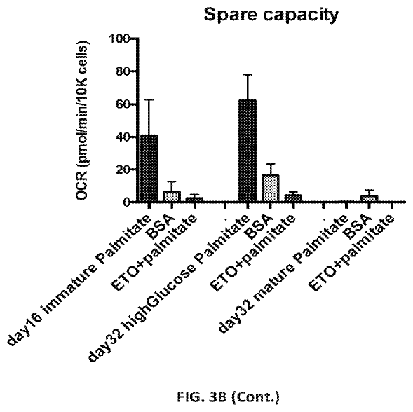

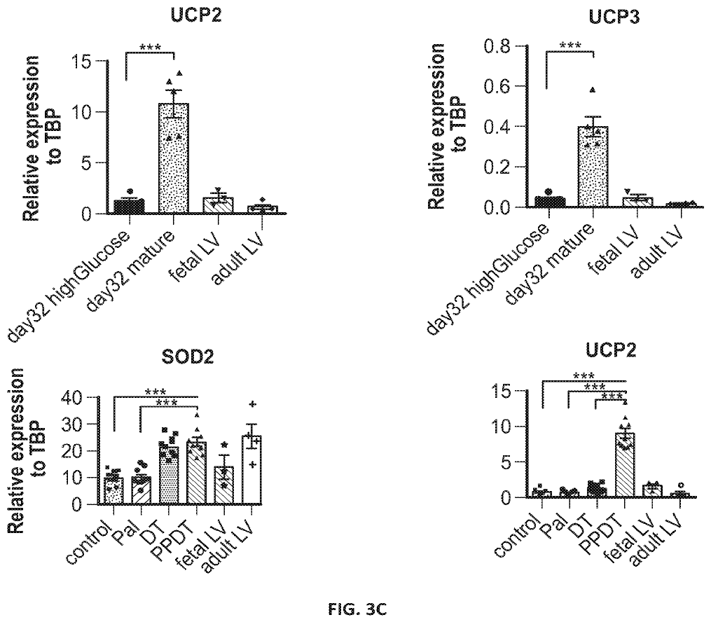

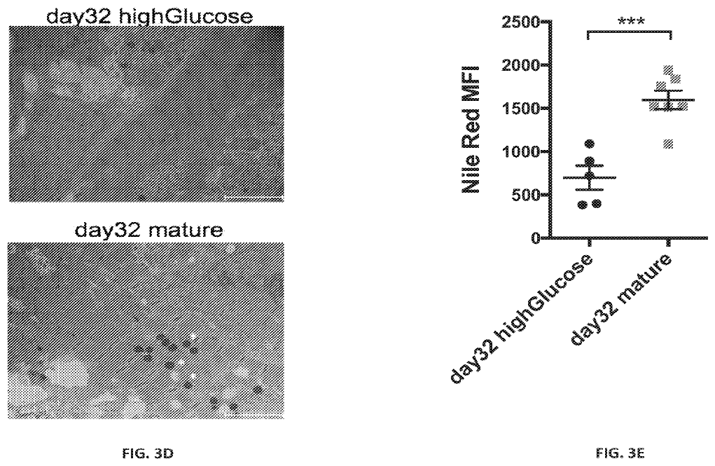

To functionally assess the capacity of the treated and control cardiomyocytes to import and metabolize exogenous fatty acids, we used the Seahorse™ XF assay to measure oxygen consumption rate (OCR) in the presence of palmitate or BSA (control). Quantification of the OCR parameters revealed that the mature (PPDT-treated) cells demonstrate higher basal metabolism, ATP production, proton leak, and maximal respiration capacity, than untreated cells (control population) or those treated with Pal or DT (FIGS. 3A and 3B). Addition of etoximir (ETO, red bar), an inhibitor of FAO (inhibitor of CPT1), blocked mitochondrial oxidation, demonstrating that the mature cells were dependent on FAO. However, the fact that we observed no difference in the OCR in the presence of palmitate acid as compared to bovine serum albumin (BSA) suggests that these cells were not using exogenous fatty acids, but rather, relied on an endogenous source (FIGS. 3A and 3B). Similar patterns were observed in the DT-treated cells, indicating that the hormonal stimuli promote the use of endogenous FA rather than exogenous FA. In contrast to the controls, the mature cells generated herein showed no spare energy capacity, indicating that at a basal level, they were oxidizing substrates at maximal capacity (FIG. 3B). Additionally, these cells also had a significant amount of proton leak, known to be activated by increased rates of oxidative phosphorylation (Brand et al., Biochem J. (2005) 392:353-62).

In humans, the energy source of the heart changes from lactate supplied by the placenta to lipid-rich milk following birth. This change in energy source results in dramatic changes in the circulating fatty acid concentration. Additionally, there are changes in the pO₂ of the newborn caused by the closure of various hemodynamic shunts and fetal respiration, as the pulmonary respiration system shifts from mother to newborn. These changes are accompanied by an increase in the expression of Uncoupling Proteins (UCP) to compensate for the increased oxidative stress in the newborn. RT-qPCR quantification of the two predominate UCP isoforms in the heart, UCP2 and UCP3, showed that there was a significant upregulation of their expression in the mature cells generated herein compared to the age-matched control cells (high glucose) (FIG. 3C, top two panels). Further RT-qPCR analyses showed that the PPDT-treated cells expressed higher levels of UCP2 than the other populations and higher levels of SOD2 than the control and Pal-treated cells (FIG. 3C, bottom panels).

The apparent use of endogenous FA as an energy source may be reflective of the pattern of FA usage in the neonatal heart, as the cells are transitioning from glycolysis to FAO. Immediately after birth, lipid droplets accumulate within the cardiomyocytes, likely serving as an endogenous lipid reserve as the cells undergo the switch to FAO. Transmission electron microscopy (TEM) analyses revealed that the hPSC-derived mature cells generated herein, also contained structures resembling lipid droplets (FIG. 3D). Quantification of lipid droplet membrane area based on Nile Red staining showed that the mature cells contained a significantly larger stained area than the control cells (FIG. 3E).

The presence of lipid droplets in these cells indicate that the cells have access to internal stores of FA and as such would provide an explanation as to why we did not observed differences in the OCR in the presence of palmitate or BSA. Collectively, these findings indicate that the hPSC-derived mature compact CMs undergo changes associated with the switch to FAO observed in the newborn heart, including the metabolism of long chain fatty acids, the storage of lipids and the upregulation of anti-oxidative stress genes to protect against the effects of oxidative stress.

### Example 8: Transient Activation of FAO Pathway Improves Metabolic Profiles of Mature Compact Cardiomyocytes

The lack of spare capacity in the PPDT-treated (mature) population suggests that the prolonged maturation stimulus may be inducing an abnormal hyperactive stressed phenotype in these cells. We hypothesized that the stimuli used to induce the mature phenotype in our cell culture system subjected the cells to stressful conditions, making them utilize energy at their maximal capacity similar to cardiomyocytes in the newborn heart during their metabolic adaptation periods. To test this hypothesis and determine if manipulation of the duration of treatment could impact metabolic function of the cells, we removed specific factors from the cell cultures at day 27 for 5 days and determined whether we could restore a spare energy capacity within the cells (FIG. 4A). Specifically, we shortened the induction time from 2 weeks to 9 days and then maintained the cells in either Pal and PPARa, Pal alone or no factors for the remaining 5 days. These conditions were designed to mimic transient activation of FAO pathway observed in the neonatal heart during the early post-natal adaptation period (Buroker et al., PPAR Res. (2008) 2008:279531; Talman et al., J Am Heart Assoc. (2018) 7:e010378). Seahorse analyses revealed that some spare capacity could be observed following removal of Dex and T3 (FIG. 4B, panel 2). Cells maintained in Pal alone for the final 5 days of culture showed significant increase in maximal respiration and spare capacity, where the spare capacity increased dramatically with the removal of GW7647, Dex, and T3 (FIG. 4B, panel 3). Additionally, we now observed a large difference in OCR between cells treated with palmitate (blue line) and those treated with BSA (green line), indicating that the cells are competent to use an exogenous source of lipids for energy. The increase in spare capacity and the ability to used exogenous lipids was lost with the removal of palmitate, suggesting that the presence of long chain FAs was essential for maintaining the FAO state of the cells (FIG. 4B, panel 4). Quantification of the OCR curve parameters showed that continual culture in the presence of palmitate allowed for a high basal metabolism and proton leak and provided an increased maximal respiration and spare capacity from exogenous lipid supply in addition to endogenous lipid storage (FIG. 4C). Quantification of the OCR curve parameters further showed that cells treated for 9 days with PPDT and then cultured in Pal alone showed significantly higher maximal respiration and spare capacity compared cells treated with the other combinations of factors (FIG. 4C). Nile Red staining revealed that the shortened induction time led to a reduction in the endogenous lipid stores in the cells compared to those subjected to continuous stimulus (days 18-32). In contrast, the levels of mitochondrial related genes, such as ATP5A1, COX7A1, and CKMT2, were similar in the two populations.

These findings demonstrate that the culture conditions identified in this study to induce a mature cardiomyocyte phenotype are sufficient to generate cells that are able to oxidize exogenous fatty acids and display a metabolic phenotype similar to that of neonatal cardiomyocytes. More specifically, these findings demonstrate that induction of hPSC-derived cardiomyocytes for 9 days with PPDT followed by 5 days of culture in Pal (PPDT/PAL) (FIG. 4D) promotes metabolic maturation of the cells yielding a population that displays a metabolic phenotype similar to that of postnatal CMs including the ability to oxidize exogenous fatty acids.

### Example 9: Molecular Profiling of Mature Compact Cardiomyocytes

To further characterize the mature compact CMs, we carried out scRNAseq analysis on the PPDT/PAL treated day 32 population and compared these profiles to those from the untreated age matched immature population. The mature and immature populations used for these analyses consisted of 92% and 85% cTNT/MLC2V⁺ cells, respectively. UMAP analyses of the combined mature and immature populations identified 5 distinct clusters, three of which expressed high levels of TNNT2 (clusters 0, 1, and 3), one enriched for extracellular matrix (ECM)-related genes (cluster 2) and one (cluster 4), that expressed endoderm-related genes. In addition to TNNT2, Cluster 3 also expressed smooth muscle-related genes (FIG. 5A, Table 1). Cluster 0 expressed higher levels of FAO and mitochondrial genes, as well as muscle related genes, than cluster 1, indicating that it contains the mature CMs (FIG. 5B, Table 1).

TABLE 1
Differentially expressed genes (top 24
genes) in each cluster among all cells

| Rank | Cluster 0 | Cluster 1 | Cluster 2 | Cluster 3 | Celuster 4 |
|---|---|---|---|---|---|
| 1 | NMRK2 | MDK | TMSB4X | ACTA2 | SERPINA1 |
| 2 | CKMT2 | NREP | S100A11 | BTG1 | TTR |
| 3 | MYH6 | MT1E | SPARC | MYLK | TM4SF4 |
| 4 | COX6A2 | FXYD6 | LGALS1 | VSNL1 | CLU |
| 5 | MASP1 | NKX2-5 | COL3A1 | MYH11 | ACTG1 |
| 6 | HRC | H3F3B | ACTG1 | NES | GC |
| 7 | CMYA5 | MT1G | COL1A2 | NR2F2 | S100A10 |
| 8 | GOT1 | MT2A | FN1 | AKAP12 | BEX1 |
| 9 | NDUFB3 | BEX4 | COL6A2 | BMP2 | TMSB4X |
| 10 | ACSL1 | PTP4A3 | TIMP1 | DES | KRT19 |
| 11 | ASAH1 | TSC22D1 | EVA1B | NR2F1 | S100A11 |
| 12 | IGFBP7 | ENO3 | VIM | RRAD | CYBA |
| 13 | MYBPC3 | BANCR | TPM4 | CXCL12 | MARCKSL1 |
| 14 | TP53INP2 | CLU | TAGLN2 | A2M | SPINT2 |
| 15 | NEBL | C12orf75 | COL1A1 | SFRP1 | DLK1 |
| 16 | NPPB | FGF18 | BGN | TAGLN | TM7SF2 |
| 17 | SDHB | CNN1 | TMSB10 | MYH6 | ZFAS1 |
| 18 | DES | SLC30A1 | MARCKSL1 | CPNE5 | TXN |
| 19 | CKB | MYL6B | C11orf96 | IGFBP5 | RBP1 |
| 20 | ANKRD9 | IFI27L2 | SH3BGRL3 | ID3 | CLIC1 |
| 21 | CEL | PLCG2 | IFITM3 | PDLIM3 | HMGN1 |
| 22 | SDHA | TGFB1I1 | MARCKS | ID2 | TMSB10 |
| 23 | ACTN2 | MAGED2 | GNG11 | AEBP1 | SH3BGRL3 |
| 24 | CD36 | MEIS2 | C7 | TGFB1I1 | SOX4 |

To elucidate the differences between cluster 0 and 1, we performed Gene Ontology (GO) analysis using genes differentially expressed between those two populations. These analyses showed that the mature cells expressed higher levels of genes associated with FAO, mitochondrial function, muscle contraction and sarcomere organization than those in cluster 1, indicating that the PPDT/PAL treatment induces broad maturation changes with the cells (FIG. 5C).

More detailed analyses of the mature cardiomyocyte population (cluster 0) revealed heterogeneity that resolved into 4 distinct clusters (Table 2). The major distinguishing features of these subpopulations was the expression of stress-related genes, ATF5 and TRIB3 in cluster B, genes indicative of proliferation (MKI67 and FOXM1) in cluster C and extracellular matrix related genes such as FN1 and COL3A1 in cluster D. These findings indicate that the mature population consists of a large subpopulation of mature, non-proliferating non-stressed cardiomyocytes (Cluster A), along with a subpopulation of proliferating cells, a small subpopulation of contaminating fibroblasts and a subpopulation of stressed cells (Table 2). GO analysis using differentially expressed genes in each cluster revealed that muscle stress fiber-related genes and cholesterol import-related genes were upregulated in cluster A, while ER stress-related genes including CHOP-C/EBP complex and CHOP-ATF4 complex were upregulated in cluster B (FIG. 5D). Further analyses identified a number of genes that were expressed at higher levels in cluster A than in the other clusters including the surface markers CD36 and LDLR, the cytokine FGF12, which plays a role in adult cardiac electrophysiology and ASB2, which regulates structural maturation and cardiac tissue integrity (Fukuda et al., Nat Commun. (2017) 8, 14495; Hennessey et al., Heart Rhythm (2013) 10:1886-94) (FIG. 5E). The expression pattern of these genes, as well as others identified in this analysis provide a molecular signature for the identification of the hPSC-derived metabolically mature cells (Table 2).

TABLE 2
Differentially expressed genes (top 24 genes) in each
cluster by clustering analyses among mature CMs

| Rank | Cluster A | Cluster B | Cluster C | Cluster D |
|---|---|---|---|---|
| 1 | MYH7 | ASNS | STMN1 | TMSB4X |
| 2 | COL11A2 | PHGDH | TYMS | SPARC |
| 3 | LINC01088 | SARS | PTTG1 | COL3A1 |
| 4 | ASB2 | RNF187 | HMGN2 | FN1 |
| 5 | FGF12 | TRIB3 | CDKN3 | LGALS1 |
| 6 | HEPH | ARG2 | ANLN | COL1A1 |
| 7 | NREP | GARS | CENPM | COL1A2 |
| 8 | ALDOC | BEX2 | SMC4 | S100A11 |
| 9 | RYR2 | SHMT2 | BIRC5 | COL6A2 |
| 10 | MTRNR2L8 | NUPR1 | RRM2 | TPM4 |
| 11 | GUCY1A1 | ATF5 | FOXM1 | MARCKSL1 |
| 12 | PYGM | EIF3E | TK1 | FLNA |
| 13 | LGALS3BP | ZFAS1 | UBE2T | C7 |
| 14 | LDLR | TCEA1 | KPNA2 | POSTN |
| 15 | FNDC5 | SESN2 | CENPF | C11orf96 |
| 16 | PHACTR1 | ATF4 | ACTA2 | MGP |
| 17 | DMPK | MTHFD2 | NUSAP1 | EVA1B |
| 18 | NAV1 | HERPUD1 | DHFR | TAGLN2 |
| 19 | PCDH7 | DDIT3 | CENPW | RGS5 |
| 20 | HSPB6 | SLC3A2 | MKI67 | BGN |
| 21 | CEL | WARS | RPL39L | TIMP1 |
| 22 | ROGDI | XBP1 | HMGB2 | MARCKS |
| 23 | CD36 | IARS | PCLAF | SH3BGRL3 |
| 24 | SRL | PSAT1 | TPX2 | COLEC11 |

To confirm the differences in LDLR at the protein level, we used flow cytometric analyses to monitor its cell surface expression and compared it to that of CD36 (FIG. 5F). Neither marker was detected at day 18 and only small subpopulations of positive cells were present in the day 32 immature population. In contrast, more than 50% of the mature population expressed both markers at day 32. CD36⁺LDLR⁺ cardiomyocytes were already detected at day 25 at which point they represented ˜35% of the maturing population.

For further gene signature analysis of mature ventricular compact cardiomyocytes, heat map was generated using a gene list derived by applying a binary enrichment search to the data set based on batch identity. The search started with considering the ratio between the number of cells positive for a transcript in one batch when compared to that of another batch, normalized to the total number of cells in the respective batches. Only transcripts with a representation of >50% in the target batch and <25% in the remainder of the data set (the other batch or batches) are considered. This ratio can then be used as an enrichment score for filtering genes and deriving the final binary enrichment list. A minimum cutoff of 2 was applied. In general, this signature derivation method sought to find gene signatures that could be described as ‘on’ in one population and ‘off’ in another rather than just looking at differential expression where genes may be included when transcript levels are simply much higher in one population. A standard differential expression methodology was applied to the data in order to derive the lists of genes and do the Gene Ontology (GO) analysis. The gene set, CD36, NMRK2, NPPB, HSPB6, MASP1, HRC, ACSL1 and SCD, arose from the binary enrichment analysis. These genes were upregulated in the mature dataset that passed the search criteria. The gene set, KLF9 and CEBPB, arose from a filtering of significantly differentially expressed genes for known transcription factors. The gene ESRRA arose from a regulatory network analysis wherein gene regulatory elements were associated with the observed switch to fatty acid metabolism; ESRRA was the top regulatory element that was identified and was shown to also differentially expressed in the mature sample.

Taken together, the above data show that expression of a combination of cell surface and intracellular proteins are significantly differentially upregulated as the ventricular cardiomyocyte undergo metabolic maturation and as such are ideal markers to monitor these changes.

### Example 10: Structural and Electrophysiological Properties of the Metabolically Mature Cardiomyocytes

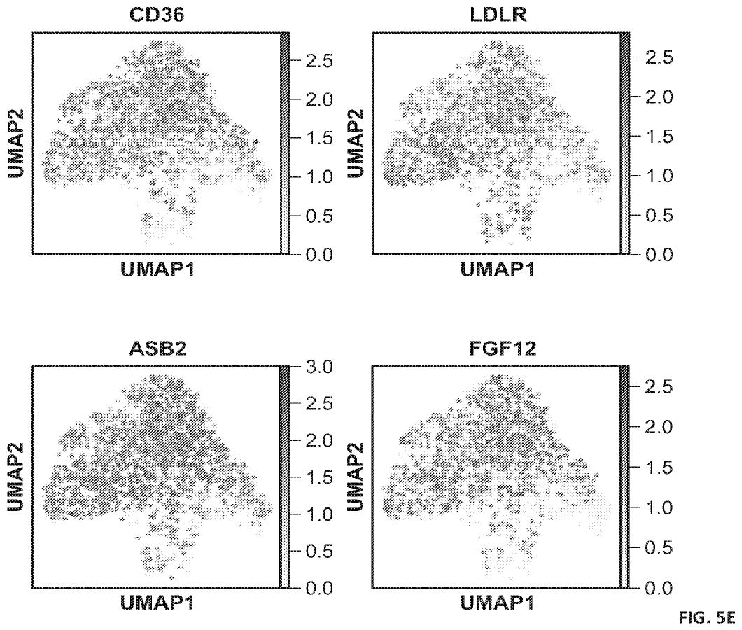

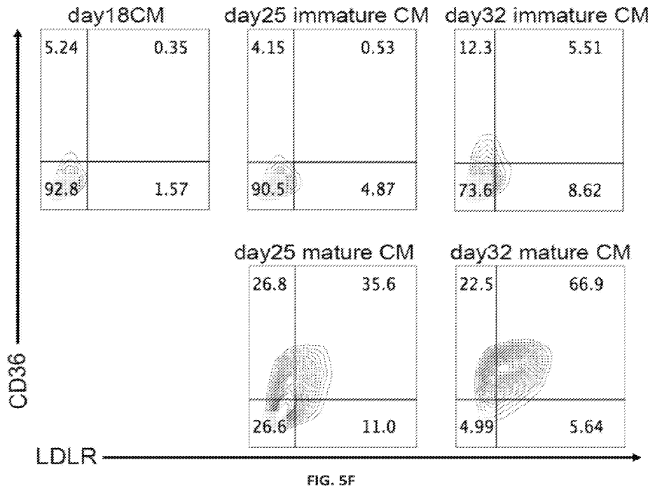

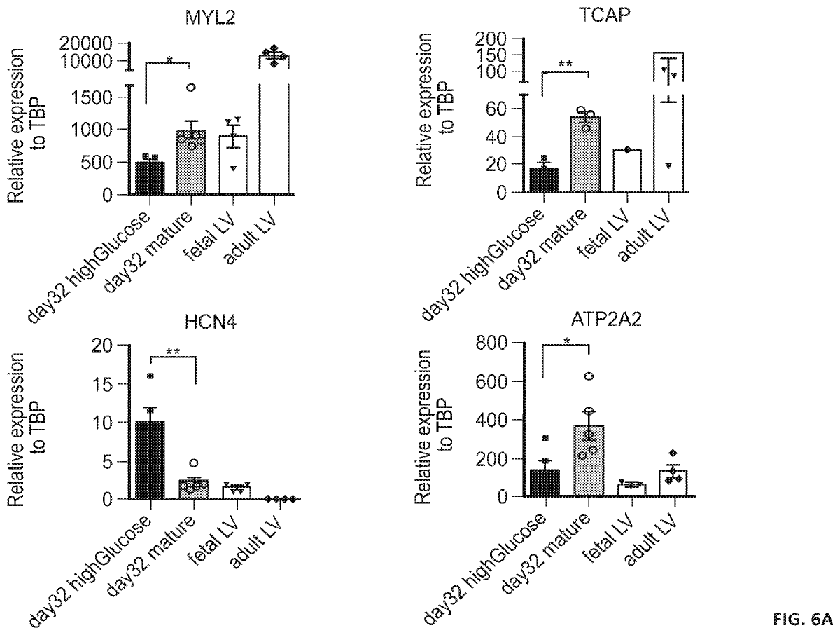

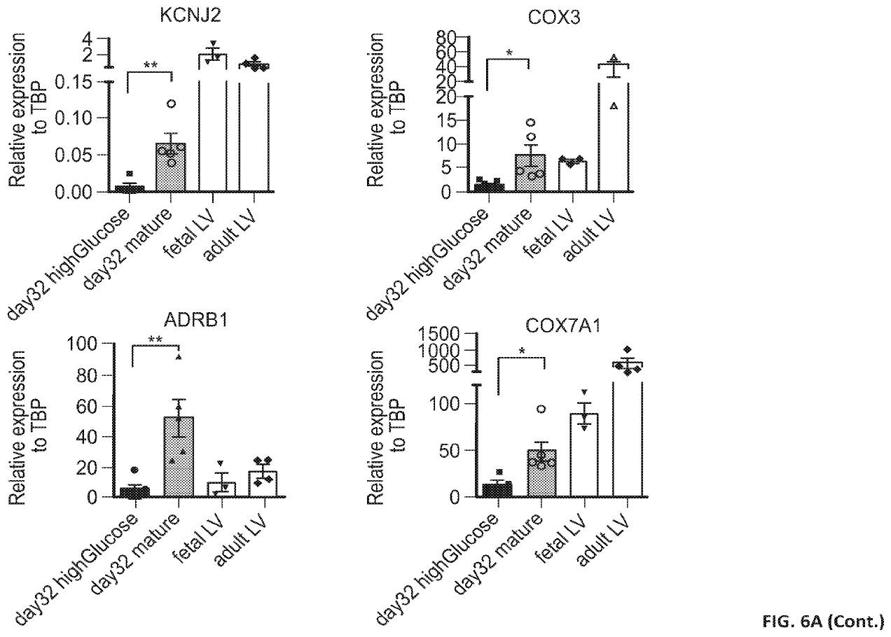

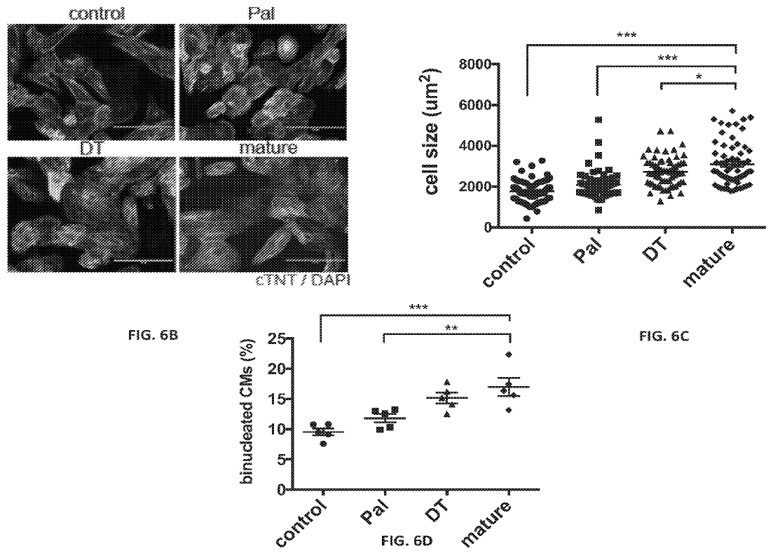

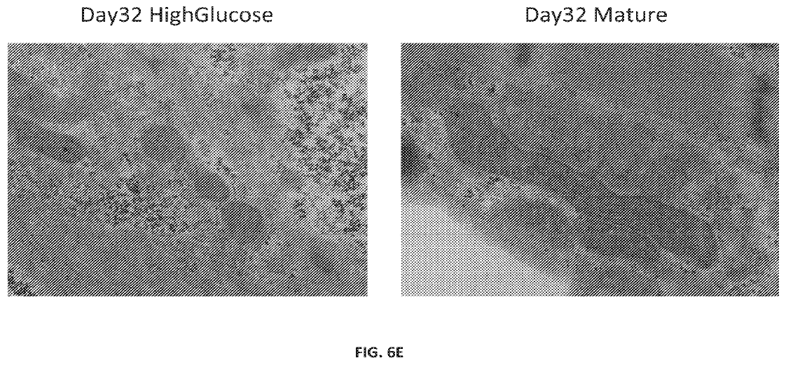

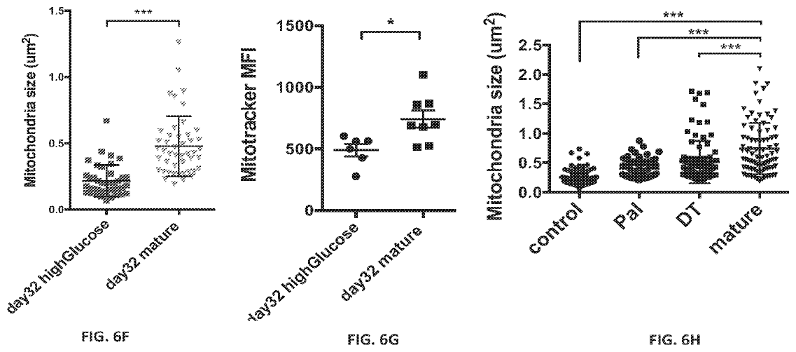

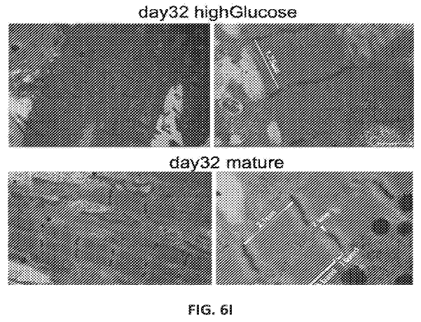

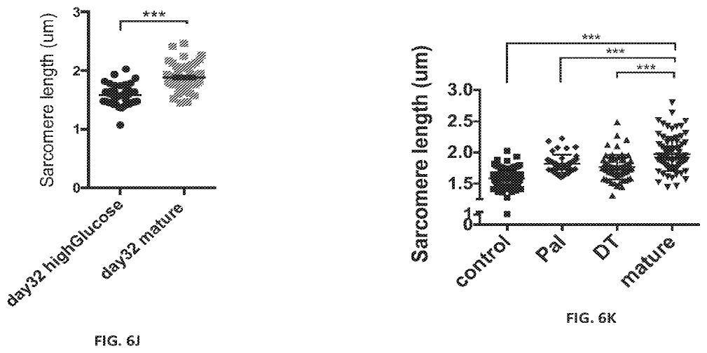

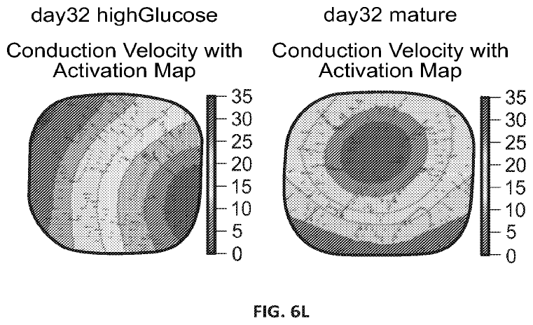

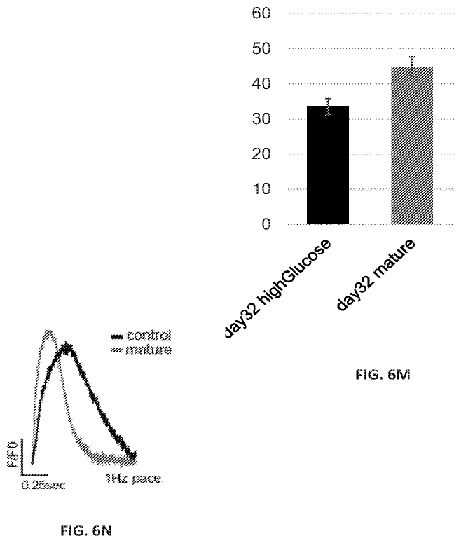

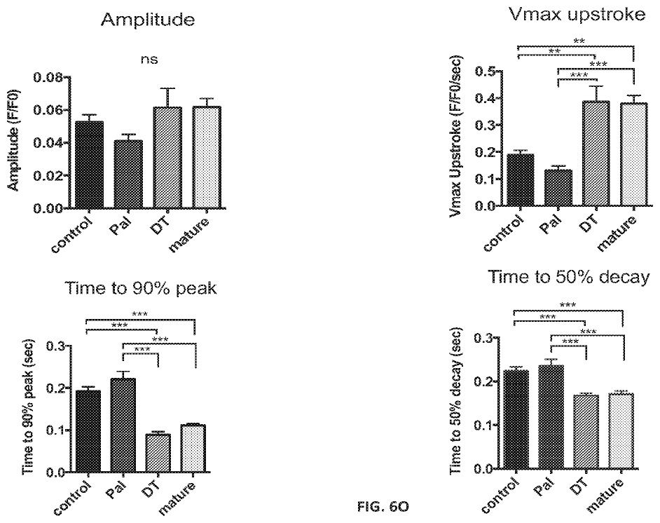

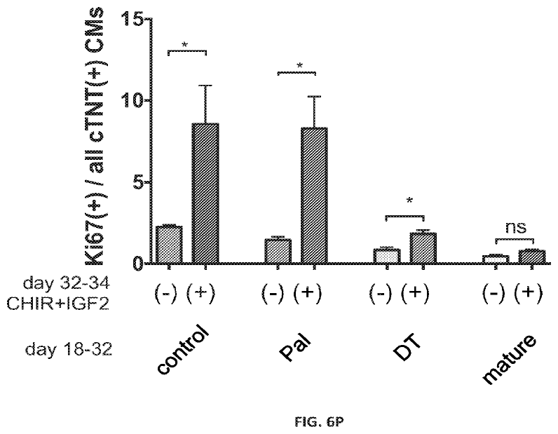

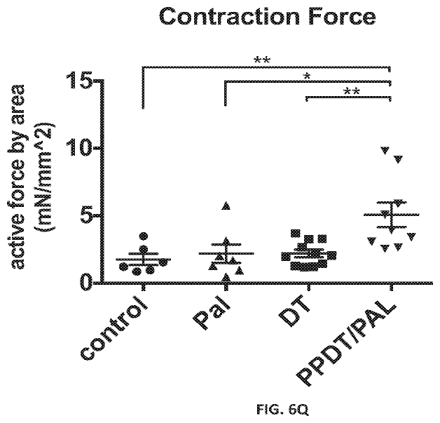

We next evaluated structural and electrophysiological properties of the metabolically mature cardiomyocytes generated herein. RT-qPCR expression analyses of cells maintained in the factors for the full 32 day culture period revealed that the mature cells expressed significantly higher expression levels of the sarcomere genes MYL2, TCAP, the ion channel gene KCNJ2, the Ca²⁺ handling gene ATP2A2, and the mitochondrial genes COX3 and COX7A1 (FIG. 6A), as compared to the control population. These mature cells upregulated the expression of ADRB1, a marker of mature cardiomyocytes (see, e.g., Tiburcy et al., Circulation (2017) 135(19):1832-47; Correia et al., Sci Rep. (2017) 7(1):8590; and Jung et al., FASEB J. (2016) 30(4):1464-79), and expressed significantly lower levels of the pacemaker current gene HCN4 than the control cardiomyocytes.

The following structural and electrophysiological analyses were carried out on cells that were switched to the palmitate-only conditions at day 27. The mature cells were larger than those in the other groups and contain more bi-nucleated cells than the control or Pal treated population (FIGS. 6B-D). TEM analyses demonstrated that these mature cardiomyocytes had larger mitochondria with more defined cristae matrix than the control “high glucose” or those treated with the other combinations of factors (FIGS. 6E and 6F). The increase in mitochondria mass was confirmed by Mitotracker staining and flow cytometry analyses (FIGS. 6G and 6H). The mature cells were larger and contained longer sarcomeres with more organized structure, including detectable Z lines, I bands, and A bands, than the control cells or those treated with the other combinations of factors (FIGS. 6B, 6C, and I-K). Electrophysiological analyses revealed that the mature cells had a higher conduction velocity as compared to the control cardiomyocytes (FIGS. 6L and 6M).

Ca²⁺ transient analyses using Fluo4 dye revealed that the mature CMs as well as those treated with DT displayed improved Ca²⁺ handling capacity compared to the untreated control and the Pal-treated CMs (FIGS. 6N and 6O). As mature cardiomyocytes are quiescent, we next measured the proportion of Ki67+ cells in each population as an additional indication of maturation status. These analyses showed that the percentage of Ki67+cTNT+CMs was significantly lower in mature and DT-treated populations than in the control or Pal-treated population. To determine if the day 32 populations contained cells that could still respond to proliferative signals, each population was treated with CHIR and IGF2 for 2 days and then analyzed them for the presence of Ki67⁺ cells (FIG. 6P). As shown in FIG. 6P, the untreated control, as well as the Pal- and DT-treated populations contain CHIR/IGF2 responsive cells. In contrast, no significant response was detected in the mature population, suggesting that these cells have lost their capacity to respond to these proliferative stimuli.

Given the observed structural differences between the cells in the various populations, we next evaluated their contraction force using engineered ‘biowire’ cardiac tissues (Nunes et al., Nat Methods (2013) 10:781-87). For these studies, biowire tissues generated with day 18 compact CMs were treated with the different combinations of factors (no factors, Pal, DT or PPDT/PAL) for two weeks and then analyzed for contraction force as previously described (Mastikhina et al., Biomaterials (2020) 233:119741). As shown in FIG. 6Q, the contraction force of the PPDT/PAL-treated tissue was significantly higher than the non-treated control tissues or those treated with Pal or DT (FIG. 6Q). TEM analyses showed that the cells in the mature tissues had more distinct, mature sarcomere structures than the cells in the tissues of the other groups. Immunohistological analyses revealed the presence of comparable proportions of cTNT+CMs and CD90+fibroblasts in the tissue constructs indicating that the differences in force are not due to dramatic differences in the proportion of these cell types.

Taken together, these results indicate that treatment with the combination of the PPARα agonist, Dex, T3, and palmitate not only facilitates the metabolic switch from glycolysis to FAO, but it also promotes structural and electrophysiological maturation in the compact cardiomyocytes. The PPDT/PAL-treated cells display more mature structural features than the untreated control cells or cells treated with Pal or DT.

### Example 11: Induction of Metabolic Maturation in HPSC-Derived Atrial Cardiomyocytes

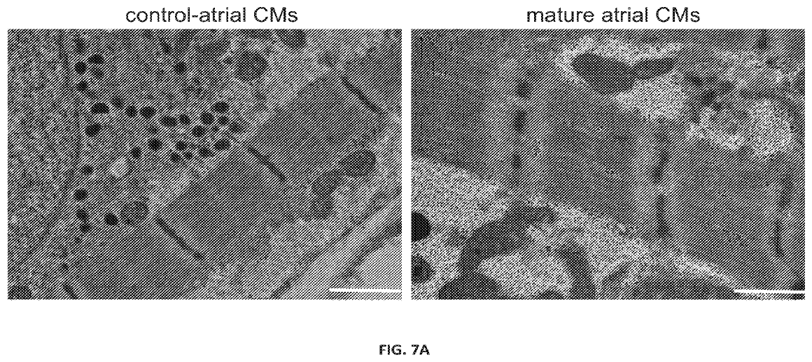

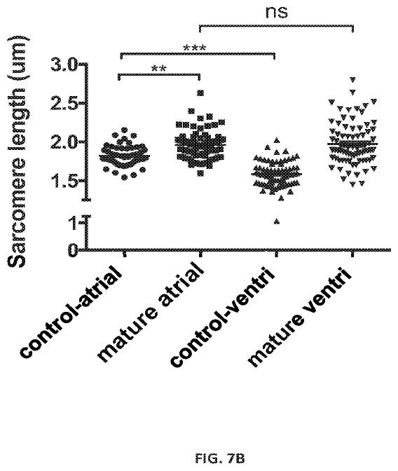

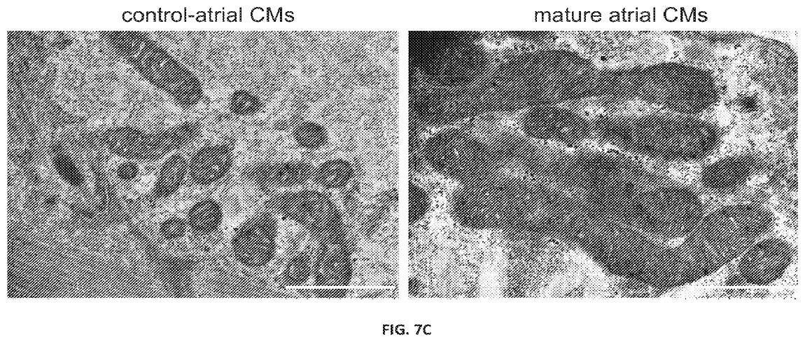

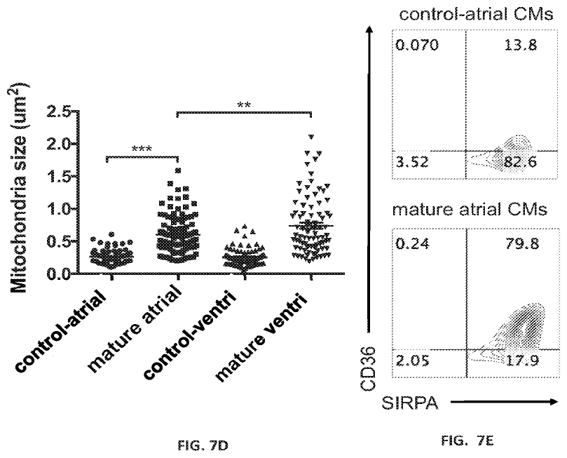

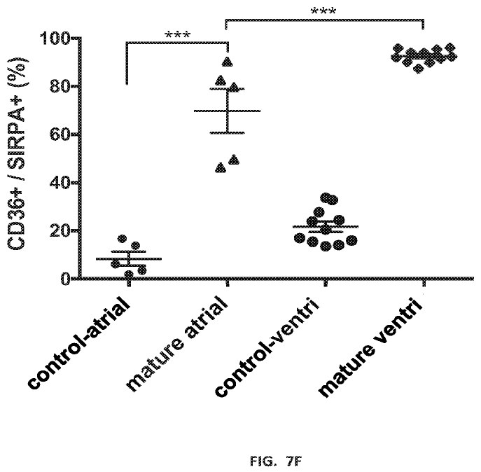

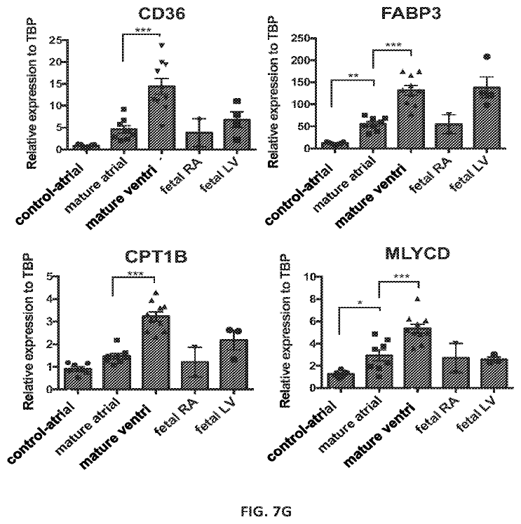

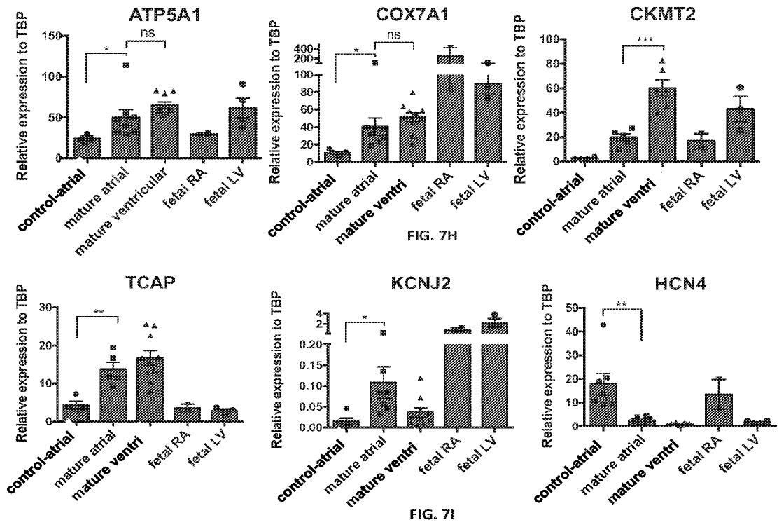

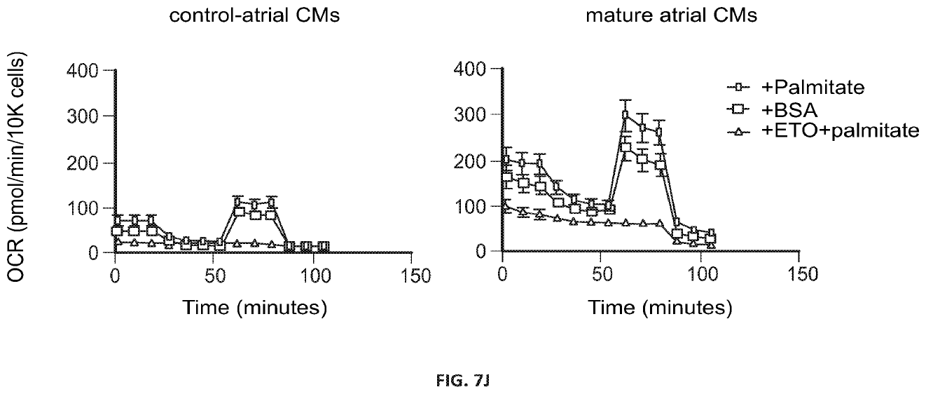

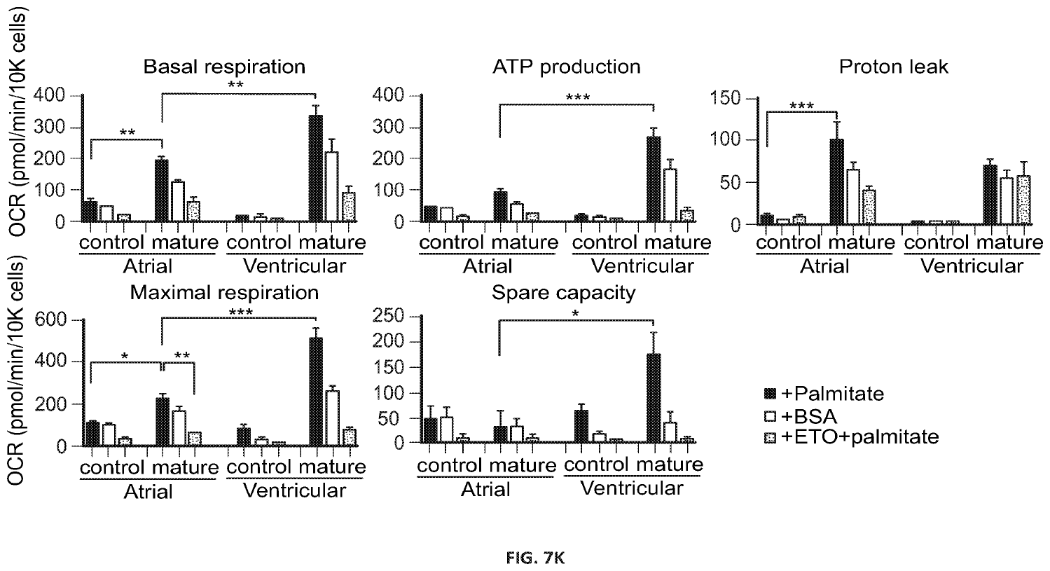

To promote maturation of other CM subtypes, we treated day 18 atrial cells generated with our previously published protocol (see, Lee supra) with the combination of PPDT/PAL for 14 days. The day 32 population expressed atrial specific genes, including KCNA5, KCNJ3, GJA5 (CX40) and NR2F2 (COUPTF2), but no MLC2V (MYL2), indicating that it consisted predominantly of atrial cells with few, if any contaminating ventricular CMs. TEM analyses showed improved sarcomere structure and increased sarcomere length in the PPDT/PAL-treated cells compared to the non-treated controls (FIGS. 7A and 7B). PPDT/PAL treatment also led to an increase in mitochondrial size in the atrial CMs, although the overall size did not reach that found in the mature ventricular cells (FIGS. 7C and 7D). As observed with the ventricular lineage cells, the PPDT/PAL-treated atrial cells also upregulated CD36, however the proportion of positive cells was somewhat lower than observed in the ventricular CM population (FIGS. 7E and 7F). Molecular analyses revealed that PPDT/PAL treatment led to an upregulation of expression of genes associated with the metabolic switch (FABP3, MLYCD) and mitochondrial activity (ATP5A1, COX7A1, CKMT2) (FIGS. 7G and 7H). The treated cells also showed higher levels of KCNJ2, an ion channel gene, and TCAP, a gene that encodes a protein that regulates sarcomere assembly than the immature cells. HCN4, the pacemaker current gene, showed an opposite pattern and was found at lower levels in the mature than in the immature cells (FIG. 7I). OCR measurements using the Seahorse™ assay showed that basal respiration, proton leak, and maximal respiration, were enhanced by the PPDT/PAL treatment of the atrial CMs. As observed with the ventricular cells, ETO (red line) blocked the mitochondrial oxidation, indicating that these atrial CMs are also dependent on FAO (FIGS. 7J and 7K). Lower Nile Red staining suggested the lower potential to store lipid in atrial CMs than in ventricular CMs. Together, these findings demonstrate that the factors that regulate maturation of the ventricular lineage cells also promote maturation of hPSC-derived atrial cells. However, the degree of change in most parameters was less than observed in the ventricular cells, which possibly reflects the fact that metabolic activity is lower in atrial CMs than in ventricular CMs in vivo.

### Example 12: Modelling Pathological Adaptation Using Mature Compact CMs

It is well established that heart failure is associated with distinct changes in myocardial metabolism, characterized by a switch from primarily FA oxidation to glycolysis (Abel and Doenst, Cardiovasc. (2011) 90:234-242; Doenst et al., Circ Res. (2013) 113:709-724). The onset of glycolysis is characterized by increased expression of GLUT1 and glyceraldehyde-3-phosphate dehydrogenase (GAPDH), resulting in the conversion of pyruvate to lactate by lactate dehydrogenase A (LDHA) (Krishnan et al., Cell Metab. (2009) 9:512-524). These metabolic changes are associated with increased concentrations of FA and lipid accumulation in the form of lipid droplets due to excessive triacylglycerol (TAG) synthesis. TAG synthesis requires free FAs and glycerol-3-phosphate generated by glycerol-3-phosphate dehydrogenase (GPD1). Glycerol-3-phosphate serves as the substrate for glycerol phosphate acyltransferase (GPAT) resulting in the TAG synthesis (Krishnan, supra). The extent of lipid accumulation is controlled in part by a family of proteins known as Perilipins (PLIN) that surround the lipid droplets and protect them from lipolysis by controlling access of lipases including the hormone sensitive lipase (HSL) that functions to release FAs from these droplets (Ueno et al., Am J Physiol Endocrinol Metab. (2017) 313, E699-E709). In advanced disease, extensive lipid accumulation within the cardiomyocytes can lead to cell death.

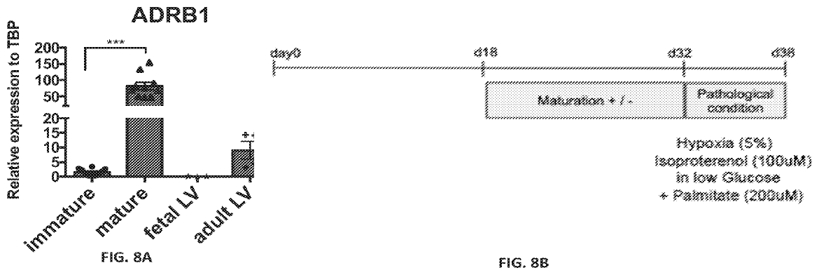

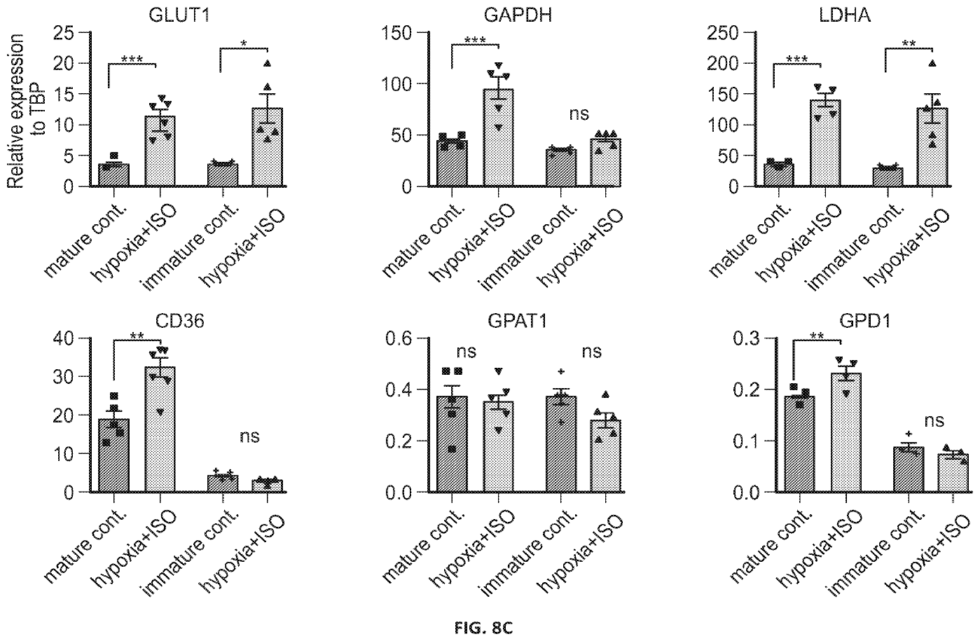

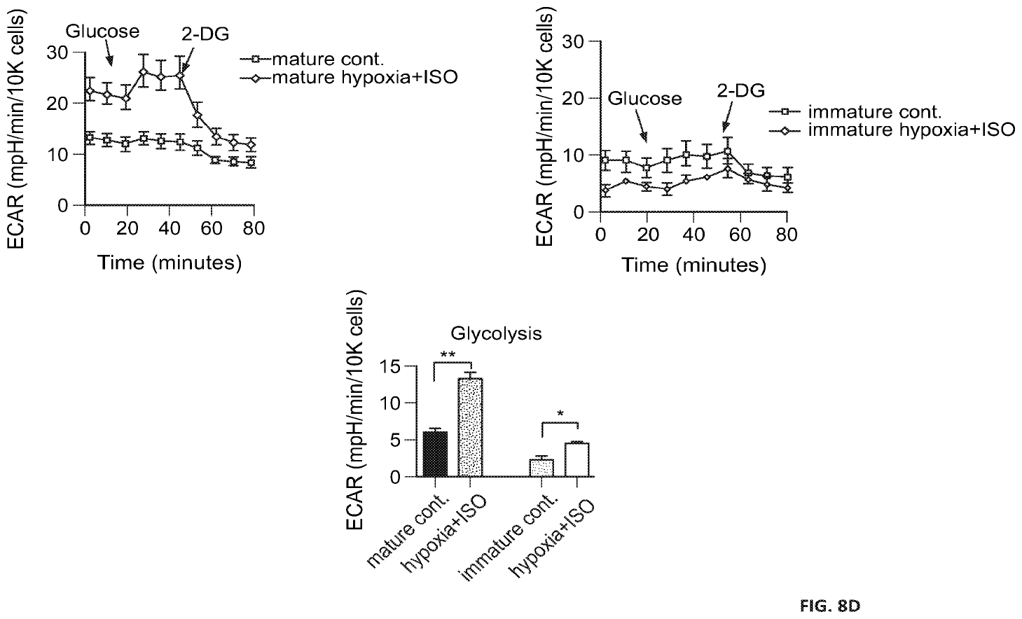

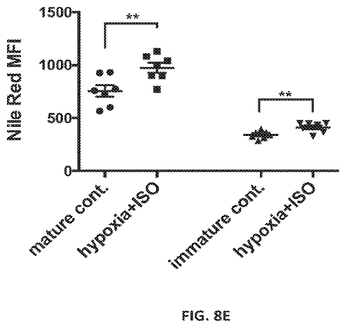

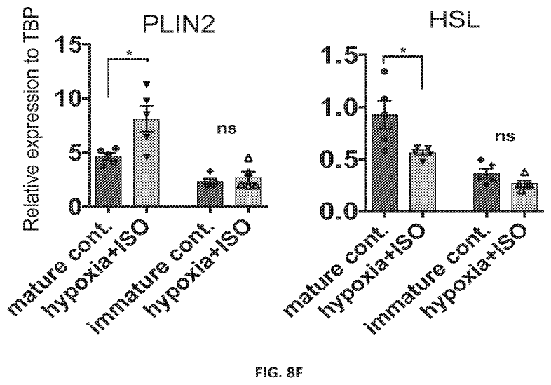

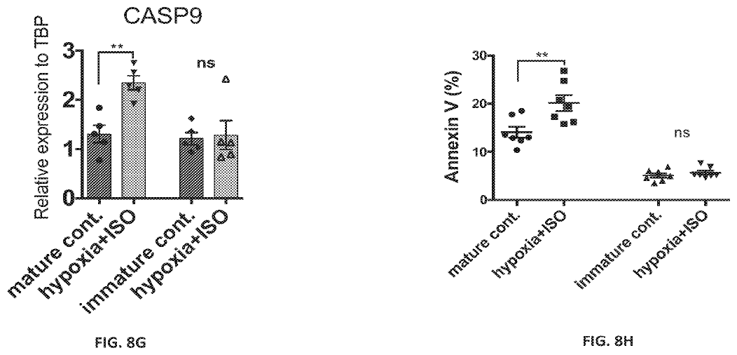

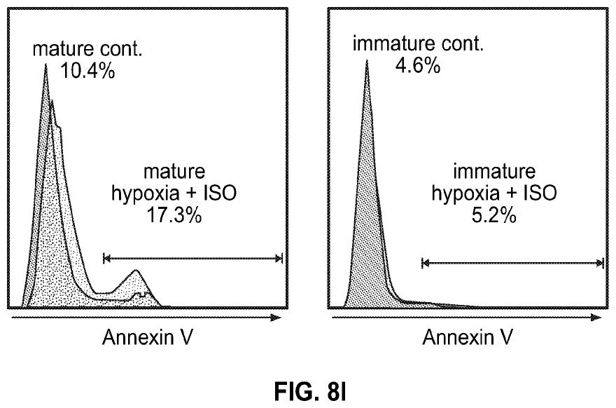

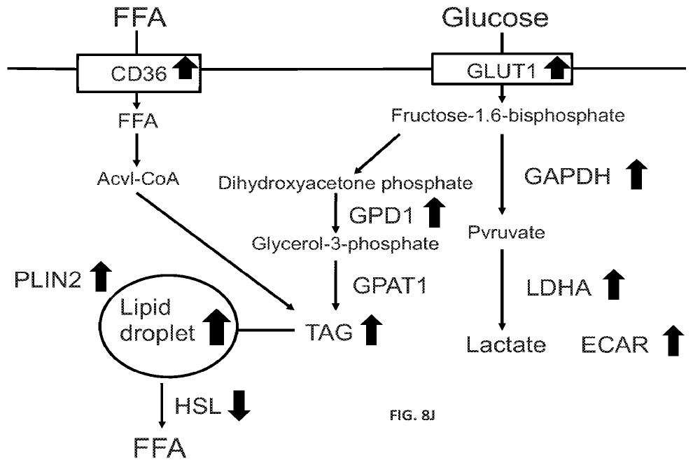

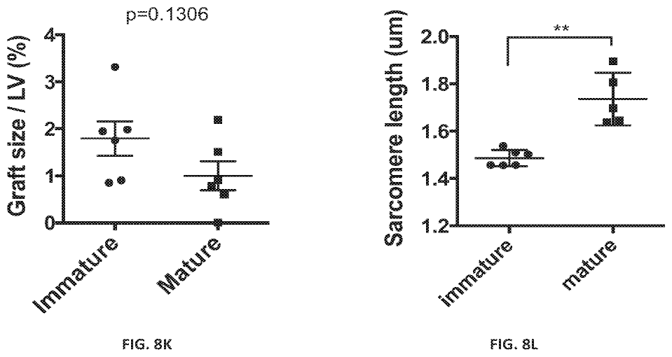

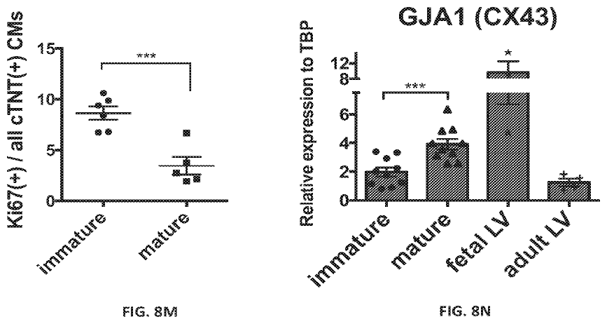

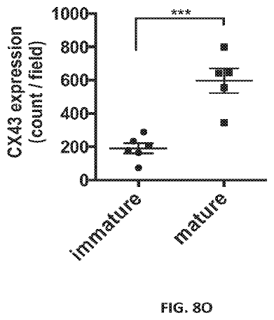

Given that the mature cardiomyocytes induced with PPDT/PAL have acquired the capacity to undergo FAO, they should provide a platform for modeling some of the above metabolic (and lipid accumulation) changes associated with heart failure. As hyperstimulation of the sympathetic adrenergic system is a characteristic heart failure and activation of the pathway is a well establish pathological stimuli, we analyzed our mature cardiomyocytes for expression of adrenergic receptor B1 (ADRB1), known to bind adrenaline and mediate responses in the adult heart. RT-qPCR analysis showed that the mature cardiomyocytes express significantly higher levels of ADRB1 than those in the immature hPSC-derived cells (FIG. 8A) indicating that they should be able to respond to appropriate stimuli.

To induce a pathological response in the mature CMs, we cultured them in the presence of isoproterenol (100 μM), a small molecule adrenergic agonist in a hypoxic (5% O2) environment, in low glucose containing medium with Palmitate (200 μM) for 6 days (FIG. 8B). Immature control cells were treated under the same conditions. This treatment induced the upregulation of expression of the glycolysis-related genes GLUT1, GAPDH, and LDHA in the mature CMs suggesting that they are undergoing a switch in metabolism (FIG. 8C). The immature cells showed similar changes in GLUT1 and LDHA expression. (FIG. 8C). To assess the glycolytic flux in these cells, we measured the extracellular acidification rate (ECAR) as an index of glycolytic activity using the Seahorse™ XF assay (Mookerjee and Brand. J Vis Exp. (2015) e53464). Mature CMs exposed to the pathological stimuli showed a rapid increase in ECAR after the injection of glucose, and a rapid decrease in ECAR following the injection of 2-deoxy-glucose (2-DG), an inhibitor of glycolysis (FIG. 8D). This response was not clearly detected in the not-treated mature CMs indicating that glycolytic activity was significantly upregulated in the mature CMs by the pathological stimuli. Quantification of glycolysis based on the ECAR measurement by the Seahorse™ assay showed these manipulations also stimulated glycolysis in the immature cells, which might be expected as those cells are dependent on glucose metabolism.

In addition to changes in metabolism, we also detected increases in lipid accumulation in the treated populations as demonstrated by Nile red (FIG. 8E). Expression of genes associated with TAG synthesis, CD36 and GPD1 as well as Perilipin 2 (PLIN2) that plays a role in abnormal lipid accumulation were only upregulated in the mature CMs cultured under the pathological stimuli (FIGS. 8E and 8F). Correspondingly, expression of the hormone sensitive lipase HSL was only downregulated in the treated mature CMs (FIG. 8F). Analyses of expression of the apoptosis-related gene CASP9 and the proportion of Annexin V⁺ cells in the populations revealed that isoproterenol/hypoxia treatment induced apoptosis in the mature but not in the immature population (FIGS. 8G-I). Taken together, these findings show that it is possible to model pathological responses in the mature CMs and that these cells recapitulate the changes associated with heart failure including activation of glycolysis, lipid accumulation and apoptosis, as summarized in FIG. 8J. The immature stimulated CMs showed a lesser degree of lipid accumulation without apoptosis and no upregulation of PLIN2 together with downregulation of HSL, which may be explained by the limited increase in glycerol-3-phosphate and the poor uptake of free FA via CD36.

### Example 13: Engraftment of Mature and Immature Cardiomyocytes into Infarcted Rat Hearts

To determine if the maturation status of the cells can influence their ability to engraft heart tissue in vivo, we transplanted both the mature and immature populations into the infarcted rat hearts. The initial strategy was to use cryopreserved cells for these studies. However, we found that the mature population was exceptionally sensitive to the cryopreservation process and as a consequence, the recovery rates were too low for transplantation. Given this, we used freshly prepared mature cells and compared them to cryopreserved immature cells. Grafts containing cTNT⁺ were detected in almost all the animals 2 weeks after transplantation except for one mature cell transplanted animal. The graft area was comparable between the two groups (FIG. 8K). Detailed analysis showed that cells in the grafts from the mature cells had significantly longer sarcomeres than those in the grafts of the immature cells (FIG. 8L). The percentage of Ki67(+) CMs was significantly lower in the mature cell grafts than in those from the immature cells (FIG. 8M), an observation consistent with the differences observed between these populations in vitro.

Given that CX43 is essential for the formation of gap junctions and electrical integration in the heart, we next analyzed our populations for CX43 message and protein. RT-qPCR analysis of the cells prior to transplantation showed higher level of CX43 message (GJA1) in the mature than the immature population (FIG. 8N). Consistent with this, immunohistiological analyses demonstrated the presence of more CX43 protein in EBs generated from the mature cells than in those from the immature control population. Analyses of the grafted cells showed that these differences persisted in vivo, with the graft from the mature population expressing more CX43 protein than the graft from the immature cells (FIG. 8O).

Although there was more CX43 protein in mature grafts, the distribution was not well organized, possibly reflecting the early stage (2 weeks) of engraftment.

Collectively, these findings indicate that the mature cell generated more mature grafts than the immature cells, suggesting that the maturation status of the transplanted cells can impact the quality of the graft.

1. A method of promoting differentiation of a ventricular cardiomyocyte progenitor cell into a ventricular compact cardiomyocyte, comprising contacting the progenitor cell with a Wnt signaling agonist and a cell proliferation stimulator, thereby obtaining a ventricular compact cardiomyocyte characterized by being HEY2⁺ANF⁻BMP10⁻.

2. The method of claim 1, wherein the cardiomyocyte progenitor cell is derived from a human pluripotent stem cell (PSC).

3. The method of claim 2, wherein the human PSC is an induced human PSC or a human embryonic stem cell.

4. The method of claim 1, wherein the Wnt signaling agonist is an inhibitor of glycogen synthase kinase-3β (GSK-3β).

5. The method of claim 4, wherein the inhibitor of GSK-3β is selected from CHIR-99021, TWS119, BIO, SB 216763, SB 415286, and CHIR-98014.

6. The method of claim 5, wherein the inhibitor of GSK-3β is CHIR-99021.

7. The method of claim 1, wherein the cell proliferation stimulator is insulin-like growth factor 2 (IGF2).

8. The method of claim 7, wherein the progenitor cell is contacted with CHIR-99021 at 0.1-10 μM and IGF2 at 1-50 ng/ml for 1-7 days.

9. The method of claim 8, wherein the progenitor cell is contacted with CHIR-99021 at about 1 μM and IGF2 at about 25 ng/ml for about six days.

10. A plurality of ventricular compact cardiomyocytes obtained by the method of claims claim 1.

11. A pharmaceutical composition consisting of a cellular component and a carrier component, wherein the cellular component is a cell population in which more than 80% of the cells are ventricular compact cardiomyocytes characterized by being HEY2⁺ANF⁻BMP10⁻, and wherein the carrier component comprises a pharmaceutically acceptably carrier,
optionally wherein the ventricular compact cardiomyocytes are characterized by being MYCN⁺.

12. A method of promoting metabolic maturation of a ventricular or atrial cardiomyocyte, comprising contacting an immature ventricular or atrial cardiomyocyte with a PPARα signaling agonist, a hydrocortisone, and a thyroid hormone, thereby obtaining a mature ventricular or atrial cardiomyocyte, respectively.

13. The method of claim 12, wherein
the immature ventricular cardiomyocyte is an immature ventricular compact cardiomyocyte, and
the mature ventricular cardiomyocyte is a mature ventricular compact cardiomyocyte characterized by being MLC2V⁺HEY2⁺ANF⁻BMP10⁻ and/or further characterized by one or both of the following features:
i) expressing, optionally at a high level, one or more markers selected from CD36, LDLR, FABP3, ACSL1, COX6A2, ATP5A1, COX7A1, CKMT2, SOD2, ASB2, FGF12, CPT1B, MLYCD, PDK4, TCAP, KCNJ2, ATP2A2, ADRB1, UCP2, UCP3, TP53INP2, NMRK2, NPPB, HSPB6, KLF9, CEBPB, MASP1, HRC, ACSL1, ESRRA, and SCD, optionally one or more markers selected from CD36, LDLR, NMRK2, NPPB, HSPB6, KLF9, CEBPB, and ESRRA; and
ii) increased (a) mitochondria mass, (b) sarcomere length, (c) conduction velocity, and/or (d) contractile force, compared to an immature ventricular compact cardiomyocyte.

14. The method of claim 12, wherein the mature atrial cardiomyocyte is characterized by being KCNA5⁺KCNJ3⁺GJA5⁺NR2F2⁺MLC2V⁻ and/or further characterized by one or both of the following features:
i) expressing, optionally at a high level, one or more of markers selected from FABP3, MLYCD, ATP5A1, COX7A1, CKMT2, KCNJ2, TCAP, and CD36; and
ii) increased (a) mitochondria mass, (b) sarcomere length, and/or (c) maximal respiration, compared to immature atrial cardiomyocytes.

15. The method of claim 12, wherein the PPARα signaling agonist is selected from GW7647, CP775146, fenofibrate, oleylethanolamide, palmitoylethanolamide, and WY14643.

16. The method of claim 15, wherein the PPARα signaling agonist is GW7647.

17. The method of claim 12, wherein the hydrocortisone is dexamethasone.

18. The method of claim 12, wherein the thyroid hormone is T3.

19. The method of claim 12, further comprising contacting the immature ventricular or atrial cardiomyocyte with a fatty acid containing 16 or more carbons.

20. The method of claim 19, wherein the fatty acid is palmitate or a derivative thereof.

21. The method of claim 12, further comprising culturing the immature ventricular or atrial cardiomyocyte in a culture medium containing glucose.

22. The method of claim 21, comprising culturing the immature ventricular or atrial cardiomyocyte in a culture medium containing GW7647, dexamethasone, thyroid hormone T3, palmitate, and glucose for a period of about one to three weeks.

23. The method of claim 22, comprising culturing the immature ventricular or atrial cardiomyocyte in a culture medium containing about 1 μM GW7647, about 100 ng/ml dexamethasone, about 4 nM thyroid hormone T3, about 200 μM palmitate, and about 2 mg/ml glucose for a period of about one to two weeks, optionally wherein the culture medium is agitated during the culturing step.

24. A method of generating a cell population enriched for mature ventricular compact cardiomyocytes, comprising
contacting a population of a ventricular cardiomyocyte progenitor cell with a Wnt signaling agonist and a cell proliferation stimulator, thereby obtaining a first cell population comprising immature ventricular compact cardiomyocytes,
contacting the first cell population with a Wnt signaling antagonist, and
culturing the contacted first cell population in the presence of a PPARα signaling agonist, a hydrocortisone, and a thyroid hormone, thereby obtaining a second cell population enriched for mature ventricular compact cardiomyocytes.

25. The method of claim 24, wherein
the immature ventricular compact cardiomyocytes are characterized by being HEY2⁺MYCN⁺ANF⁻, and/or
the mature ventricular compact cardiomyocytes are characterized by being MLC2V⁺HEY2⁺ANF⁻BMP10⁻ and optionally further characterized by one or more of the following features:
i) expressing, optionally at a high level, one or more markers selected from CD36, LDLR, FABP3, ACSL1, COX6A2, ATP5A1, COX7A1, CKMT2, SOD2, ASB2, FGF12, CPT1B, MLYCD, PDK4, TCAP, KCNJ2, ATP2A2, ADRB1, UCP2, UCP3, TP53INP2, NMRK2, NPPB, HSPB6, KLF9, CEBPB, MASP1, HRC, ACSL1, ESRRA, and SCD, optionally one or more markers selected from CD36, LDLR, NMRK2, NPPB, HSPB6, KLF9, CEBPB, and ESRRA; and
ii) increased (a) mitochondria mass, (b) sarcomere length, (c) conduction velocity, and/or (d) contractile force, compared to immature ventricular compact cardiomyocytes.

26. The method of claim 24, wherein the Wnt signaling antagonist is Xav-939.

27. The method of claim 24, comprising contacting the population of the ventricular cardiomyocyte progenitor cells with CHIR-99021 at about 1 μM and IGF2 at about 25 ng/ml for about six days to obtain the first cell population,
contacting the first cell population with about 4 μM Xav-939 for about two days, and
culturing the contacted first cell population in a culture medium containing about 1 μM GW7647, about 100 ng/ml dexamethasone, about 4 nM thyroid hormone T3, about 200 μM palmitate, and about 2 g/L glucose for a period of about two weeks, wherein the cell culture is agitated during the culturing step.

28. The method of claim 12, comprising isolating the mature cardiomyocytes from the cell culture with a first binding agent that binds LDLR and a second binding agent that binds CD36.

29. (canceled)

30. A plurality of mature cardiomyocytes obtained by the method of claim 12.

31. A pharmaceutical composition consisting of a cellular component and a carrier component, wherein the cellular component is a cell population in which more than 80% of the cells are mature ventricular compact cardiomyocytes, and wherein the carrier component comprises a pharmaceutically acceptable carrier, wherein the mature ventricular compact cardiomyocytes are characterized by being MLC2V⁺HEY2⁺ANF⁻BMP10⁻ and/or further characterized by one or both of the following features:
i) expressing, optionally at a high level, CD36, LDLR, FABP3, ACSL1, COX6A2, ATP5A1, COX7A1, CKMT2, SOD2, ASB2, FGF12, CPT1B, MLYCD, PDK4, TCAP, KCNJ2, ATP2A2, ADRB1, UCP2, UCP3, TP53INP2, NMRK2, NPPB, HSPB6, KLF9, CEBPB, MASP1, HRC, ACSL1, ESRRA, and SCD, optionally one or more markers selected from CD36, LDLR, NMRK2, NPPB, HSPB6, KLF9, CEBPB, and ESRRA; and
ii) increased (a) mitochondria mass, (b) sarcomere length, (c) conduction velocity, and/or (d) contractile force, compared to immature ventricular compact cardiomyocyte.

32. A pharmaceutical composition consisting of a cellular component and a carrier component, wherein the cellular component is a cell population in which more than 80% of the cells are mature atrial cardiomyocytes, and wherein the carrier component comprises a pharmaceutically acceptable carrier, wherein the mature atrial cardiomyocytes are characterized by mature atrial cardiomyocytes characterized by being KCNA5⁺KCNJ3⁺GJA5⁺NR2F2⁺MLC2V⁻ and/or further characterized by one or more of the following features:
i) expressing, optionally at a high level, one or more of cellular markers selected from FABP3, MLYCD, ATP5A1, COX7A1, CKMT2, KCNJ2, TCAP, and CD36; and
ii) increased (a) mitochondria mass, (b) sarcomere length, and/or (c) maximal respiration, compared to immature atrial cardiomyocytes.

33. An aggregate of cells in cell culture comprising the plurality of cells of claim 10.

34. A method of treating a cardiomyopathy condition, comprising administering to a subject in need thereof the plurality of cells of claim 10.

35. The method of claim 34, wherein the cardiomyopathy condition is myocardial infarction or heart failure, optionally wherein the heart failure is left-sided heart failure, a right-sided heart failure, a diastolic heart failure, a systolic heart failure, or congestive heart failure.

36-37. (canceled)

38. A method of detecting the presence of a mature ventricular compact cardiomyocyte in a cell population, comprising detecting a cell that expresses one or more markers selected from CD36, LDLR, FABP3, ACSL1, COX6A2, ATP5A1, COX7A1, CKMT2, SOD2, ASB2, FGF12, CPT1B, MLYCD, PDK4, TCAP, KCNJ2, ATP2A2, ADRB1, UCP2, UCP3, TP53INP2, NMRK2, NPPB, HSPB6, KLF9, CEBPB, MASP1, HRC, ACSL1, ESRRA, and SCD, wherein the detected cell is a mature ventricular compact cardiomyocyte.

39. The method of claim 38, wherein the one or more markers are selected from CD36, LDLR, NMRK2, NPPB, HSPB6, KLF9, CEBPB, and ESRRA.

40. The method of claim 39, the selected markers are (i) CD36 and LDLR; (ii) CD36 and NMRK2; or (iii) CD36, LDLR, and NMRK2.

41. The method of claim 24, comprising isolating the mature cardiomyocytes from the cell culture with a first binding agent that binds LDLR and a second binding agent that binds CD36.

42. A plurality of mature cardiomyocytes obtained by the method of claim 24.

43. An aggregate of cells in cell culture comprising the plurality of cells of claim 30.

44. An aggregate of cells in cell culture comprising the plurality of cells of claim 42.

45. A method of treating a cardiomyopathy condition, comprising administering to a subject in need thereof the plurality of cells of claim 30.

46. The method of claim 45, wherein the cardiomyopathy condition is myocardial infarction or heart failure, optionally wherein the heart failure is left-sided heart failure, a right-sided heart failure, a diastolic heart failure, a systolic heart failure, or congestive heart failure.

47. A method of treating a cardiomyopathy condition, comprising administering to a subject in need thereof the pharmaceutical composition of claim 11.

48. The method of claim 47, wherein the cardiomyopathy condition is myocardial infarction or heart failure, optionally wherein the heart failure is left-sided heart failure, a right-sided heart failure, a diastolic heart failure, a systolic heart failure, or congestive heart failure.

49. A method of treating a cardiomyopathy condition, comprising administering to a subject in need thereof the pharmaceutical composition of claim 31.

50. The method of claim 49, wherein the cardiomyopathy condition is myocardial infarction or heart failure, optionally wherein the heart failure is left-sided heart failure, a right-sided heart failure, a diastolic heart failure, a systolic heart failure, or congestive heart failure.

51. A method of treating a cardiomyopathy condition, comprising administering to a subject in need thereof the pharmaceutical composition of claim 32.

52. The method of claim 51, wherein the cardiomyopathy condition is myocardial infarction or heart failure, optionally wherein the heart failure is left-sided heart failure, a right-sided heart failure, a diastolic heart failure, a systolic heart failure, or congestive heart failure.
